(19) United States
(12) Patent Application Publication
(10) Pub. No.: US 2024/0208963 A1
(43) Pub. Date: Jun. 27, 2024

(54) CRYSTALLINE MELANOCORTIN SUBTYPE-2 RECEPTOR (MC2R) ANTAGONIST

(71) Applicant: Crinetics Pharmaceuticals, Inc., San Diego, CA (US)

(72) Inventors: Yuxin ZHAO, San Diego, CA (US); Jayachandra P. REDDY, San Diego, CA (US); Colin M. KELLY, Halifax, NS (CA)

(21) Appl. No.: 18/541,289

(22) Filed: Dec. 15, 2023

Related U.S. Application Data
(60) Provisional application No. 63/387,884, filed on Dec. 16, 2022.

(51) Int. Cl. C07D 453/02; C07C 57/145

(52) U.S. Cl. CPC C07D 453/02; C07C 57/145; C07B 2200/13

(57) ABSTRACT

Described herein are crystalline forms of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide and methods of making the same. Such forms of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide are useful in the preparation of pharmaceutical compositions for the treatment of diseases or conditions that would benefit by administration with a melanocortin subtype-2 receptor (MC2R) antagonist compound.

### CROSS REFERENCE

This application claims the benefit of U.S. Provisional Patent Application No. 63/387,884 filed Dec. 16, 2022, which is incorporated herein in its entirety by reference.

### FIELD OF THE INVENTION

Described herein are crystalline forms of a melanocortin subtype-2 receptor (MC2R) antagonist compound, as well as pharmaceutical compositions thereof, methods of making, and methods of use thereof in the treatment of diseases or conditions that would benefit with treatment with an MC2R antagonist compound.

### BACKGROUND OF THE INVENTION

The melanocortin receptors form a family of G protein-coupled receptor (GPCRs) (MC1R, MC2R, MC3R, MC4R, and MC5R) that are selectively activated by different melanocortin peptides, adrenocorticotropic hormone (ACTH), and the melanocortin peptides α-, β-, and γ-melanocyte-stimulating hormone (α-MSH, β-MSH, and γ-MSH) that are all derived proteolytically from proopiomelanocortin hormone, or POMC. ACTH is a 39 amino acid peptide that is the primary regulator of adrenal glucocorticoid synthesis and secretion and only has affinity for MC2R. As the central actor in the hypothalamic-pituitary-adrenal (HPA) axis, ACTH is secreted by the pituitary in response to stressful stimuli and acts at the adrenal gland to stimulate the synthesis and secretion of cortisol. Modulation of MC2R is attractive for the treatment of conditions, diseases, or disorders that would benefit from modulating melanocortin receptor activity.

### SUMMARY OF THE INVENTION

The present disclosure relates to various solid state forms of the MC2R receptor antagonist N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide and methods of making the same. Such forms of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide are useful for modulating the activity of MC2R receptors in mammals that would benefit from such activity.

In one aspect, described herein is a maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I). In some embodiments, the maleate salt of Compound I is crystalline.

In some embodiments, disclosed herein is a crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I).

In some embodiments, the crystalline maleate salt of Compound I is crystalline Pattern D. In some embodiments, disclosed herein is a crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern D and is characterized as having:
an X-ray powder diffraction (XRPD) pattern substantially the same as shown in FIG. 1, as measured using Cu (Kα) radiation; or
an XRPD pattern with reflections at about 4.7±0.2° 2-Theta, 9.4±0.2° 2-Theta, 11.0±0.2° 2-Theta, and 14.0±0.2° 2-Theta, as measured using Cu (Kα) radiation; or
a Differential Scanning Calorimetry (DSC) thermogram substantially the same as shown in FIG. 2; or
a simultaneous Thermogravimetric Analysis (TGA)/DSC thermogram substantially the same as shown in FIG. 3; or
a DSC thermogram with an endotherm having an onset at 161.6° C. and peak at 168.4° C.; or an endotherm with onset at 158.1° C. and peak at 167.6° C.; or
a TGA pattern with a 0.48% weight loss up to 180° C.; or
unit cell parameters substantially equal to the following at 100 K:

| Crystal System | monoclinic |
|---|---|
| Space Group | C2 |
| a | 18.7203(7) Å |
| b | 10.2473(3) Å |
| c | 38.0119(14) Å |
| α | 90° |
| β | 97.109(3)° |
| γ | 90° |
| V | 7235.9(4) Å³ |
| Z | 8 |
| Calculated Density | 1.340 Mg/m3 |
| Independent reflections | 8813; |

or
a Dynamic Vapour Sorption (DVS) isotherm plot substantially the same as shown in FIG. 4; or
a reversible mass gain of 1.14 wt. % from 2 to 95% relative humidity (RH), an unchanged XRPD after DVS analysis from 2 to 95% RH, an unchanged XRPD after storage for at least one week at 40° C. and 75% RH, or a combination thereof; or a combination thereof.

In some embodiments, crystalline Pattern D of Compound I maleate is characterized as having an XRPD pattern substantially the same as shown in FIG. 1, as measured using Cu (Kα) radiation. In some embodiments, the crystalline form is characterized as having an XRPD pattern with reflections at about 4.7±0.2° 2-Theta, 9.4±0.2° 2-Theta, 11.0±0.2° 2-Theta, and 14.0±0.2° 2-Theta, as measured using Cu (Kα) radiation. In some embodiments, the crystalline form is characterized as having: a DSC thermogram substantially the same as shown in FIG. 2; or a DSC thermogram with an endotherm having an onset at 161.6° C. and peak at 168.4° C. In some embodiments, the crystalline form is characterized as having: a simultaneous TGA/DSC thermogram substantially the same as shown in FIG. 3; or a DSC thermogram with an endotherm with onset at 158.1° C. and peak at 167.6° C.; or a TGA with a 0.48% weight loss up to 180° C. In some embodiments, the crystalline form is characterized as having unit cell parameters substantially equal to the following at 100 K:

| Crystal System | monoclinic |
|---|---|
| Space Group | C2 |
| a | 18.7203(7) Å |
| b | 10.2473(3) Å |
| c | 38.0119(14) Å |
| α | 90° |
| β | 97.109(3)° |
| γ | 90° |
| V | 7235.9(4) Å³ |
| Z | 8 |
| Calculated Density | 1.340 Mg/m3 |
| Independent reflections | 8813. |

In some embodiments, crystalline Pattern D of Compound I maleate is characterized as having: an X-ray powder diffraction (XRPD) pattern substantially the same as shown in FIG. 1, as measured using Cu (Kα) radiation; and a Differential Scanning Calorimetry (DSC) thermogram substantially the same as shown in FIG. 2; or a simultaneous Thermogravimetric Analysis (TGA)/DSC thermogram substantially the same as shown in FIG. 3.

In some embodiments, crystalline Pattern D of Compound I maleate is characterized as having: an XRPD pattern with reflections at about 4.7±0.2° 2-Theta, 9.4±0.2° 2-Theta, 11.0 0.2° 2-Theta, and 14.0±0.2° 2-Theta, as measured using Cu (Kα) radiation; and a DSC thermogram with an endotherm having an onset at 161.6° C. and peak at 168.4° C.; or an endotherm with onset at 158.1° C. and peak at 167.6° C.; or a TGA pattern with a 0.48% weight loss up to 180° C.

In some embodiments, crystalline Pattern D of Compound I maleate is anhydrous.

In some embodiments, the crystalline maleate salt of Compound I is crystalline Pattern C. In some embodiments, disclosed herein is a crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern C and is characterized as having:
an X-ray powder diffraction (XRPD) pattern substantially the same as shown in FIG. 5, as measured using Cu (Kα) radiation; or
an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; or
a Differential Scanning Calorimetry (DSC) thermogram substantially the same as shown in FIG. 6; or
a simultaneous Thermogravimetric Analysis (TGA)/DSC thermogram substantially the same as shown in FIG. 7; or
a DSC thermogram with an endotherm having an onset at 144.1° C. and peak at 150.7° C.; or an endotherm with onset at 141.7° C. and peak at 152.1° C.; or
a TGA pattern with a 0.45% weight loss up to 170° C.; or
a Dynamic Vapour Sorption (DVS) isotherm plot substantially the same as shown in FIG. 8; or
a reversible mass gain of 9.18 wt. % from 2 to 95% relative humidity (RH), an XRPD with slight changes showing some conversion to the amorphous maleate salt of Compound I after DVS analysis from 2 to 95% RH, an unchanged XRPD after storage for at least one week at 40° C. and 75% RH, or a combination thereof; or a combination thereof.

In some embodiments, crystalline Pattern C of Compound I maleate is characterized as having: an X-ray powder diffraction (XRPD) pattern substantially the same as shown in FIG. 5, as measured using Cu (Kα) radiation; and a Differential Scanning Calorimetry (DSC) thermogram substantially the same as shown in FIG. 6; or a simultaneous Thermogravimetric Analysis (TGA)/DSC thermogram substantially the same as shown in FIG. 7; or a Dynamic Vapour Sorption (DVS) isotherm plot substantially the same as shown in FIG. 8.

In some embodiments, crystalline Pattern C of Compound I maleate is characterized as having: an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; or a Differential Scanning Calorimetry (DSC) thermogram substantially the same as shown in FIG. 6; and a DSC thermogram with an endotherm having an onset at 144.1° C. and peak at 150.7° C.; or an endotherm with onset at 141.7° C. and peak at 152.1° C.; or a TGA pattern with a 0.45% weight loss up to 170° C.

In some embodiments, the crystalline maleate salt of Compound I is crystalline Pattern B. In some embodiments, disclosed herein is a crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern B and is characterized as having:
an X-ray powder diffraction (XRPD) pattern substantially the same as shown in FIG. 9, as measured using Cu (Kα) radiation; or
an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; or
a Differential Scanning Calorimetry (DSC) thermogram substantially the same as shown in FIG. 10; or
a simultaneous Thermogravimetric Analysis (TGA)/DSC thermogram substantially the same as shown in FIG. 11; or
a DSC thermogram with an endotherm having an onset at 141.4° C. and peak at 150.5° C.; or an endotherm with onset at 120.2° C. and peak at 131.4° C.; or
a TGA pattern with a >4.7% weight loss up to 200° C.; or
an XRPD that converts to Pattern C after sitting at ambient conditions for one week;
or a combination thereof.

In some embodiments, crystalline Pattern B of Compound I maleate is characterized as having: an X-ray powder diffraction (XRPD) pattern substantially the same as shown in FIG. 9, as measured using Cu (Kα) radiation; and a Differential Scanning Calorimetry (DSC) thermogram substantially the same as shown in FIG. 10; or a simultaneous Thermogravimetric Analysis (TGA)/DSC thermogram substantially the same as shown in FIG. 11.

In some embodiments, crystalline Pattern B of Compound I maleate is characterized as having: an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; and a DSC thermogram with an endotherm having an onset at 141.4° C. and peak at 150.5° C.; or an endotherm with onset at 120.2° C. and peak at 131.4° C.; or a TGA pattern with a >4.7% weight loss up to 200° C.

In some embodiments, the crystalline maleate salt of Compound I is crystalline Pattern A. In some embodiments, disclosed herein is a crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern A and is characterized as having:
an X-ray powder diffraction pattern (XRPD) substantially the same as shown in FIG. 12, as measured using Cu (Kα) radiation; or
an XRPD pattern with reflections at about 4.2±0.2° 2-Theta, 8.3±0.2° 2-Theta, and 12.5±0.2° 2-Theta, as measured using Cu (Kα) radiation; or
an XRPD that converts to Pattern C after sitting at ambient conditions for about three days; or
an XRPD that converts to Pattern C after drying in a vacuum oven at 50° C., 10⁻²-10⁻¹ Torr for 20 h;
or a combination thereof.

In some embodiments, crystalline Pattern A of Compound I maleate is characterized as having an X-ray powder diffraction pattern (XRPD) substantially the same as shown in FIG. 12, as measured using Cu (Kα) radiation.

In some embodiments, crystalline Pattern A of Compound I maleate is characterized as having an XRPD pattern with reflections at about 4.2±0.2° 2-Theta, 8.3±0.2° 2-Theta, and 12.5±0.2° 2-Theta, as measured using Cu (Kα) radiation.

In some embodiments, disclosed herein is a crystalline form of Compound I maleate that is crystalline Pattern D, and optionally further comprises: crystalline Pattern C, crystalline Pattern B, crystalline Pattern A, or amorphous maleate salt of Compound I, or a combination thereof.

In another aspect, described herein is a mandelate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I). In some embodiments, the mandelate salt of Compound I is crystalline. In some embodiments, disclosed herein is a crystalline form of the mandelate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I). In some embodiments, the crystalline mandelate salt of Compound I is crystalline Pattern E. In some embodiments, disclosed herein is a crystalline form of the mandelate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline mandelate salt of Compound I is crystalline Pattern E and is characterized as having:
an X-ray powder diffraction pattern (XRPD) substantially the same as shown in FIG. 13, as measured using Cu (Kα) radiation; or
an XRPD with X-ray diffraction pattern reflections at about 5.6±0.2° 2-Theta, 10.5±0.2° 2-Theta, 14.9±0.2° 2-Theta, and 16.5±0.2° 2-Theta, as measured using Cu (Kα) radiation; or
a Differential Scanning Calorimetry (DSC) thermogram substantially the same as shown in FIG. 14; or
a simultaneous Thermogravimetric Analysis (TGA)/DSC thermogram substantially the same as shown in FIG. 15; or
a DSC thermogram with an endotherm having an onset at 141.0° C. and peak at 152.8° C.; or an endotherm with onset at 139.8° C. and peak at 154.2° C.; or
a TGA pattern with a 1.92% weight loss up to 170° C.; or
a Dynamic Vapour Sorption (DVS) isotherm plot substantially the same as shown in FIG. 16; or
a reversible mass gain of 5.68 wt. % from 2 to 95% relative humidity (RH), an unchanged XRPD after DVS analysis from 2 to 95% RH, an unchanged XRPD after storage for at least one week at 40° C. and 75% RH, or a combination thereof; or a combination thereof.

In some embodiments, crystalline Pattern E of Compound I mandelate is characterized as having: an X-ray powder diffraction pattern (XRPD) substantially the same as shown in FIG. 13, as measured using Cu (Kα) radiation; and a Differential Scanning Calorimetry (DSC) thermogram substantially the same as shown in FIG. 14; or a simultaneous Thermogravimetric Analysis (TGA)/DSC thermogram substantially the same as shown in FIG. 15; or a Dynamic Vapour Sorption (DVS) isotherm plot substantially the same as shown in FIG. 16.

In some embodiments, crystalline Pattern E of Compound I mandelate is characterized as having: an XRPD with X-ray diffraction pattern reflections at about 5.6±0.2° 2-Theta, 10.5±0.2° 2-Theta, 14.9±0.2° 2-Theta, and 16.5±0.2° 2-Theta, as measured using Cu (Kα) radiation; and a DSC thermogram with an endotherm having an onset at 141.0° C. and peak at 152.8° C.; or an endotherm with onset at 139.8° C. and peak at 154.2° C.; or a TGA pattern with a 1.92% weight loss up to 170° C.

In another aspect, described herein is a pharmaceutical composition comprising a compound or solid state form described herein. In some embodiments, described herein is a pharmaceutical composition comprising the maleate salt or solid state form described herein; and at least one pharmaceutically acceptable excipient. In some embodiments, described herein is a pharmaceutical composition comprising the mandelate salt or solid state form described herein; and at least one pharmaceutically acceptable excipient. In some embodiments, the pharmaceutical composition is in the form of a solid form pharmaceutical composition. In some embodiments, the pharmaceutical composition is in the form of a tablet, a pill, or a capsule.

In another aspect, described herein is a process for the preparation of Crystalline Pattern D of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I) maleate:

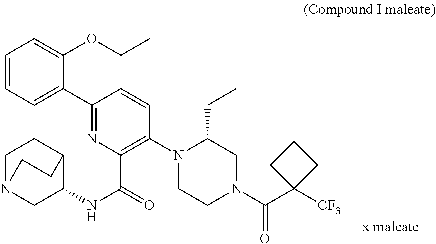 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

comprising:
   (1) contacting Compound I with maleic acid in a suitable solvent to form a mixture;
   (2) adding a suitable antisolvent to the mixture and seeding the mixture with crystals of Pattern D of Compound I maleate;
   (3) heating the mixture at a suitable temperature for a sufficient amount of time to obtain a slurry;
   (4) cooling the slurry at a suitable cooling rate; and
   (5) filtering the slurry to obtain Crystalline Pattern D of Compound I maleate.

In some embodiments, the suitable solvent in step (1) is ethanol, isopropanol, acetone, acetonitrile, methyl acetate, ethyl acetate, methyl isobutyl ketone (MIBK), water, or a combination thereof. In some embodiments, the suitable solvent of step (1) is a mixture of isopropanol and MIBK. In some embodiments, the mixture of step (1) is heated to about 50° C. In some embodiments, the mixture of step (1) comprises about 1.1 equivalents of maleic acid and about 6 volumes of a 5:1 mixture of isopropanol and MIBK, relative to the amount of Compound I in the mixture.

In some embodiments, the antisolvent in step (2) is methyl tert-butyl ether (MtBE), MIBK, water, heptane, or a combination thereof. In some embodiments, the antisolvent in step (2) is heptane. In some embodiments, from about 4 volumes to about 10 volumes of heptane relative to the amount of Compound I is added to the mixture in step (2). In some embodiments, the amount of seed crystals of Pattern D added to the mixture in step (2) is about 0.05%, about 0.10%, about 0.15%, about 0.20%, about 0.25% about 0.30%, about 0.35%, about 0.40%, about 0.45%, about 0.5%, about 0.60%, about 0.70%, about 0.80%, about 0.90%, or about 1.00%, relative to the amount of Compound I in the mixture.

In some embodiments, the mixture is heated to a temperature of from about 40° C. to about 50° C. in step (3). In some embodiments, the mixture is heated to a temperature of about 50° C. for at least 8 h, at least 12 h, at least 18 h, or more in step (3). In some embodiments, the mixture is heated to a temperature of about 50° C. for about 18 h in step (3).

In some embodiments, the slurry is cooled to a temperature of about 20° C. at a rate of at most 2.5° C./min in step (4). In some embodiments, the slurry is cooled to a temperature of about 20° C. over about 15 min, about 30 min, about 45 min, about 60 min or more in step (4). In some embodiments, the slurry is cooled to a temperature of about 20° C. over about 45 min in step (4).

In some embodiments, the Crystalline Pattern D of Compound I maleate obtained in step (5) after filtration is dried under vacuum.

In some embodiments, the method further comprises recrystallizing the Crystalline Pattern D of Compound I maleate obtained in step (5). In some embodiments, recrystallizing the Crystalline Pattern D of Compound I maleate comprises:
i. contacting Compound I Maleate Pattern D with a suitable solvent to obtain a mixture;
ii. heating the mixture of step (i) to obtain a solution;
iii. seeding the solution with crystals of Pattern D of Compound I maleate to obtain a mixture;
iv. adding a suitable antisolvent to the mixture over a suitable amount of time;
v. heating the mixture for a suitable amount of time to obtain a slurry;
vi. cooling the slurry at a suitable cooling rate; and
vii. filtering the slurry to obtain Crystalline Pattern D of Compound I maleate.

In some embodiments, the suitable solvent in step (i) is ethanol, isopropanol, acetone, methyl acetate, or a combination thereof; and wherein from about 4 volumes to about 10 volumes of solvent is used in step (i), relative to the amount of Compound I in the mixture. In some embodiments, about 4 volumes of isopropanol is used in step (i), relative to the amount of Compound I in the mixture.

In some embodiments, the mixture is heated to a temperature of from about 30° C. to about 50° C. in step (ii). In some embodiments, recrystallization further comprises cooling the solution obtained in step (ii) to a temperature of about 30° C. over about 2 hours prior to the seeding of step (iii).

In some embodiments, the amount of seed crystals of Pattern D added to the mixture in step (iii) is about 0.25%, about 0.50%, about 0.75%, about 1.0%, about 1.25%, about 1.50%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, or about 5.0%, relative to the amount of Compound I in the mixture.

In some embodiments, the suitable antisolvent of step (iv) is MtBE, heptane, or a combination thereof; and wherein from about 3 volumes to about 10 volumes of solvent is used in step (iv), relative to the amount of Compound I in the mixture. In some embodiments, the suitable antisolvent of step (iv) is MtBE; and wherein the MtBE is added over at least 1 h, at least 2 h, at least 4 h, at least 6 h, or more.

In some embodiments, in step (v) the mixture is heated to about 40° C. for about 1 h, about 2 h, or about 3 h. In some embodiments, the slurry obtained in step (v) is cooled to a temperature of about 20° C. at a rate of at most 2.5° C./min in step (vi). In some embodiments, the slurry obtained in step (v) is cooled to a temperature of about 20° C. over about 30 min, about 60 min, about 90 min, about 120 min, or more in step (vi). In some embodiments, the slurry obtained in step (v) is cooled to a temperature of about 20° C. over about 120 min in step (vi).

In some embodiments, the cooled slurry of step (vi) is maintained at about 20° C. for at least 2 h, at least, 3 h, at least 4 h, or more prior to step (vii). In some embodiments, the cooled slurry of step (vi) is maintained at about 20° C. for about 4 h prior to step (vii). In some embodiments, the Crystalline Pattern D of Compound I maleate obtained in step (vii) after filtration is dried under vacuum at a temperature of about 50° C.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 shows the X-Ray powder diffraction pattern for crystalline Pattern D of Compound I Maleate, as measured using Cu (Kα) radiation.

FIG. 2 shows the DSC thermogram for crystalline Pattern D of Compound I Maleate.

FIG. 3 shows the simultaneous TGA/DSC thermogram for crystalline Pattern D of Compound I Maleate.

FIG. 4 shows the DVS isotherm plot for crystalline Pattern D of Compound I Maleate.

FIG. 5 shows the X-Ray powder diffraction pattern for crystalline Pattern C of Compound I Maleate, as measured using Cu (Kα) radiation.

FIG. 6 shows the DSC thermogram for crystalline Pattern C of Compound I Maleate.

FIG. 7 shows the simultaneous TGA/DSC thermogram for crystalline Pattern C of Compound I Maleate.

FIG. 8 shows the DVS isotherm plot for crystalline Pattern C of Compound I Maleate.

FIG. 9 shows the X-Ray powder diffraction pattern for crystalline Pattern B of Compound I Maleate, as measured using Cu (Kα) radiation.

FIG. 10 shows the DSC thermogram for crystalline Pattern B of Compound I Maleate.

FIG. 11 shows the simultaneous TGA/DSC thermogram for crystalline Pattern B of Compound I Maleate.

FIG. 12 shows the X-Ray powder diffraction pattern for crystalline Pattern A of Compound I Maleate, as measured using Cu (Kα) radiation.

FIG. 13 shows the X-Ray powder diffraction pattern for crystalline Pattern E of Compound I Mandelate, as measured using Cu (Kα) radiation.

FIG. 14 shows the DSC thermogram for crystalline Pattern E of Compound I Mandelate.

FIG. 15 shows the simultaneous TGA/DSC thermogram for crystalline Pattern E of Compound I Mandelate.

FIG. 16 shows the DVS isotherm plot for crystalline Pattern E of Compound I Mandelate.

FIG. 17 shows a comparison of the X-Ray powder diffraction patterns for crystalline Patterns B, C, and D of Compound I Maleate, as measured using Cu (Kα) radiation.

FIG. 18 shows the X-Ray powder diffraction pattern for amorphous Compound I, as measured using Cu (Kα) radiation.

FIG. 19 shows the DSC thermogram for amorphous Compound I.

FIG. 20 shows the simultaneous TGA/DSC thermogram for amorphous Compound I.

FIG. 21 shows the thermal ellipsoid representation at the 50% probability level for all atoms in the asymmetric unit of the structure of Compound I Maleate, as determined by single crystal X-ray diffraction.

FIG. 22 shows an overlay of the predicted XRPD pattern (2) and the actual XRPD pattern from Compound I Maleate, Pattern D (1).

FIG. 23 shows the disorder of the trifluoromethyl-cyclobutyl group in the structure of Compound I Maleate, as determined by single crystal X-ray diffraction.

### DETAILED DESCRIPTION OF THE INVENTION

N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I) is a potent and selective MC2R antagonist. MC2R is a highly selective receptor for adrenocorticotropic hormone (ACTH). The major function of MC2R is to stimulate the fasciculata cells of the adrenal cortex to synthesize and secrete cortisol. MC2R antagonists are useful in the treatment of diseases or conditions for which abnormal ACTH signaling plays a role, such as Cushing's diseases, Cushing's syndrome, ectopic ACTH syndrome (EAS), and congenital adrenal hyperplasia (CAH).

### Compound I

Compound I is a potent, selective, orally available MC2R antagonist that is useful in the treatment of a variety of diseases or conditions as described herein, such as such as Cushing's diseases, Cushing's syndrome, ectopic ACTH syndrome (EAS), and congenital adrenal hyperplasia (CAH).

The preparation and uses of Compound I have been previously described (see, WO 2019/236699, U.S. Pat. Nos. 10,562,884, 10,604,507, 10,766,877, 10,981,894, U.S. Ser. No. 17/170,396, PCT/US2022/020543, and U.S. Ser. No. 17/696,279, each of which is incorporated by reference in its entirety).

Compound I refers to N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide, which has the chemical structure shown below:

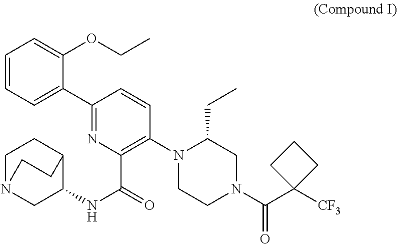 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

In some embodiments provided herein, Compound I is crystalline.

In some embodiments, disclosed herein is a salt of Compound I. In some embodiments disclosed herein, the salt of Compound I is a maleate salt or mandelate salt. In some embodiments disclosed herein, the salt of Compound I is a maleate salt. In some embodiments disclosed herein, the salt of Compound I is a mandelate salt.

In some embodiments disclosed herein, the salt of Compound I is crystalline. In some embodiments, disclosed herein is a crystalline maleate salt or crystalline mandelate salt of Compound I. In some embodiments, disclosed herein is a crystalline maleate salt of Compound I. In some embodiments, disclosed herein is a crystalline mandelate salt of Compound I.

In some embodiments provided herein, Compound I, or the salt thereof, is a single crystalline form. In some embodiments provided herein, Compound I, or the salt thereof, is a single crystalline form that is substantially free of any other crystalline form. In some embodiments, the crystalline solid form is a single solid state form, e.g. crystalline Pattern D. In some embodiments, “substantially free” means less than about 10% w/w, less than about 9% w/w, less than about 8% w/w, less than about 7% w/w, less than about 6% w/w, less than about 5% w/w, less than about 4% w/w, less than about 3% w/w, less than about 2.5% w/w, less than about 2% w/w, less than about 1.5% w/w, less than about 1% w/w, less than about 0.75% w/w, less than about 0.50% w/w, less than about 0.25% w/w, less than about 0.10% w/w, or less than about 0.05% w/w of any other crystalline form (e.g., Pattern C) in a sample of crystalline Pattern D. In some embodiments, “substantially free” means an undetectable amount (e.g., by XRPD analysis).

In some embodiments, crystallinity of a solid form is determined by X-Ray Powder Diffraction (XRPD).

### Crystalline Pattern D of Compound I Maleate Salt

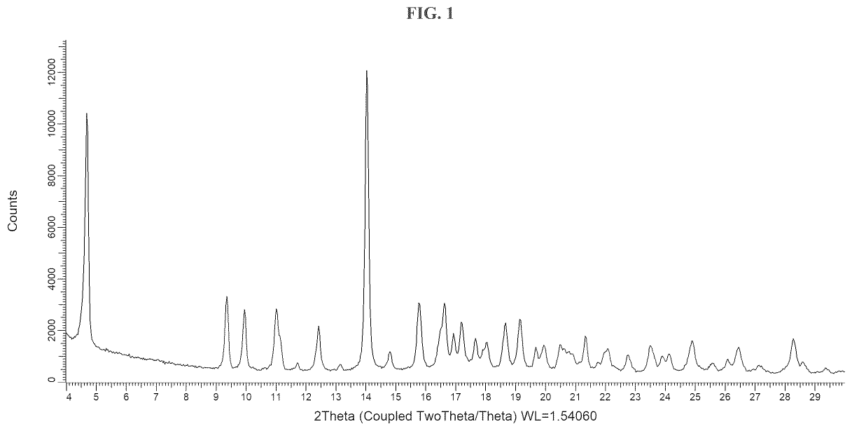

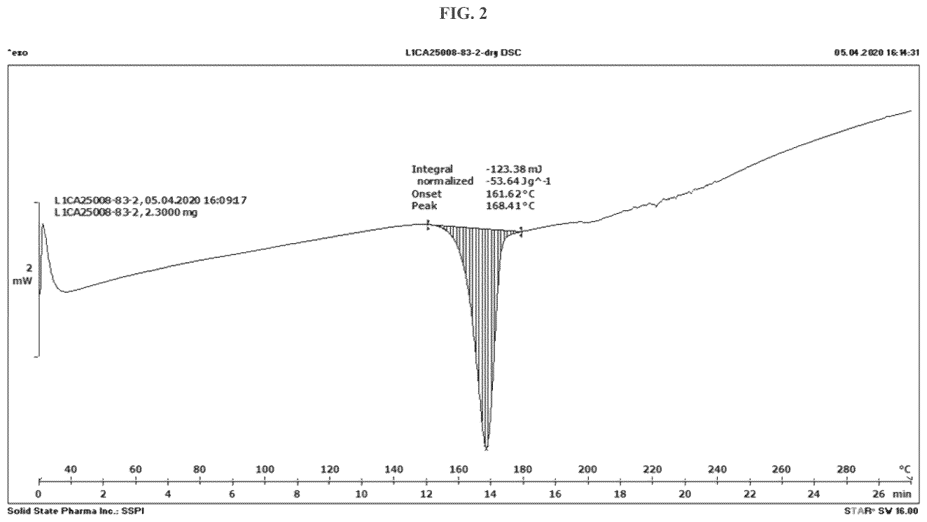

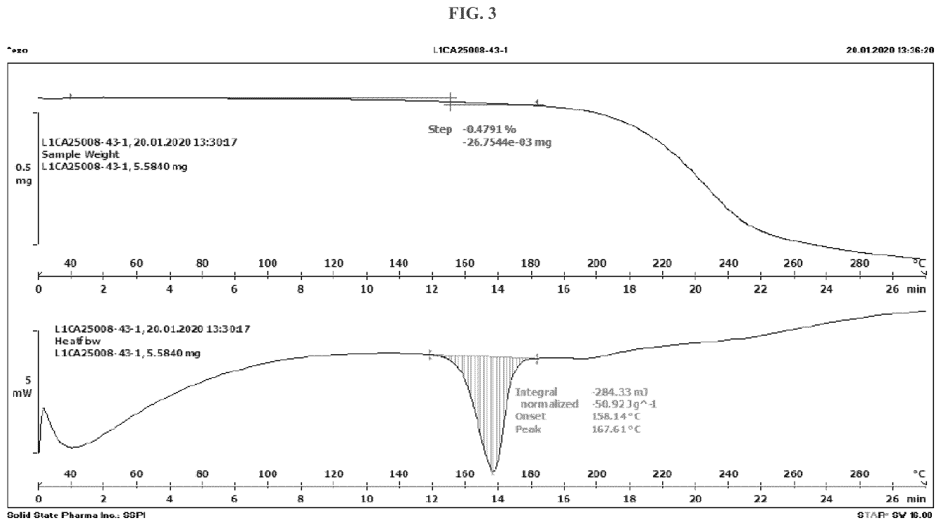

In some embodiments, provided herein is a maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), or solvate thereof, which is crystalline Pattern D. Some embodiments provide a composition comprising crystalline Pattern D of Compound I maleate. In some embodiments, crystalline Pattern D of Compound I maleate is characterized as having:
an X-ray powder diffraction (XRPD) pattern substantially the same as shown in FIG. 1, as measured using Cu (Kα) radiation; or
an XRPD pattern with reflections at about 4.7±0.2° 2-Theta, 9.4±0.2° 2-Theta, 11.0 0.2° 2-Theta, and 14.0±0.2° 2-Theta, as measured using Cu (Kα) radiation; or
a Differential Scanning Calorimetry (DSC) thermogram substantially the same as shown in FIG. 2; or
a simultaneous Thermogravimetric Analysis (TGA)/DSC thermogram substantially the same as shown in FIG. 3; or
a DSC thermogram with an endotherm having an onset at 161.6° C. and peak at 168.4° C.; or an endotherm with onset at 158.1° C. and peak at 167.6° C.; or
a TGA pattern with a 0.48% weight loss up to 180° C.; or
unit cell parameters substantially equal to the following at 100 K:

| Crystal System | monoclinic |
|---|---|
| Space Group | C2 |
| a | 18.7203(7) Å |
| b | 10.2473(3) Å |
| c | 38.0119(14) Å |
| α | 90° |
| β | 97.109(3)° |
| γ | 90° |
| V | 7235.9(4) Å³ |
| Z | 8 |
| Calculated Density | 1.340 Mg/m3 |
| Independent reflections | 8813; |

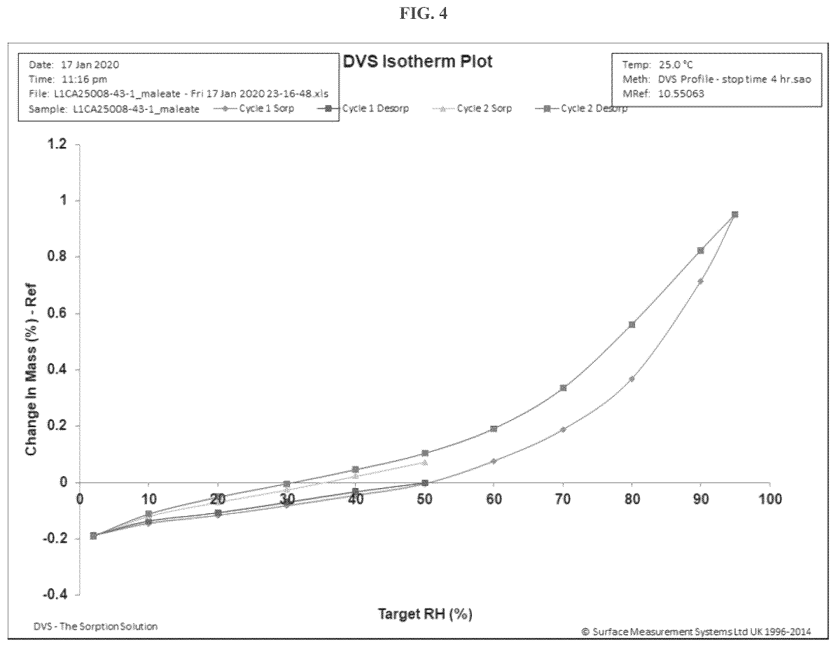

or
a Dynamic Vapour Sorption (DVS) isotherm plot substantially the same as shown in FIG. 4; or
a reversible mass gain of 1.14 wt. % from 2 to 95% relative humidity (RH), an unchanged XRPD after DVS analysis from 2 to 95% RH, an unchanged XRPD after storage for at least one week at 40° C. and 75% RH, or a combination thereof; or a combination thereof.

In some embodiments, crystalline Pattern D of Compound I maleate is characterized as having an XRPD pattern with reflections at about 4.7±0.2° 2-Theta, 9.4±0.2° 2-Theta, 11.0 0.2° 2-Theta, and 14.0±0.2° 2-Theta, as measured using Cu (Kα) radiation.

In some embodiments, crystalline Pattern D of Compound I maleate is characterized as having an XRPD pattern with reflections at about 4.7±0.2° 2-Theta, 9.4±0.2° 2-Theta, 11.0 0.2° 2-Theta, and 14.0±0.2° 2-Theta, as measured using Cu (Kα) radiation; and a DSC thermogram with an endotherm having an onset at 161.6° C. and peak at 168.4° C.; or an endotherm with onset at 158.1° C. and peak at 167.6° C.

In some embodiments, crystalline Pattern D of Compound I maleate is characterized as having an XRPD pattern with reflections at about 4.7±0.2° 2-Theta, 9.4±0.2° 2-Theta, 11.0±0.2° 2-Theta, and 14.0±0.2° 2-Theta, as measured using Cu (Kα) radiation; and a TGA pattern with a 0.48% weight loss up to 180° C.

In some embodiments, crystalline Pattern D of Compound I maleate is characterized as having an XRPD pattern with reflections at about 4.7±0.2° 2-Theta, 9.4±0.2° 2-Theta, 11.0±0.2° 2-Theta, and 14.0±0.2° 2-Theta, as measured using Cu (Kα) radiation; a DSC thermogram with an endotherm having an onset at 161.6° C. and peak at 168.4° C.; or an endotherm with onset at 158.1° C. and peak at 167.6° C.; and a TGA pattern with a 0.48% weight loss up to 180° C.

In some embodiments, crystalline Pattern D of Compound I maleate is characterized as having an XRPD pattern substantially the same as shown in FIG. 1, as measured using Cu (Kα) radiation.

In some embodiments, crystalline Pattern D of Compound I maleate is characterized as having an XRPD pattern substantially the same as shown in FIG. 1, as measured using Cu (Kα) radiation; and a Differential Scanning Calorimetry (DSC) thermogram substantially the same as shown in FIG. 2.

In some embodiments, crystalline Pattern D of Compound I maleate is characterized as having an XRPD pattern substantially the same as shown in FIG. 1, as measured using Cu (Kα) radiation; and a simultaneous TGA/DSC thermogram substantially the same as shown in FIG. 3.

In some embodiments, crystalline Pattern D of Compound I maleate is characterized as having an XRPD pattern substantially the same as shown in FIG. 1, as measured using Cu (Kα) radiation; a Differential Scanning Calorimetry (DSC) thermogram substantially the same as shown in FIG. 2, and a simultaneous TGA/DSC thermogram substantially the same as shown in FIG. 4.

In some embodiments, crystalline Pattern D of Compound I maleate is characterized as having a DSC thermogram substantially the same as shown in FIG. 2. In some embodiments, crystalline Pattern D of Compound I maleate is characterized as having a DSC thermogram with an endotherm having an onset at 161.6° C. and peak at 168.4° C. In some embodiments, crystalline Pattern D of Compound I maleate is characterized as having a simultaneous TGA/DSC thermogram substantially the same as shown in FIG. 3. In some embodiments, crystalline Pattern D of Compound I maleate is characterized as having a DSC thermogram with an endotherm with onset at 158.1° C. and peak at 167.6° C. In some embodiments, crystalline Pattern D of Compound I maleate is characterized as having a TGA pattern with a 0.48% weight loss up to 180° C. In some embodiments, crystalline Pattern D of Compound I maleate is characterized as having a DSC thermogram with an endotherm with onset at 158.1° C. and peak at 167.6° C.; and a TGA pattern with a 0.48% weight loss up to 180° C.

In some embodiments, crystalline Pattern D of Compound I maleate is characterized as having a reversible mass gain of 1.14 wt. % from 2 to 95% relative humidity (RH). In some embodiments, crystalline Pattern D of Compound I maleate is characterized as having a reversible water uptake of 1.14 wt. % from 2 to 95% relative humidity (RH). In some embodiments, crystalline Pattern D of Compound I maleate is characterized as having a reversible water uptake of about 1% from 2 to 95% relative humidity (RH).

In some embodiments, crystalline Pattern D of Compound I maleate is characterized as having an unchanged XRPD after DVS analysis from 2 to 95% RH.

In some embodiments, crystalline Pattern D of Compound I maleate is characterized as having an unchanged XRPD after storage for at least one week at 40° C. and 75% RH.

In some embodiments, crystalline Pattern D of Compound I maleate is characterized as having a reversible mass gain of 1.14 wt. % from 2 to 95% relative humidity (RH); an unchanged XRPD after DVS analysis from 2 to 95% RH; an unchanged XRPD after storage for at least one week at 40° C. and 75% RH; or a combination thereof.

In some embodiments, crystalline Pattern D of Compound I maleate is characterized as having unit cell parameters substantially equal to the following at 100 K:

| Crystal System | monoclinic |
|---|---|
| Space Group | C2 |
| a | 18.7203(7) Å |
| b | 10.2473(3) Å |
| c | 38.0119(14) Å |
| α | 90° |
| β | 97.109(3)° |
| γ | 90° |
| V | 7235.9(4) Å³ |
| Z | 8 |
| Calculated Density | 1.340 Mg/m3 |
| Independent reflections | 8813 |

In some embodiments, crystalline Pattern D of Compound I maleate is characterized as having a crystal structure characterized by atomic coordinates substantially as in Table 16; wherein the measurement of the crystal structure is carried out at 100 K. In some embodiments, crystalline Pattern D has a crystal structure characterized by unit cell parameters substantially equal to: a=18.7203(7) Å; b=10.2473(3) Å; c=38.0119(14) Å; α=90°; β=97.109(3) °; γ=90°; and having a monoclinic space group=C2; wherein the measurement of the crystal structure is carried out at 100 K. In some embodiments, crystalline Pattern D has a crystal structure characterized by unit cell parameters substantially equal to: a=18.7203(7) Å; b=10.2473(3) Å; c=38.0119(14) Å; α=90°; β=97.109(3) °; γ=90°; and having a monoclinic space group=C2; wherein the measurement of the crystal structure is carried out at 100 K and is characterized by atomic coordinates substantially as in Table 16.

In some embodiments, crystalline Pattern D of Compound I maleate is anhydrous.

In certain embodiments, described herein is a crystalline maleate salt of Compound I, or solvate thereof, that is crystalline Pattern D, and optionally further comprises: crystalline Pattern C, crystalline Pattern B, crystalline Pattern A, or amorphous maleate salt of Compound I, or solvate thereof, or a combination thereof.

### Crystalline Pattern C of Compound I Maleate Salt

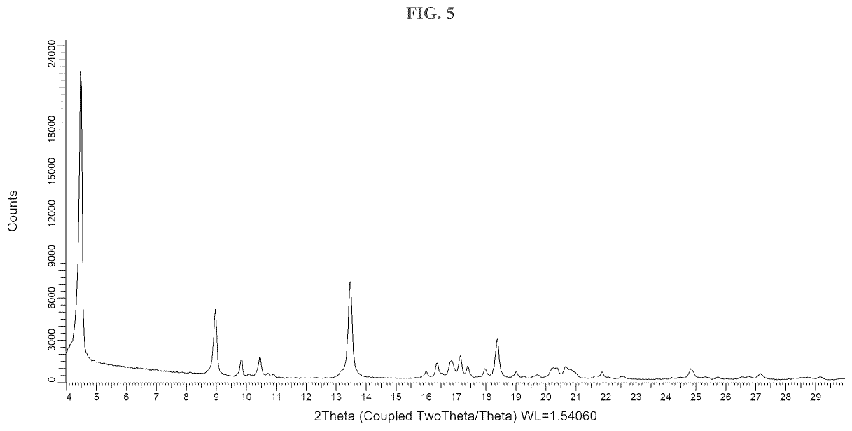

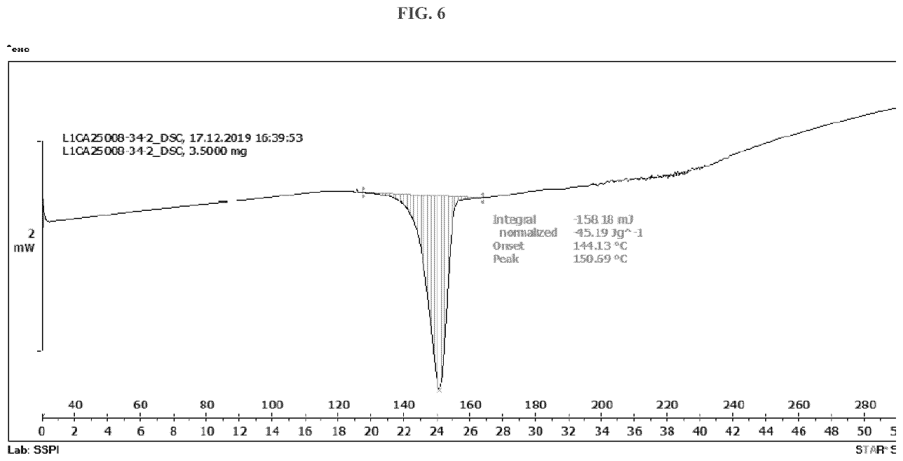

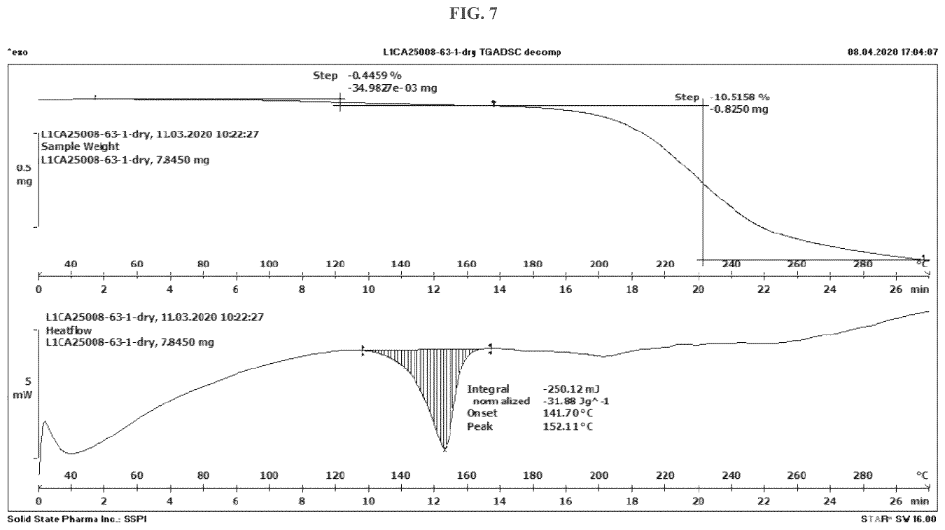

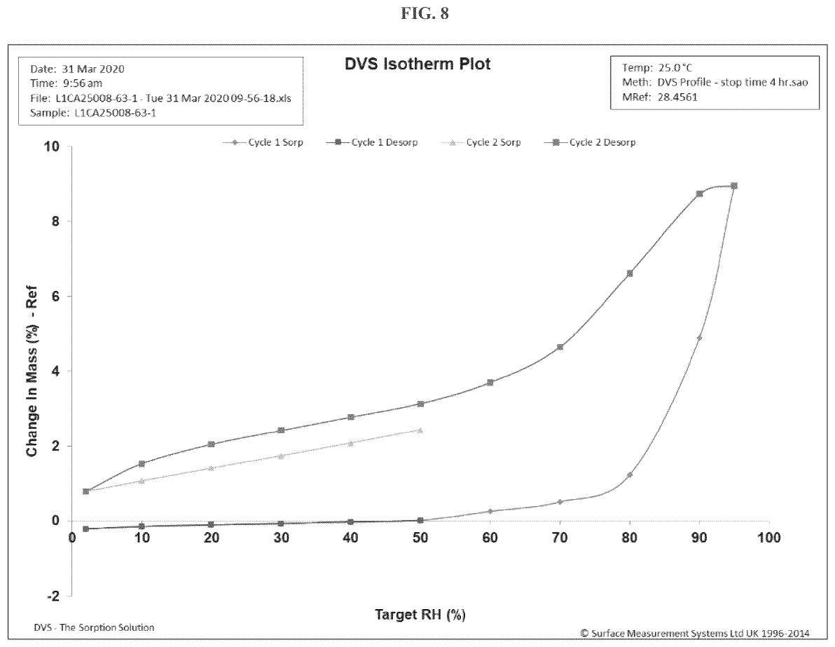

In some embodiments, provided herein is a maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), or solvate thereof, which is crystalline Pattern C. Some embodiments provide a composition comprising crystalline Pattern C of Compound I maleate. In some embodiments, crystalline Pattern C of Compound I maleate is characterized as having:
an X-ray powder diffraction (XRPD) pattern substantially the same as shown in FIG. 5, as measured using Cu (Kα) radiation; or
an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; or
a Differential Scanning Calorimetry (DSC) thermogram substantially the same as shown in FIG. 6; or
a simultaneous Thermogravimetric Analysis (TGA)/DSC thermogram substantially the same as shown in FIG. 7; or
a DSC thermogram with an endotherm having an onset at 144.1° C. and peak at 150.7° C.; or an endotherm with onset at 141.7° C. and peak at 152.1° C.; or
a TGA pattern with a 0.45% weight loss up to 170° C.; or
a Dynamic Vapour Sorption (DVS) isotherm plot substantially the same as shown in FIG. 8; or
a reversible mass gain of 9.18 wt. % from 2 to 95% relative humidity (RH), an XRPD with slight changes showing some conversion to the amorphous maleate salt of Compound I after DVS analysis from 2 to 95% RH, an unchanged XRPD after storage for at least one week at 40° C. and 75% RH, or a combination thereof;
or a combination thereof.

In some embodiments, crystalline Pattern C of Compound I maleate is characterized as having an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation.

In some embodiments, crystalline Pattern C of Compound I maleate is characterized as having an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; and a DSC thermogram with an endotherm having an onset at 144.1° C. and peak at 150.7° C.; or an endotherm with onset at 141.7° C. and peak at 152.1° C.

In some embodiments, crystalline Pattern C of Compound I maleate is characterized as having an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; and a TGA pattern with a 0.45% weight loss up to 170° C.

In some embodiments, crystalline Pattern C of Compound I maleate is characterized as having an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; a DSC thermogram with an endotherm having an onset at 144.1° C. and peak at 150.7° C.; or an endotherm with onset at 141.7° C. and peak at 152.1° C.; and a TGA pattern with a 0.45% weight loss up to 170° C.

In some embodiments, crystalline Pattern C of Compound I maleate is characterized as having an XRPD pattern substantially the same as shown in FIG. 5, as measured using Cu (Kα) radiation.

In some embodiments, crystalline Pattern C of Compound I maleate is characterized as having an XRPD pattern substantially the same as shown in FIG. 5, as measured using Cu (Kα) radiation; and a Differential Scanning Calorimetry (DSC) thermogram substantially the same as shown in FIG. 6.

In some embodiments, crystalline Pattern C of Compound I maleate is characterized as having an XRPD pattern substantially the same as shown in FIG. 5, as measured using Cu (Kα) radiation; and a simultaneous TGA/DSC thermogram substantially the same as shown in FIG. 7.

In some embodiments, crystalline Pattern C of Compound I maleate is characterized as having an XRPD pattern substantially the same as shown in FIG. 5, as measured using Cu (Kα) radiation; a Differential Scanning Calorimetry (DSC) thermogram substantially the same as shown in FIG. 6; and a simultaneous TGA/DSC thermogram substantially the same as shown in FIG. 7.

In some embodiments, crystalline Pattern C of Compound I maleate is characterized as having a DSC thermogram substantially the same as shown in FIG. 6. In some embodiments, crystalline Pattern C of Compound I maleate is characterized as having a DSC thermogram with an endotherm having an onset at 144.1° C. and peak at 150.7° C. In some embodiments, crystalline Pattern C of Compound I maleate is characterized as having a simultaneous TGA/DSC thermogram substantially the same as shown in FIG. 7. In some embodiments, crystalline Pattern C of Compound I maleate is characterized as having a DSC thermogram with an endotherm with onset at 141.7° C. and peak at 152.1° C. In some embodiments, crystalline Pattern C of Compound I maleate is characterized as having a TGA pattern with a 0.45% weight loss up to 170° C. In some embodiments, crystalline Pattern C of Compound I maleate is characterized as having a DSC thermogram with an endotherm with onset at 141.7° C. and peak at 152.1° C.; and a TGA pattern with a 0.45% weight loss up to 170° C.

In some embodiments, crystalline Pattern C of Compound I maleate is characterized as having a reversible mass gain of 9.18 wt. % from 2 to 95% relative humidity (RH). In some embodiments, crystalline Pattern C of Compound I maleate is characterized as having a reversible water uptake of 9.18 wt. % from 2 to 95% relative humidity (RH). In some embodiments, crystalline Pattern C of Compound I maleate is characterized as having a reversible water uptake of about 10% from 2 to 95% relative humidity (RH).

In some embodiments, crystalline Pattern C of Compound I maleate is characterized as having an XRPD with slight changes showing some conversion to the amorphous maleate salt of Compound I after DVS analysis from 2 to 95% RH.

In some embodiments, crystalline Pattern C of Compound I maleate is characterized as having an unchanged XRPD after storage for at least one week at 40° C. and 75% RH.

In some embodiments, crystalline Pattern C of Compound I maleate is characterized as having a reversible mass gain of 9.18 wt. % from 2 to 95% relative humidity (RH); an XRPD with slight changes showing some conversion to the amorphous maleate salt of Compound I after DVS analysis from 2 to 95% RH; an unchanged XRPD after storage for at least one week at 40° C. and 75% RH; or a combination thereof.

In some embodiments, crystalline Pattern C of Compound I maleate is anhydrous.

### Crystalline Pattern B of Compound I Maleate Salt

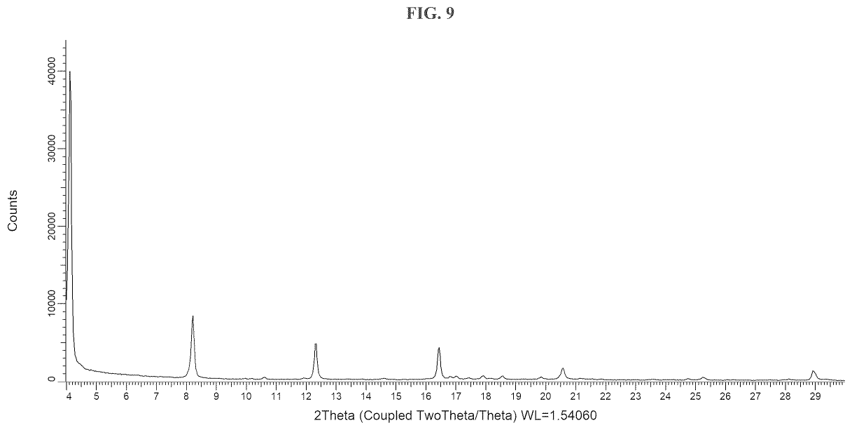

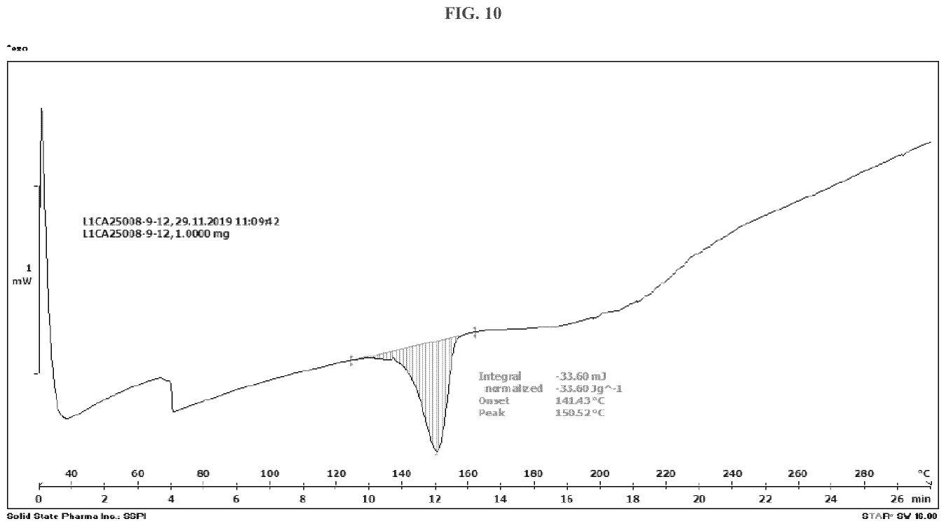

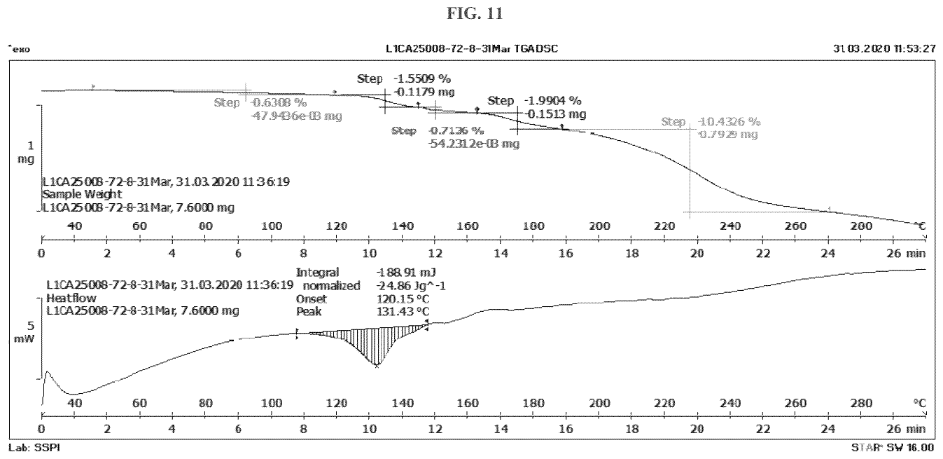

In some embodiments, provided herein is a maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), or solvate thereof, which is crystalline Pattern B. Some embodiments provide a composition comprising crystalline Pattern B of Compound I maleate. In some embodiments, crystalline Pattern B of Compound I maleate is characterized as having:
an X-ray powder diffraction (XRPD) pattern substantially the same as shown in FIG. 9, as measured using Cu (Kα) radiation; or
an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; or
a Differential Scanning Calorimetry (DSC) thermogram substantially the same as shown in FIG. 10; or
a simultaneous Thermogravimetric Analysis (TGA)/DSC thermogram substantially the same as shown in FIG. 11; or
a DSC thermogram with an endotherm having an onset at 141.4° C. and peak at 150.5° C.; or an endotherm with onset at 120.2° C. and peak at 131.4° C.; or
a TGA pattern with a >4.7% weight loss up to 200° C.; or
an XRPD that converts to Pattern C after sitting at ambient conditions for one week; or a combination thereof.

In some embodiments, crystalline Pattern B of Compound I maleate is characterized as having an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation.

In some embodiments, crystalline Pattern B of Compound I maleate is characterized as having an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; and a DSC thermogram with an endotherm having an onset at 141.4° C. and peak at 150.5° C.; or an endotherm with onset at 120.2° C. and peak at 131.4° C.

In some embodiments, crystalline Pattern B of Compound I maleate is characterized as having an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; and a TGA pattern with a >4.7% weight loss up to 200° C.

In some embodiments, crystalline Pattern B of Compound I maleate is characterized as having an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; a DSC thermogram with an endotherm having an onset at 141.4° C. and peak at 150.5° C.; or an endotherm with onset at 120.2° C. and peak at 131.4° C.; and a TGA pattern with a >4.7% weight loss up to 200° C.

In some embodiments, crystalline Pattern B of Compound I maleate is characterized as having an XRPD pattern substantially the same as shown in FIG. 9, as measured using Cu (Kα) radiation.

In some embodiments, crystalline Pattern B of Compound I maleate is characterized as having an XRPD pattern substantially the same as shown in FIG. 9, as measured using Cu (Kα) radiation; and a Differential Scanning Calorimetry (DSC) thermogram substantially the same as shown in FIG. 10.

In some embodiments, crystalline Pattern B of Compound I maleate is characterized as having an XRPD pattern substantially the same as shown in FIG. 9, as measured using Cu (Kα) radiation; and a simultaneous TGA/DSC thermogram substantially the same as shown in FIG. 11.

In some embodiments, crystalline Pattern B of Compound I maleate is characterized as having an XRPD pattern substantially the same as shown in FIG. 9, as measured using Cu (Kα) radiation; a Differential Scanning Calorimetry (DSC) thermogram substantially the same as shown in FIG. 10; and a simultaneous TGA/DSC thermogram substantially the same as shown in FIG. 11.

In some embodiments, crystalline Pattern B of Compound I maleate is characterized as having a DSC thermogram substantially the same as shown in FIG. 10. In some embodiments, crystalline Pattern B of Compound I maleate is characterized as having a DSC thermogram with an endotherm having an onset at 141.4° C. and peak at 150.5° C. In some embodiments, crystalline Pattern B of Compound I maleate is characterized as having a simultaneous TGA/DSC thermogram substantially the same as shown in FIG. 11. In some embodiments, crystalline Pattern B of Compound I maleate is characterized as having a DSC thermogram with an endotherm with onset at 120.2° C. and peak at 131.4° C. In some embodiments, crystalline Pattern B of Compound I maleate is characterized as having a TGA pattern with a >4.7% weight loss up to 200° C. In some embodiments, crystalline Pattern B of Compound I maleate is characterized as having a DSC thermogram with an endotherm with onset at 120.2° C. and peak at 131.4° C.; and a TGA pattern with a >4.7% weight loss up to 200° C.

In some embodiments, crystalline Pattern B of Compound I maleate is characterized as having an XRPD that converts to Pattern C after sitting at ambient conditions for one week.

In some embodiments, crystalline Pattern B of Compound I maleate is anhydrous.

### Crystalline Pattern A of Compound I Maleate Salt

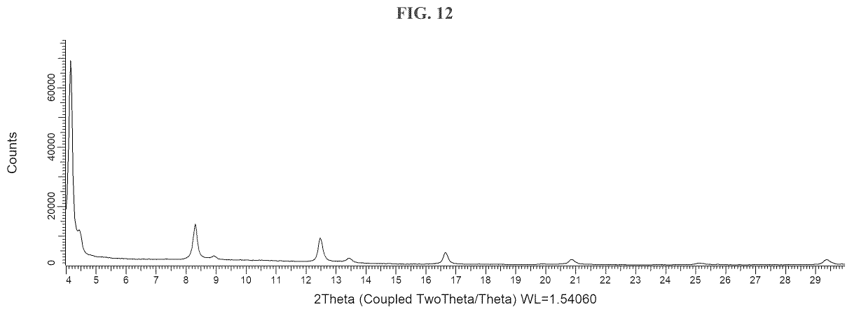

In some embodiments, provided herein is a maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), or solvate thereof, which is crystalline Pattern A. Some embodiments provide a composition comprising crystalline Pattern A of Compound I maleate. In some embodiments, crystalline Pattern A of Compound I maleate is characterized as having:
an X-ray powder diffraction pattern (XRPD) substantially the same as shown in FIG. 12, as measured using Cu (Kα) radiation; or
an XRPD pattern with reflections at about 4.2±0.2° 2-Theta, 8.3±0.2° 2-Theta, and 12.5±0.2° 2-Theta, as measured using Cu (Kα) radiation; or
an XRPD that converts to Pattern C after sitting at ambient conditions for about three days; or
an XRPD that converts to Pattern C after drying in a vacuum oven at 50° C., 10⁻²-10⁻¹ Torr for 20 h;
or a combination thereof

In some embodiments, crystalline Pattern A of Compound I maleate is characterized as having an XRPD pattern with reflections at about 4.2±0.2° 2-Theta, 8.3±0.2° 2-Theta, and 12.5±0.2° 2-Theta, as measured using Cu (Kα) radiation.

In some embodiments, crystalline Pattern A of Compound I maleate is characterized as having an XRPD pattern substantially the same as shown in FIG. 12, as measured using Cu (Kα) radiation.

In some embodiments, crystalline Pattern A of Compound I maleate is characterized as having an XRPD that converts to Pattern C after sitting at ambient conditions for about three days.

In some embodiments, crystalline Pattern A of Compound I maleate is characterized as having an XRPD that converts to Pattern C after drying in a vacuum oven at 50° C., 10⁻²-10⁻¹ Torr for 20 h.

### Crystalline Pattern E of Compound I Mandelate Salt

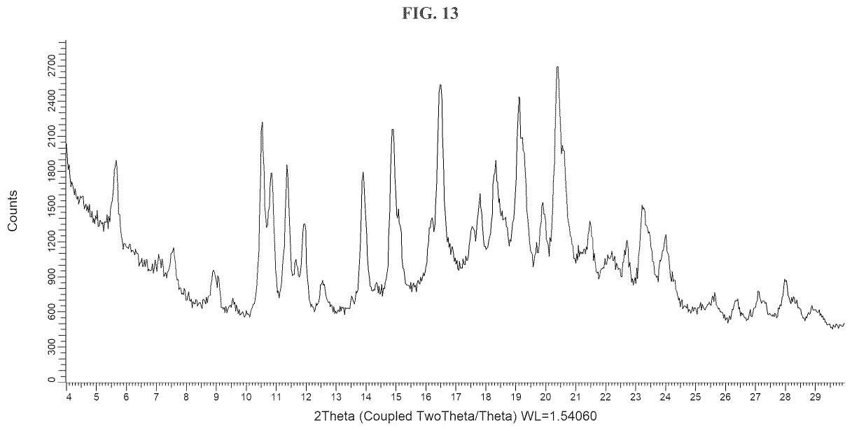

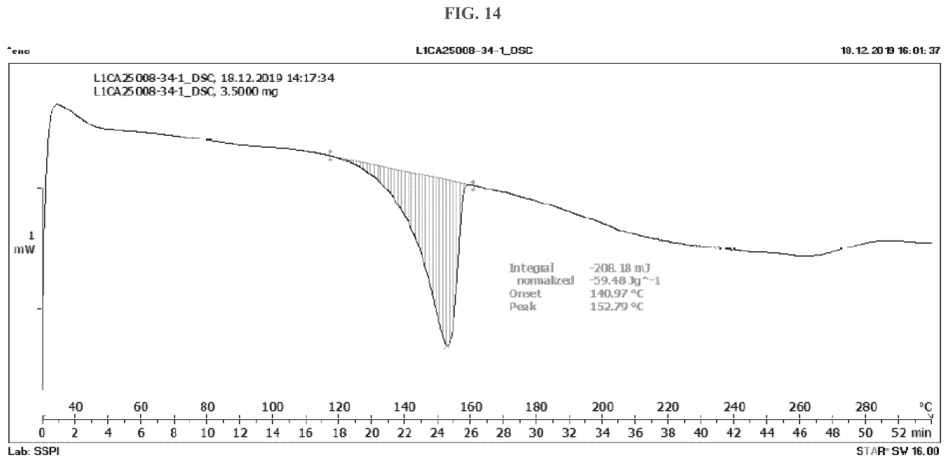

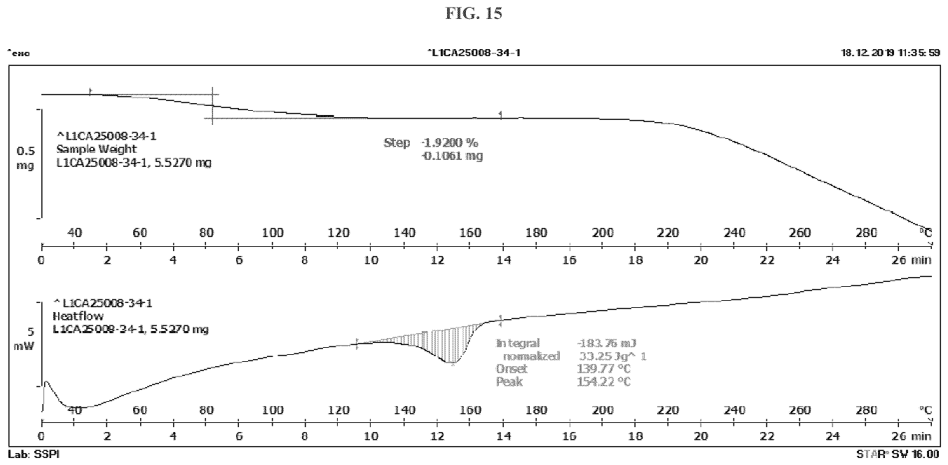

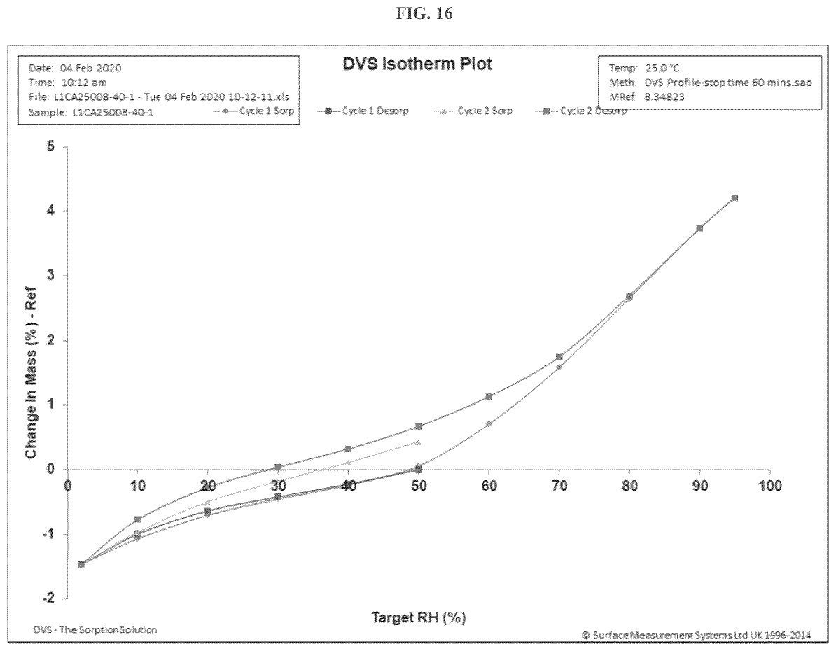

In some embodiments, provided herein is a mandelate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), or solvate thereof, which is crystalline Pattern E. Some embodiments provide a composition comprising crystalline Pattern E of Compound I mandelate. In some embodiments, crystalline Pattern E of Compound I mandelate is characterized as having:
an X-ray powder diffraction pattern (XRPD) substantially the same as shown in FIG. 13, as measured using Cu (Kα) radiation; or
an XRPD with X-ray diffraction pattern reflections at about 5.6±0.2° 2-Theta, 10.5±0.2° 2-Theta, 14.9±0.2° 2-Theta, and 16.5±0.2° 2-Theta, as measured using Cu (Kα) radiation; or
a Differential Scanning Calorimetry (DSC) thermogram substantially the same as shown in FIG. 14; or
a simultaneous Thermogravimetric Analysis (TGA)/DSC thermogram substantially the same as shown in FIG. 15; or
a DSC thermogram with an endotherm having an onset at 141.0° C. and peak at 152.8° C.; or an endotherm with onset at 139.8° C. and peak at 154.2° C.; or
a TGA pattern with a 1.92% weight loss up to 170° C.; or
a Dynamic Vapour Sorption (DVS) isotherm plot substantially the same as shown in FIG. 16; or
a reversible mass gain of 5.68 wt. % from 2 to 95% relative humidity (RH), an unchanged XRPD after DVS analysis from 2 to 95% RH, an unchanged XRPD after storage for at least one week at 40° C. and 75% RH, or a combination thereof;
or a combination thereof.

In some embodiments, crystalline Pattern E of Compound I mandelate is characterized as having an XRPD pattern with reflections at about 5.6±0.2° 2-Theta, 10.5±0.2° 2-Theta, 14.9±0.2° 2-Theta, and 16.5±0.2° 2-Theta, as measured using Cu (Kα) radiation.

In some embodiments, crystalline Pattern E of Compound I mandelate is characterized as having an XRPD pattern with reflections at about 5.6±0.2° 2-Theta, 10.5±0.2° 2-Theta, 14.9±0.2° 2-Theta, and 16.5±0.2° 2-Theta, as measured using Cu (Kα) radiation; and a DSC thermogram with an endotherm having an onset at 141.0° C. and peak at 152.8° C.; or an endotherm with onset at 139.8° C. and peak at 154.2° C.

In some embodiments, crystalline Pattern E of Compound I mandelate is characterized as having an XRPD pattern with reflections at about 5.6±0.2° 2-Theta, 10.5±0.2° 2-Theta, 14.9±0.2° 2-Theta, and 16.5±0.2° 2-Theta, as measured using Cu (Kα) radiation; and a TGA pattern with a 1.92% weight loss up to 170° C.

In some embodiments, crystalline Pattern E of Compound I mandelate is characterized as having an XRPD pattern with reflections at about 5.6±0.2° 2-Theta, 10.5±0.2° 2-Theta, 14.9±0.2° 2-Theta, and 16.5±0.2° 2-Theta, as measured using Cu (Kα) radiation; a DSC thermogram with an endotherm having an onset at 141.0° C. and peak at 152.8° C.; or an endotherm with onset at 139.8° C. and peak at 154.2° C.; and a TGA pattern with a 1.92% weight loss up to 170° C.

In some embodiments, crystalline Pattern E of Compound I mandelate is characterized as having an XRPD pattern substantially the same as shown in FIG. 13, as measured using Cu (Kα) radiation.

In some embodiments, crystalline Pattern E of Compound I mandelate is characterized as having an XRPD pattern substantially the same as shown in FIG. 13, as measured using Cu (Kα) radiation; and a Differential Scanning Calorimetry (DSC) thermogram substantially the same as shown in FIG. 14.

In some embodiments, crystalline Pattern E of Compound I mandelate is characterized as having an XRPD pattern substantially the same as shown in FIG. 13, as measured using Cu (Kα) radiation; and a simultaneous TGA/DSC thermogram substantially the same as shown in FIG. 15.

In some embodiments, crystalline Pattern E of Compound I mandelate is characterized as having an XRPD pattern substantially the same as shown in FIG. 13, as measured using Cu (Kα) radiation; a Differential Scanning Calorimetry (DSC) thermogram substantially the same as shown in FIG. 14; and a simultaneous TGA/DSC thermogram substantially the same as shown in FIG. 15.

In some embodiments, crystalline Pattern E of Compound I mandelate is characterized as having a DSC thermogram substantially the same as shown in FIG. 14. In some embodiments, crystalline Pattern E of Compound I mandelate is characterized as having a DSC thermogram with an endotherm having an onset at 141.0° C. and peak at 152.8° C. In some embodiments, crystalline Pattern E of Compound I mandelate is characterized as having a simultaneous TGA/DSC thermogram substantially the same as shown in FIG. 15. In some embodiments, crystalline Pattern E of Compound I mandelate is characterized as having a DSC thermogram with an endotherm with onset at 139.8° C. and peak at 154.2° C. In some embodiments, crystalline Pattern E of Compound I mandelate is characterized as having a TGA pattern with a 1.92% weight loss up to 170° C. In some embodiments, crystalline Pattern E of Compound I mandelate is characterized as having a DSC thermogram with an endotherm with onset at 139.8° C. and peak at 154.2° C.; and a TGA pattern with a 1.92% weight loss up to 170° C.

In some embodiments, crystalline Pattern E of Compound I mandelate is characterized as having a reversible mass gain of 5.68 wt. % from 2 to 95% relative humidity (RH). In some embodiments, crystalline Pattern E of Compound I mandelate is characterized as having a reversible water uptake of 5.68 wt. % from 2 to 95% relative humidity (RH). In some embodiments, crystalline Pattern E of Compound I mandelate is characterized as having a reversible water uptake of about 5% from 2 to 95% relative humidity (RH).

In some embodiments, crystalline Pattern E of Compound I mandelate is characterized as having an unchanged XRPD after DVS analysis from 2 to 95% RH.

In some embodiments, crystalline Pattern E of Compound I mandelate is characterized as having an unchanged XRPD after storage for at least one week at 40° C. and 75% RH.

In some embodiments, crystalline Pattern E of Compound I mandelate is characterized as having a reversible mass gain of 95.68 wt. % from 2 to 95% relative humidity (RH); an unchanged XRPD after DVS analysis from 2 to 95% RH; an unchanged XRPD after storage for at least one week at 40° C. and 75% RH; or a combination thereof.

In some embodiments, crystalline Pattern E of Compound I mandelate is anhydrous.

### Synthesis of Compound I

The preparation of Compound I has been previously described (e.g., see Example 31 of WO 2019/236699, and U.S. Pat. No. 10,562,884, which is incorporated by reference for such methods of making Compound I).

### Preparation of Crystalline Pattern D of Compound I Maleate

In some embodiments, described herein is a process for the preparation of Crystalline Pattern D of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I) maleate:

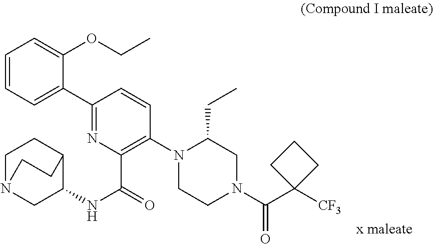 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

comprising:
   (1) contacting Compound I with maleic acid in a suitable solvent to form a mixture;
   (2) adding a suitable antisolvent to the mixture and seeding the mixture with crystals of Pattern D of Compound I maleate;
   (3) heating the mixture at a suitable temperature for a sufficient amount of time to obtain a slurry;
   (4) cooling the slurry at a suitable cooling rate; and
   (5) filtering the slurry to obtain Crystalline Pattern D of Compound I maleate.

In some embodiments, the suitable solvent in step (1) is ethanol, isopropanol, acetone, acetonitrile, methyl acetate, ethyl acetate, methyl isobutyl ketone (MIBK), water, or a combination thereof. In some embodiments, the suitable solvent of step (1) is a mixture of isopropanol and MIBK. In some embodiments, the suitable solvent of step (1) is a mixture of isopropanol and MIBK in a ratio of about 9:1, 5:1, 3:1, 2:1, 1:1, 1:2, 1:3, 1:5, or 1:9.

In some embodiments, the mixture of step (1) comprises from about 1 to about 10 volumes of solvent, relative to the amount of Compound I in the mixture. In some embodiments, the mixture of step (1) comprises from about 5 to about 10 volumes of solvent, relative to the amount of Compound I in the mixture. In some embodiments, the mixture of step (1) comprises about 5, about 6, about 7, about 8, about 9, or about 10 volumes of solvent, relative to the amount of Compound I in the mixture.

In some embodiments, the mixture of step (1) is heated to about 50° C. In other embodiments, the mixture of step (1) is kept at ambient temperature.

In some embodiments, the mixture of step (1) comprises from about 1.0 to 1.5 equivalents of maleic acid relative to the amount of Compound I in the mixture. In some embodiments, the mixture of step (1) comprises about 1.0, about 1.1, about 1.2, about 1.3, about 1.4, or about 1.5 equivalents of maleic acid relative to the amount of Compound I in the mixture.

In some embodiments, the mixture of step (1) comprises about 1.1 equivalents of maleic acid and about 6 volumes of a 5:1 mixture of isopropanol and MIBK, relative to the amount of Compound I in the mixture.

In some embodiments, the antisolvent in step (2) is methyl tert-butyl ether (MtBE), MIBK, water, heptane, or a combination thereof. In some embodiments, the antisolvent in step (2) is heptane. In some embodiments, from about 2 volumes to about 20 volumes of antisolvent relative to the amount of Compound I is added to the mixture in step (2). In some embodiments, from about 4 volumes to about 10 volumes of antisolvent relative to the amount of Compound I is added to the mixture in step (2). In some embodiments, the antisolvent is added in one portion. In other embodiments, the antisolvent is added in two or more portions.

In some embodiments, from about 4 volumes to about 10 volumes of heptane relative to the amount of Compound I is added to the mixture in step (2). In some embodiments, from about 4 volumes to about 10 volumes of heptane relative to the amount of Compound I is added to the mixture in step (2) over two portions.

In some embodiments, the amount of seed crystals of Pattern D added to the mixture in step (2) is about 0.05%, about 0.10%, about 0.15%, about 0.20%, about 0.25%, about 0.30%, about 0.35%, about 0.40%, about 0.45%, about 0.50%, about 0.60%, about 0.70%, about 0.80%, about 0.90%, or about 1.00%, relative to the amount of Compound I in the mixture. In some embodiments, the seed crystals are added in one portion. In other embodiments, the seed crystals are added in two or more portions.

In some embodiments, the mixture is heated to a temperature of from about 40° C. to about 50° C. in step (3). In some embodiments, the mixture is heated to a temperature of about 40° C., about 41° C., about 42° C., about 43° C., about 44° C., about 45° C., about 46° C., about 47° C., about 48° C., about 49, or about 50° C. in step (3).

In some embodiments, the mixture is heated for at least 8 h in step (3). In some embodiments, the mixture is heated for at least 8 h, at least 12 h, at least 18 h, or more in step (3). In some embodiments, the mixture is heated to a temperature of about 50° C. for about 8 h, about 12 h, or about 18 h in step (3).

In some embodiments, the mixture is heated to a temperature of about 50° C. for about 18 h in step (3).

In some embodiments, the slurry is cooled to ambient temperature in step (4). In some embodiments, the slurry is cooled to below ambient temperature in step (4). In some embodiments, the slurry is cooled to a temperature of not more than 25° C. in step (4). In some embodiments, the slurry is cooled to a temperature of from about 0° C. to about 20° C. in step (4). In some embodiments, the slurry is cooled to a temperature of about 20° C. in step (4).

In some embodiments, the slurry is cooled at a rate of at most 10° C./min in step (4). In some embodiments, the slurry is cooled at a rate of at most 2.5, 5.0, 7.5, or 10° C./min in step (4). In some embodiments, the slurry is cooled at a rate of at most 2.5° C./min in step (4). In some embodiments, the slurry is cooled at a rate of about 2.5° C./min in step (4).

In some embodiments, the slurry is cooled over at least 15 min in step (4). In some embodiments, the slurry is cooled over about 15 min, about 30 min, about 45 min, about 60 min, or more in step (4). In some embodiments, the slurry is cooled over about 15 min, about 30 min, about 45 min, about 60 min, about 90 min, or about 120 min in step (4). In some embodiments, the slurry is cooled over about 45 min in step (4).

In some embodiments, the slurry is cooled to a temperature of about 20° C. at a rate of at most 2.5° C./min in step (4). In some embodiments, the slurry is cooled to a temperature of about 20° C. over about 15 min, about 30 min, about 45 min, about 60 min or more in step (4). In some embodiments, the slurry is cooled to a temperature of about 20° C. over about 45 min in step (4).

In some embodiments, the solid obtained after filtration in step (5) is further dried. In some embodiments, the solid obtained after filtration in step (5) is dried under vacuum. In some embodiments, the solid obtained after filtration in step (5) is dried in a vacuum oven. In some embodiments, the solid obtained after filtration in step (5) is dried under vacuum at ambient temperature. In some embodiments, the solid obtained after filtration in step (5) is dried under vacuum at about 50° C. In some embodiments, the solid obtained after filtration in step (5) is Crystalline Pattern D of Compound I maleate.

### Recrystallization of Crystalline Pattern D of Compound I Maleate

In some embodiments, Crystalline Pattern D of Compound I maleate is crystallized directly from the reaction mixture prior to isolation.

In other embodiments, Crystalline Pattern D of Compound I maleate is recrystallized.

In some embodiments, Crystalline Pattern D of Compound I maleate is isolated and recrystallized.

In some embodiments, recrystallizing the Crystalline Pattern D of Compound I maleate comprises:
i. contacting Compound I maleate Pattern D with a suitable solvent to obtain a mixture;
ii. heating the mixture of step (i) to obtain a solution;
iii. seeding the solution with crystals of Pattern D of Compound I maleate to obtain a mixture;
iv. adding a suitable antisolvent to the mixture over a suitable amount of time;
v. heating the mixture for a suitable amount of time to obtain a slurry;
vi. cooling the slurry at a suitable cooling rate; and
vii. filtering the slurry to obtain Crystalline Pattern D of Compound I maleate.

In some embodiments, the suitable solvent in step (i) is ethanol, isopropanol, acetone, methyl acetate, or a combination thereof. In some embodiments, the suitable solvent in step (i) is ethanol, isopropanol, or methyl acetate, or a combination thereof. In some embodiments, the suitable solvent in step (i) is isopropanol.

In some embodiments, from about 2 volumes to about 20 volumes of solvent is used in step (i), relative to the amount of Compound I in the mixture. In some embodiments, from about 4 volumes to about 10 volumes of solvent is used in step (i), relative to the amount of Compound I in the mixture. In some embodiments, about 4, about 5, about 6, about 7, about 8, about 9, or about 10 volumes of solvent is used in step (i), relative to the amount of Compound I in the mixture.

In some embodiments, the suitable solvent in step (i) is ethanol, isopropanol, acetone, methyl acetate, or a combination thereof; and wherein from about 4 volumes to about 10 volumes of solvent is used in step (i), relative to the amount of Compound I in the mixture. The process of claim 46, wherein about 4 volumes of isopropanol is used in step (i), relative to the amount of Compound I in the mixture.

In some embodiments, the mixture is heated to a temperature of from about 30° C. to about 50° C. in step (ii). In some embodiments, the mixture is heated to a temperature of about 30° C., about 35° C., about 40° C., about 45° C., or about 50° C. in step (ii). In some embodiments, the process further comprises cooling the solution obtained in step (ii) to a temperature of about 30° C. prior to the seeding of step (iii). In some embodiments, the process further comprises cooling the solution obtained in step (ii) to a temperature of about 30° C. over about 2 hours prior to the seeding of step (iii).

In some embodiments, the amount of seed crystals of Pattern D added to the mixture in step (iii) is about 0.25%, about 0.50%, about 0.75%, about 1.0%, about 1.25%, about 1.50%, about 1.75%, about 2.0%, about 2.25%, about 2.5%, about 2.75%, about 3.0%, about 3.2%, about 3.5%, about 3.75%, about 4.0%, 4.25%, about 4.5%, about 4.75%, or about 5.0%, relative to the amount of Compound I in the mixture. In some embodiments, the seed crystals used in step (iii) are dry.

In some embodiments, the suitable antisolvent of step (iv) is MtBE, heptane, or a combination thereof. In some embodiments, the suitable antisolvent of step (iv) is heptane. In other embodiments, the suitable antisolvent of step (iv) is MtBE. In some embodiments, the antisolvent (e.g., MtBE) is added slowly to the reaction mixture in step (iv). In some embodiments, the antisolvent (e.g., MtBE) is added over at least 1 h, 2 h, at least 4 h, at least 6 h, or more in step (iv). In some embodiments, the antisolvent is added in one portion. In other embodiments, the antisolvent is added in two or more portions.

In some embodiments, the suitable antisolvent of step (iv) is MtBE, heptane, or a combination thereof; and wherein from about 3 volumes to about 10 volumes of solvent is used in step (iii), relative to the amount of Compound I in the mixture. In some embodiments, the suitable antisolvent of step (iv) is MtBE; and wherein the MtBE is added over at least 2 h, at least 4 h, at least 6 h, or more.

In some embodiments, in step (v) the mixture is heated to about 40° C., about 45°, or about 50° C. to obtain a slurry. In some embodiments, the mixture is heated for about 1 h, about 2, about 3 h, or more to obtain a slurry. In some embodiments, in step (v) the mixture is heated to about 40° C. for about 1 h, about 2 h, or about 3 h.

In some embodiments, the slurry obtained in step (v) is cooled to ambient temperature in step (vi). In some embodiments, the slurry is cooled to below ambient temperature in step (vi). In some embodiments, the slurry is cooled to a temperature of not more than 25° C. in step (vi). In some embodiments, the slurry is cooled to a temperature of from about 0° C. to about 20° C. in step (vi). In some embodiments, the slurry is cooled to a temperature of about 20° C. in step (vi).

In some embodiments, the slurry obtained in step (v) is cooled at a rate of at most 10° C./min in step (vi). In some embodiments, the slurry is cooled at a rate of at most 2.5, 5.0, 7.5, or 10° C./min in step (vi). In some embodiments, the slurry is cooled at a rate of at most 2.5° C./min in step (vi). In some embodiments, the slurry is cooled at a rate of about 2.5° C./min in step (vi).

In some embodiments, the slurry obtained in step (v) is cooled over at least 15 min in step (vi). In some embodiments, the slurry is cooled over about 30 min, about 45 min, about 60 min, about 90 min, about 120 min, or more in step (vi). In some embodiments, the slurry is cooled over about 30 min, about 45 min, about 60 min, about 90 min, or about 120 min in step (vi). In some embodiments, the slurry is cooled over about 120 min in step (vi).

In some embodiments, the slurry obtained in step (v) is cooled to a temperature of about 20° C. at a rate of at most 2.5° C./min in step (vi). In some embodiments, the slurry obtained in step (v) is cooled to a temperature of about 20° C. over about 30 min, about 60 min, about 90 min, about 120 min, or more in step (vi). In some embodiments, the slurry obtained in step (v) is cooled to a temperature of about 20° C. over about 120 min in step (vi).

In some embodiments, the cooled slurry of step (vi) is maintained at the cooled temperature for at least 2 h, at least, 3 h, at least 4 h, or more prior to step (vii). In some embodiments, the cooled slurry of step (vi) is maintained at about 20° C. for at least 2 h, at least, 3 h, at least 4 h, or more prior to step (vii). In some embodiments, the cooled slurry of step (vi) is maintained at about 20° C. for about 4 h prior to step (vii).

In some embodiments, the solid obtained after filtration in step (vii) is further dried. In some embodiments, the solid obtained after filtration in step (vii) is dried under vacuum. In some embodiments, the solid obtained after filtration in step (vii) is dried in a vacuum oven. In some embodiments, the solid obtained after filtration in step (vii) is dried under vacuum at ambient temperature. In some embodiments, the solid obtained after filtration in step (vii) is dried under vacuum at about 50° C. In some embodiments, the solid obtained after filtration in step (vii) is Crystalline Pattern D of Compound I maleate.

In some embodiments, the crystalline Pattern D of Compound I maleate as isolated shows no evidence of other forms, as determined by, for example, XRPD.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zrich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, Compound I is prepared as a salt with maleic acid or mandelic acid. In some embodiments, Compound I is prepared as a maleate salt or a mandelate salt. In some embodiments, Compound I is prepared as a maleate salt. In other embodiments, Compound I is prepared as a mandelate salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound I, comprise an organic solvent(s). In some embodiments, compositions comprising Compound I include a residual amount of an organic solvent(s). In some embodiments, compositions comprising Compound I comprise a residual amount of a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from ethyl acetate, isopropyl acetate, tert-butylmethylether, heptane, isopropanol, and ethanol.

In some embodiments, the compositions comprising Compound I include a detectable amount of an organic solvent. In some embodiments, the organic solvent is a Class 3 solvent.

In other embodiments are compositions comprising Compound I wherein the composition comprises a detectable amount of solvent that is less than about 1%, wherein the solvent is selected from acetone, 1,2-dimethoxyethane, acetonitrile, ethyl acetate, tetrahydrofuran, methanol, ethanol, heptane, and 2-propanol. In a further embodiment are compositions comprising Compound I wherein the composition comprises a detectable amount of solvent which is less than about 5000 ppm. In yet a further embodiment are compositions comprising Compound I, wherein the detectable amount of solvent is less than about 5000 ppm, less than about 4000 ppm, less than about 3000 ppm, less than about 2000 ppm, less than about 1000 ppm, less than about 500 ppm, or less than about 100 ppm.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “about” a number refers to that number plus or minus 10% of that number. The term “about” a range refers to that range minus 10% of its lowest value and plus 10% of its greatest value.

The term “moiety” refers to a specific segment or functional group of a molecule. Chemical moieties are often recognized chemical entities embedded in or appended to a molecule.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compound and solid state forms described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action.

### Methods of Dosing and Treatment Regimens

Cushing's syndrome is a rare disorder characterized by chronic, excess glucocorticoid exposure. Clinical signs of Cushing's syndrome include growth of fat pads (collarbone, back of neck, face and trunk), excessive sweating, dilation of capillaries, thinning of the skin, muscle weakness, hirsutism, depression/anxiety, hypertension, osteoporosis, insulin resistance, hyperglycemia, heart disease, and a range of other metabolic disturbances resulting in high morbidity. If inadequately controlled in its severe forms, Cushing's syndrome is associated with high mortality. Although glucocorticoid excess can sometimes be ACTH independent, for example from excessive autonomous secretion of cortisol from a hyperfunctioning adrenal adenoma, carcinoma, or steroid abuse, about 60-80% of all cases are ACTH dependent Cushing's syndrome, known as Cushing's disease. Cushing's disease is caused by microadenomas of pituitary corticotropic cells that secrete excess ACTH. Corticotroph adenomas are small, usually slow growing, benign tumors that normally come to clinical attention as a result of the effects of glucocorticoid excess, rather than because of the physical effects of an expanding tumor. First line treatments for Cushing's disease are surgical and involve removal of either the ACTH-secreting tumor in the pituitary or the adrenal glands themselves. As surgery is often unsuccessful, contraindicated, or delayed, medical therapy for these patients becomes necessary.

Current treatment options include inhibitors of steroid synthesis enzymes that can prevent the production of cortisol and improve symptoms, but these treatments also induce a host of unwanted side effects due to the accumulation of other steroid products. In one aspect, an MC2R antagonist is used in the treatment of Cushing's syndrome. In some embodiments, an MC2R antagonist is used in the treatment of Cushing's disease. In some embodiments, glucocorticoid excess is ACTH independent. In some embodiments, glucocorticoid excess is ACTH dependent.

Ectopic ACTH syndrome, or ectopic Cushing's syndrome or disease, is essentially the same as Cushing's disease, except that the underlying tumor expressing ACTH is outside the pituitary gland. In some embodiments, the tumors are small carcinoid tumors that occur anywhere in the lungs or gastrointestinal tract. In some embodiments, an MC2R antagonist is used in the treatment of ectopic ACTH syndrome.

Congenital adrenal hyperplasia (CAH) is characterized by a reduction or loss of cortisol synthesis and excessive ACTH and corticotropin-releasing hormone. CAH can result from a variety of genetic defects in the adrenal steroidal biosynthesis pathway. In some embodiments, CAH is due to a mutation in 210-hydroxylase. The lack of cortisol removes the negative feedback to the pituitary which leads to excessive ACTH secretion. The resulting excessive adrenal stimulation causes overproduction of steroid precursors which also have negative consequences (e.g., hyperandrogenism). Administration of replacement glucocorticoids typically does not adequately suppress ACTH without also causing Cushing's-like symptoms. In some embodiments, an MC2R antagonist is used in the treatment of CAH.

In addition to Cushing's disease and CAH it has also been hypothesized that there might be a role for an MC2R antagonist in the treatment of depressive illness and septic shock. In some embodiments, an MC2R antagonist compound is used in the treatment of depressive illness. In some embodiments, an MC2R antagonist is used in the treatment of septic shock.

In one embodiment, the compound and solid state forms described herein are used in the preparation of medicaments for the treatment of diseases or conditions in a mammal that would benefit from modulation of MC2R receptor activity, including, but not limited to, Cushing's diseases, Cushing's syndrome, ectopic ACTH syndrome (EAS), and congenital adrenal hyperplasia (CAH). Methods for treating any of the diseases or conditions described herein in a mammal in need of such treatment, involves administration of pharmaceutical compositions that include at least one compound disclosed herein or a pharmaceutically acceptable salt, active metabolite, prodrug, or pharmaceutically acceptable solvate thereof, in therapeutically effective amounts to said mammal.

In any of the aforementioned aspects are further embodiments in which the effective amount of the compound disclosed herein, or a pharmaceutically acceptable salt thereof, is: (a) systemically administered to the mammal; and/or (b) administered orally to the mammal.

### EXAMPLES

### Abbreviations

ACN or MeCN: acetonitrile;
Am.: amorphous;
Aq or aq: aqueous;
Compound I: N-[(3S)-1-azabicyclo[2.2.2]octan-3-yl]-6-(2-ethoxyphenyl)-3 [(2R)-2-ethyl-4-[1-(trifluoromethyl)-cyclobutanecarbonyl]piperazin-1-yl]pyridine-2-carboxamide;
DEE: diethyl ether;
DMSO: dimethylsulfoxide;
DSC: differential scanning calorimetry;
DVS: dynamic vapor sorption;
Et: ethyl;
EtOAc: ethyl acetate;
EtOH: ethanol;
equiv or eq.: equivalents;
h or hr: hour;
hrs: hours;
FPLC: high-performance liquid chromatography;
TPA: isopropyl alcohol;
IPAc: isopropyl acetate;
M: molar;
MEK: methyl ethyl ketone;
Me: methyl;
MeOAC: methyl acetate;
MeOH: methanol;
MIBK: methyl isobutyl ketone;
mins or min: minutes;
MtBE: methyl tert-butyl ether;
NMR: nuclear magnetic resonance;
RH: relative humidity;
rt or RT: room temperature;
SCXRD: single crystal x-ray diffraction;
TGA: thermogravimetric analysis;
THF: tetrahydrofuran;
vol: volume, typically used for reaction volume or ratio of solvents;
w/w: weight ratio; and
XRPD: X-ray powder diffraction.

The following examples are provided for illustrative purposes only and not to limit the scope of the claims provided herein.

### Example 1. Preparation of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I)

The preparation of Compound I has been previously described (e.g., see Example 31 of WO 2019/236699, and U.S. Pat. No. 10,562,884, which is incorporated by reference for such methods of making Compound I).

### Example 2. Salt Screening

Salt screenings of Compound I was set up with 19 counter ions using 1.1 equivalents of counter ion in all cases except sulfuric acid where 0.55 equivalents were used. In addition, 2.2 equivalents of HCl was also explored.

Experiments were set up for the salt screening process in 2 mL vials containing 5 mm stir bars). A stock solution of Compound I was prepared in anhydrous EtOH (50 mg/mL). Stock solutions of the counter ions were also prepared in EtOH. Compound I (20 mg, approximately 380 μL stock solution) and 1.1 equivalents of the counter ions were added to each vial at room temperature. Upon addition, transition from a colorless or pale-yellow to bright yellow solution was observed with the following counter ions: hydrobromic acid, nitric acid, hydrochloric acid, methanesulfonic acid, sulfuric acid, and toluenesulfonic acid. Each of these counter ions have a negative pKa. Vials were then sealed and allowed to stir briefly (<2 h). Solvent was evaporated at room temperature under a steady flow of nitrogen and then put under vacuum at room temperature overnight for thorough drying. The resultant solids were beige to yellow gels in most cases with some vials appearing to have a thin film of solid on the vial walls.

Approximately 10 volumes of solvent (200 μL) was added to each vial for screening. The first two solvents selected were MtBE and MIBK. Once solvents were added, the mixtures (or solutions) were allowed to stir overnight (400 RPM). In vials where the gel was not broken by solvent addition, sonication and vortexing of the vial was employed to some success.

Most experiments in MIBK resulted in solutions while experiments in MtBE typically resulted in gums or gels. However, two experiments formed slurries appropriate for sampling. Flowable yellow powders were isolated from experiments with 2 eq. of HCl. Following sampling, the first round of solvents were evaporated under a steady stream of nitrogen and dried under vacuum over the weekend. Diethyl ether and IPA:heptane (2:8 vol.) were selected as the second round solvents with 10 volumes of the respective solvent being dispensed per experiment.

In a second round of counter ion screening, the first two solvent systems explored were diethyl ether and IPA:heptane (2:8 vol.). 20 volumes of diethyl ether and 10 volumes of the IPA:heptane mixture were used. Through the first round of solvent screening with these counter ions, most experiments resulted in gums or gels. However, five slurries suitable for filtration were observed. Following sampling, the first round of solvents were evaporated under nitrogen and dried under vacuum overnight. The second round of solvents selected were EtOH:DEE (2:8 vol.) and ACN:MtBE (1:9 vol.). After 10 volumes of solvent was added, sonication was employed to enable free stirring and the mixtures were allowed to stir at room temperature overnight. Most of the experiments resulted in gels or gumming with only one slurry appropriate for filtration.

XRPD analysis is typically done in three stages: 1) Wet: XRPD of the wet cake is done for all samples whenever solids appropriate for sampling were observed. 2) Dry: Unique solids are then left on XRPD plates and dried under vacuum at 50° C. for at least 3 h. XRPD of unique dry solids are then collected. 3) Humid: Solids are then exposed to >95% relative humidity overnight and XRPD on resulting solids is collected. The humid environment is generated by placing a beaker of saturated potassium sulfate in water in a sealed container. This procedure is not carried out for any amorphous solids obtained. All XRPD patterns are compared to counter ion XRPD patterns and known free molecule patterns.

No solids were collected with the following counterions: oxalic acid, methanesulfonic acid, sulfuric acid, succinic acid, toluenesulfonic acid; in the following solvents: tert-butyl methylether (MtBE), diethyl ether. Methyl isobutyl ketone (MIBK), and 2-propanol (IPA):heptane (2:8).

No solids were collected with the following counterions: hydrobromic acid, nitric acid, tartaric acid; in the following solvents: tert-butyl methylether (MtBE), methyl isobutyl ketone (MIBK), and 2-propanol (IPA):heptane (2:8).

No solids were collected with the following counterions: hydrochloric acid (1.1 and 2.2 equivalents); in the following solvents: methyl isobutyl ketone (MIBK), and 2-propanol (IPA):heptane (2:8).

No solids were collected with the following counterions: benzenesulfonic acid, gentisic acid; in the following solvents: diethyl ether, IPA:diethyl ether, IPA:heptane (2:8), ACN:MtBE (1:9).

No solids were collected with the following counterions: malic acid; in the following solvents: diethyl ether, EtOH:diethyl ether, IPA:heptane (2:8), ACN:MtBE (1:9).

No solids were collected with the following counterions: citric acid, fumaric acid, phosphoric acid, glucoronic acid; in the following solvents: IPA:diethyl ether, IPA:heptane (2:8), ACN:MtBE (1:9).

Salt screening experiments resulted in gumming in most experiments but experiments with HBr, nitric acid, HCl (both 1 and 2 eq.), tartaric acid, maleic acid, citric acid, fumaric acid, phosphoric acid, mandelic acid, glucuronic acid, and aspartic acid provided slurries suitable for filtration.

Further results of the salt screening experiments are tabulated in the following table.

TABLE 1
Salt Screening of Compound I

| | | XRPD | | |
|---|---|---|---|---|
| Counter ion | Solvent | Wet | Dry | Humid |
| Hydrobromic Acid | diethyl ether | Amorphous | | |
| Nitric Acid | diethyl ether | Amorphous | | |
| Hydrochloric Acid | MtBE | | | |
| (1.1 equiv) | diethyl ether | Amorphous | | |
| Hydrochloric Acid | MtBE | Amorphous | | |
| (2.2 equiv) | diethyl ether | Amorphous | | |
| Maleic Acid | MtBE | Amorphous | | |
| | diethyl ether | A | | |
| | MIBK | | | |
| | IPA:heptane (2:8) | B | C | C |
| Tartaric Acid | diethyl ether | Amorphous | | |
| Citric Acid | diethyl ether | Amorphous | | |
| Fumaric Acid | diethyl ether | Amorphous | | |
| Phosphoric Acid | diethyl ether | Amorphous | | |
| Mandelic Acid | diethyl ether | | | |
| | IPA:diethyl ether | | | |
| | IPA:heptane (2:8) | E | E | E |
| | ACN:MtBE (1:9) | | | |
| Glucoronic Acid | diethyl ether | Amorphous | | |
| Aspartic Acid | diethyl ether | | | |
| | EtOH:diethyl ether | Aspartic acid | | |
| | IPA:heptane (2:8) | | | |
| | ACN:MtBE (1:9) | | | |

Note:
A blank cell indicates that no solids were collected.

Amorphous solids were recovered from experiments with HBr, nitric acid, HCl (both 1 and 2 eq.), tartaric acid, citric acid, fumaric acid, phosphoric acid, and glucuronic acid. Only the counter ion itself was recovered from the experiment with aspartic acid.

A low crystalline tacky solid was isolated from diethyl ether in an experiment with maleic acid designated Pattern A. A flowable white crystalline powder was isolated from IPA:heptane (2:8 vol.), also in an experiment with maleic acid, and appeared to be a mixture of forms that were designated Patterns B+C.

Maleate Pattern B mostly converts to Pattern C upon drying (a small amount of residual Pattern B was present by XRPD). Pattern C was also stable with no observed deliquescence after exposure to >95% relative humidity overnight.

Following filtration of the maleate, a small amount of beige gum was left in the vial. An additional 10 volumes of IPA:heptane (2:8 vol.) was added to the vial and it was left to stir over the weekend yielding a flowable white slurry with no gum being visible. Upon filtration and drying Pattern C was isolated again as a flowable white crystalline powder. The NMR of the maleate Pattern C shows reasonable agreement with the parent structure.

A crystalline pattern was confirmed by XRPD for an experiment with mandelic acid, denoted Pattern E. The solid had a slightly tacky texture immediately after filtration. The mandelate proved stable to drying and no deliquescence or loss of crystallinity was observed after exposure to >90% relative humidity overnight.

### Example 3. Small Scale Preparation of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide mandelate (Compound I Mandelate)

Approximately 60 mg of Compound I was weighed in a 4 mL vial with a 10 mm stir bar. Stock solution in EtOH of mandelic acid (20 mg/mL) was added to and stirred for 1 h. The solvent was evaporated under a stream of nitrogen and the beige gel dried under vacuum at room temperature overnight.

Following drying of the beige gel, 10 volumes of IPA:heptane (2:8 vol.) were added to the vial. The mixture was sonicated and vortexed to enable free stirring. A small amount of crystalline Compound I mandelate (from Example 2) was added as seed. After several hours, only a small amount of free flowing solid was observed that was confirmed to be amorphous by XRPD. The majority of the solid was a beige gum. An additional 10 volumes of IPA:heptane (2:8 vol.) was added followed by sonication and vortexing. After approximately one hour, a flowable slurry was observed that was allowed to stir overnight. The slurry was sampled the following day for XRPD, which demonstrated the previously observed crystalline Pattern E.

### Example 4. Small Scale Preparation of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I Maleate)

Approximately 60 mg of Compound I was weighed in a 4 mL vial with a 10 mm stir bar. Stock solution in EtOH of maleic acid (20 mg/mL) was added to and stirred for 1 h. The solvent was evaporated under a stream of nitrogen and the beige gel dried under vacuum at room temperature overnight.

Following drying of the beige gel, 10 volumes of IPA:heptane (2:8 vol.) were added to the vial. The mixture was sonicated and vortexed to enable free stirring. A small amount of crystalline Compound I maleate (from Example 2) was added as seed. After several hours, no free flowing solid was observed and a beige oily gum was visible on the vial walls. The mixture was sonicated periodically and allowed to stir overnight at room temperature. No change was observed. At this stage, 50 μL of MIBK was added followed by brief sonication. Within 2 h a thin slurry was observed, which was allowed to continue stirring overnight at room temperature. After filtration, the crystalline pattern that was observed was a mixture of two patterns that were designated Patterns B and C. While the solid isolated from the screening experiments in Example 2 contained mostly Pattern C, the solid isolated in this scale up experiment was mostly form Pattern B.

### Example 5. Preparation of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide mandelate (Compound I mandelate)

Approximately 200 mg of Compound I and 55 mg (1.1 eq.) of mandelic acid were combined in a 20 mL vial with a stir bar. Initially, five volumes of IPA were added with stirring at room temperature, which quickly resulted in dissolution and a pale-yellow solution. The solution was seeded with crystalline Pattern E of Compound I mandelate (from Example 2) resulting in a hazy solution. The seed appeared to be retained. Heptane was slowly added dropwise with stirring until a mixture of IPA:heptane (1:1 vol.; 10 vol. total) was attained. The hazy solution gradually thickened into a white slurry during heptane addition. An hour after addition a thick gel-like slurry was observed. The slurry was sonicated and allowed to stir at room temperature over the weekend resulting in a thick white slurry that no longer appeared gellike. The slurry was filtered and the vial was washed with 3 volumes of IPA:heptane (2:8 vol.). The white solid was dried under vacuum (−30 inHg) at room temperature overnight.

The yield of the mandelate was 78 mg (31% w/w). The mandelate was confirmed to be Pattern E by XRPD. The NMR spectrum indicated the presence of 1.1 wt. % residual IPA, 0.13 wt. % heptane, and a Compound I: counter ion ratio of 1.00:1.07.

### Example 6. Preparation of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I Maleate)

Approximately 200 mg of Compound I and 42 mg (1.1 eq.) of maleic acid were combined in a 20 mL vial with a stir bar. Initially, 5 volumes of IPA and 25 μL MIBK were added with stirring at room temperature. This resulted in gumming that gradually dissolved to yield a pale-yellow solution. The solution was seeded with crystalline patterns B+C Compound I maleate (from Example 2) but the seed was not retained. Heptane was slowly added dropwise with stirring until a mixture of IPA:heptane (5:7 vol.) was attained, which resulted in the solid precipitating as a gum. To address the gumming, 1 vol. of MIBK was added in 7 additional aliquots of 25 μL each. The final solvent composition was IPA:heptane:MIBK (5:7:1 vol.; 13 vol. total). The gum was sonicated for approximately 5 min and appeared to be breaking up yielding some flowable solids. After 1 h, a flowable white slurry was observed. The slurry was allowed to stir at room temperature over the weekend, which did not result in a change in slurry rheology. The slurry was filtered and the vial was washed with 3 volumes of IPA:heptane (2:8 vol.). The white solid was dried under vacuum (−30 inHg) at room temperature overnight.

The yield of the maleate was 126 mg (52% w/w). The wet cake of the maleate was confirmed to be a mixture of Pattern B+Pattern C by XRPD. After drying, conversion to Pattern C, with no residual Pattern B, was observed. The NMR spectrum indicated the presence of 0.08 wt. % residual heptane and a Compound I:counter ion ratio of 1.00:0.87. A qualitive humidity exposure test was performed by exposing approximately 10 mg of the maleate to 80% RH overnight. No detectable mass gain was observed.

### Example 7. Preparation of Crystalline Pattern C of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I Maleate)

Amorphous Compound 1(49.9 mg) was weighed into a 2 mL vial. Maleic acid (1.1 eq., 11.5 mg) was weighed into the vial. Approximately 250 μL (5 vol.) of MIBK and a 5 mm stir bar were added to the vial. The material was stirred into solution at room temperature. The solution was seeded with a spatula tip of Crystalline Pattern C. The solution became slightly cloudy, indicating the seed was retained. Three volumes of heptane antisolvent was added slowly, 30 μL every 5 min until a total of 150 μL was added, with vortexing between each 30 μL addition. Some gumming was observed during heptane addition, which appeared as yellowish spots stuck to the vial walls. After 3 vol. heptane was added, the resulting slurry was moved to a hot plate to stir at 50° C. and the yellowish gum was broken up with a spatula. The slurry was stirred at 50° C. for 30 min. An additional 6 vol. of heptane was added (50 μL every 5 min for 30 min, or 300 μL total) dropwise with vortexing between each addition. The slurry was then stirred at 50° C. for 1 h at which point a small amount of yellow gum on the vial walls was observed and the slurry had poor flowability. Sonication was also employed at this stage but did not lead to an improvement in slurry rheology. An additional 5 vol. (250 μL) heptane was added (total 14 vol. added) and the slurry was allowed to stir overnight at 50° C. The following day, the slurry was moved to room temperature and stirred for 1 h, although the rheology and flowability remained poor. Small chunks of orange gum were observed on the vial walls. A small sample of the slurry was filtered and plated for XRPD, which was indicative of Pattern C.

### Example 8. Alternative Preparation of Crystalline Pattern C of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I Maleate)

Compound I freebase (6.15 g; 5.72 g accounting for solvent content) and maleic acid (1.20 g, 1.1 molar equivalents (eq.)) were dissolved in 6 vol. (34.2 mL) of IPA:MIBK (5:1 vol.) with stirring and warming to 30° C. The brown-red solution was cooled to 25° C. and addition of heptane began at a rate of 0.35 mL/min. After the addition of 4 vol. (22.5 mL) of heptane, the solution was seeded with maleate Pattern B+C (15 mg, 0.26 wt. %). The seed was retained, and the mixture began to precipitate oily-gel-like brown solids as heptane addition continued. At ca. 6 vol. heptane addition, the mixture continued to thicken, precipitating oily-gummy-gel-like solids. After the addition of the full 10 vol. (57.2 mL) of heptane, the rheology of the mixture appeared to have improved and some suspended solid particles were visible in addition to the gummy-jelly brown mixture and crust. A sample was drawn for analysis by microscopy, showing some small irregular particles and gel-gum. The mixture was sonicated for 3-5 min, further improving the mixture rheology and another sample of the slurry was analyzed by microscopy. The mixture was warmed to 50° C. and left to stir overnight.

The next day, the resultant pale-brown slurry was sampled for microscopy and cooled to 15° C. A sample was drawn for XRPD analysis at this time and it exhibited a mixture of Pattern B and Pattern C. The solids were collected by filtration and rinsed thrice with 1 vol. IPA:heptane (2:8 vol.). The cake retained the mother liquor and hence was manually mixed with a spatula. An additional volume of heptane was added twice, to mix with the clay-like brown solids and generate a powdery solid. A sample of the powder was analyzed by XRPD and exhibited Pattern C. The remainder was transferred to a vial and dried in a vacuum oven at 50° C., 10-2-10-1 Torr for 18 h. Pattern C was recovered (5.8 g, 84% yield).

Crystalline Pattern C of Compound I Maleate was used as the input material for the subsequent polymorph screening experiments.

### Example 9. Preparation of Crystalline Pattern D of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I Maleate)

Approximately 50.1 mg of amorphous Compound I was weighed into a 4 mL vial. 11.7 mg (1.1 eq.) of maleic acid was weighed into the vial with weigh paper. IPA:MIBK (5:1 vol.) (300 μL, 6 vol.) and a 10 mm stir bar were added and the material was stirred into solution. Heptane (50 μL, 1 vol.) was added dropwise every 5 min for 15 min until 150 μL (3 vol.) was added. The solution was then seeded with Crystalline Pattern B. The solution became slightly cloudy, indicating the seed was retained. Heptane was added immediately after seeding, continuing at 1 vol. every 5 min dropwise until 9 vol. (450 μL) was added. Some gumming was observed on the vial wall after 6 vol. heptane was added. After 9 vol. was added a thick slurry had formed with gum visible on the vial wall. Upon sonication of the slurry for 5 min and stirring at 50° C. for 1 h, the gum had lessened slightly. A small sample of the slurry was filtered and plated for XRPD at this point, Pattern B was observed.

The slurry was allowed to stir for another hour at 50° C., after which the gum had mostly broken and the slurry appeared more flowable. A small sample of the slurry was filtered and plated for XRPD at this point, Pattern B was observed again. The slurry was allowed to stir at 50° C. overnight, after which no gum was visible and the flowability of the slurry appeared to have improved. A small sample of the slurry was filtered and plated for XRPD, which was indicative of a new pattern, designated Pattern D.

### Example 10. Alternative Preparation of Crystalline Pattern D of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I maleate)

About 200 mg of Compound I Maleate Pattern C (from Example 8) was loaded into 4 mL vials. Two volumes of solvent (IPA or MeOAc) was added, and the mixtures stirred at 50° C. to dissolve. The solutions were cooled to 40° C. and seeded with Pattern D. The seed appeared to be retained, dispersing to a fine silty-haze in the red-brown solutions. The mixtures were cooled by 2.5° C. every 10 min until reaching 25° C., then all heating ceased (RT ˜22-24° C.), and the mixtures stirred overnight (18 h). The following day, both slurries were sampled for microscopy and XRPD.

The slurry in IPA was thinner than in MeOAc, showed a mixture of needle and platelike crystals, and exhibited Pattern D by XRPD. The solids were collected and the vial rinsed once with 1 vol. IPA to collect all remaining solids and rinse the cake. The solids were dried in a vacuum oven at 50° C., 10⁻²-10⁻¹ Torr for 6 h then weighed for yield (26 mg, 13%).

The slurry in MeOAc was thicker than in IPA, showed plate-like crystals, and exhibited Pattern D by XRPD. The solids were collected and the vial rinsed once with 1 vol. MeOAc to collect all remaining solids and rinse the cake. The solids were dried in a vacuum oven at 50° C., 10⁻²-10⁻¹ Torr for 6 h then weighed for yield (74 mg, 37%). The dried solids were analyzed by DSC and TGA/DSC.

### Example 11. Larger Scale Preparation of Crystalline Pattern D of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I Maleate)

A process adapted from previous experiments was performed to convert the Compound I free base to the maleate, Pattern D according to the following steps:
1. The freebase, 12.03 g (11.19 g accounting for solvent content) was weighed into a 400 mL EasyMax vessel.
2. Maleic acid (2.35 g, 1.1 equivalents (eq.)) was added to the vessel.
3. IPA (˜5 vol., 60 mL) and 1 vol. of methyl isobutyl ketone (MIBK, 12 mL) were added at room temperature (RT) producing a dark brown solution.
4. Stirring was set to 150 rpm.
5. Heptane (4 vol., 48 mL) was added over 1.5 h.
6. Seeded with 30 mg of Pattern D.
   a. Seed was retained.
7. Heptane (2 vol., 24 mL) was added over 45 min.
   a. Thin slurry with a darker precipitate collected on the bottom of the vessel, potentially oiling out.
   b. Seeded with an additional 15 mg Pattern D.
   c. Some gumming was observed at this stage.
   d. Increased stirring to 300 rpm.
8. Heptane (4 vol., 48 mL) was added over 1.5 h.
   a. Significant gumming observed during and following addition.
   b. Vessel was briefly sonicated (˜3 min).
9. Heated to 50° C. over 30 min.
10. Held overnight at 50° C.
11. Flowable light-brown slurry was obtained with minimal gumming or solids on vessel walls.
   a. XRPD sampling confirmed only Pattern D was present.
12. Cooled to 20° C. over 45 min.
13. Held for 1 h at 20° C.
14. Filtered, washed with 5 mL heptane (twice), and dried over at least 2 days at RT under vacuum (−27.5 inHg).
15. Yielded a flowable light-brown powder (yield=10.9 g, 81% mol/mol).

XRPD confirmed only Pattern D was present in the product.

### Example 12. Recrystallization of Crystalline Pattern D of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide maleate (Compound I Maleate)

Solubility measurements (Example 13) identified favorable profiles for four solvents (EtOH, IPA, acetone, and MeOAc) and two antisolvents (MtBE and heptane) that were carried on to small scale recrystallization experiments. During the small-scale antisolvent recrystallizations, issues with gumming, oiling out, or form conversion were observed in all experiments with heptane. EtOH:MtBE and IPA:MtBE were identified as the best solvent systems to pursue at 2-3 g scale.

The crystallization of Compound I Maleate Pattern D required long induction periods accommodated by ensuring adequate hold times coupled with slow and controlled antisolvent addition to mitigate issues with gumming, oiling, and precipitation of amorphous solids. Furthermore, filtrations conducted in atmosphere when RH was high (>55%) led to dissolution of the wet cake on the filtration apparatus.

In spite of these unpredictable challenges a suitable and reliable process was developed for the recrystallization of Compound I Maleate Pattern D. The optimized process is as follows:
1. Add solid to vessel.
2. Add IPA (4 vol.) at RT.
3. Set stirring to accommodate ˜0.7 W/kg mixing energy based on final process volume.
4. Heat to 50° C. or until dissolution.
5. Cool to 30° C. over 2 h.
6. Seed with ˜2% seed loading (dry, Pattern D).
7. Hold for 30 min.
8. Add MtBE (3 vol.) over 1.5 h.
9. Hold for 3 h.
10. Add MtBE (7 vol.) over 6 h.
11. Hold for 1 h.
12. Heat to 40° C. over 30 min.
   a. Set stirring to accommodate ˜0.18 W/kg mixing energy based on final process volume.
13. Hold for 2.5 h.
14. Cool to 20° C. over 2 h.
15. Hold for 4 h.
16. Filter under nitrogen, ensure the cake has deliquored.
17. Wash with 2×2 vol. of IPA:MtBE (3:7 vol.).
18. Ensure adequate drying conditions (−30 inHg vacuum, 50° C., nitrogen bleed).

Purity for processes in this solvent system (IPA:MtBE) are consistently high with the most representative process having a purity of 99.68% a/a. The yield for this process was demonstrated to be ˜90% w/w. The isolated solids display a high degree of crystallinity. A temperature cycling step was added following antisolvent addition to improve crystallinity. A controlled filtration procedure is critical and should be performed under inert atmosphere to ensure the cake has fully deliquored prior to drying.

### Example 13. Solubility Assessment of Solid State Forms of Compound I

The solubility of certain solid state forms of Compound I was determined in various solvents by either gravimetric solubility or solvent addition.

### Gravimetric Solubility Determination

About 25 mg of compound was weighed into vials. Approximately 5 vol. (0.125 mL) of solvent was added to the solids and the mixtures stirred at the designated temperature (RT or 50° C.). Solutions were noted and an additional 0.125 mL of solvent was added to the slurries. If a thin slurry was noted at this stage, no additional solvent was added. If a thicker slurry was noted, 0.25 mL of solvent was added (total volume 0.5 mL). If a thick slurry was again noted, an additional 0.25 mL of solvent was added (total volume 0.75 mL). After stirring for 2 days, the slurries were centrifuged, the supernatant was collected into preweighed vials, and the solvent evaporated at 50° C. (ambient pressure) without stirring. The vials containing the solid residues were dried in a vacuum oven at 50 C, 10⁻²-10⁻¹ Torr, for 3 h before weighing again to determine the mass of solid that was dissolved in the supernatant (i.e. mg solid/mL solvent).

### Solvent Addition Solubility Determination

About 20 mg of compound was weighed into vials. Solvent was added in aliquots and stirred at the designated temperature until complete dissolution was observed. This measurement gave a solubility range. Results from the solubility experiments are shown in the following table.

TABLE 2
Solubility of Solid State Forms of Compound I at Room Temperature and 50° C.

| | Solubility (mg/mL) | | | | |
|---|---|---|---|---|---|
| | Solid State Form | | | | |
| | Amorphous | Pattern C | | Pattern D | |
| | free base | Maleate | | Maleate | |
| | Temperature | | | | |
| Solvent | RT | RT | 50° C. | RT | 50° C. |
| cyclohexane | | <3 | 3 | | |
| n-heptane | <26 | <3 | 3 | <3^{c} | <3^{c} |
| diethyl ether | | | | 4^{c} | |
| toluene | >500^{a} | <3 | 7 | | |
| MeOH | | >200^{a} | | >50^{a} | |
| EtOH | >500^{a} | >200^{a} | | >50^{a} | |
| IPA | | >200^{a} | >500^{a} | 68^{c} | |
| acetone | >500^{a} | >200^{a} | | >50^{a} | |
| MIBK | >500^{a} | 49 | 71 | 43^{c} | 65^{c} |
| MtBE | >500^{a} | 4 | 4 | <3^{c} | <3^{c} |
| THF | | >200^{a} | | >50^{a} | |
| EtOAc | >500^{a} | 21 | 34 | 43^{d} | 68^{d} |
| IPAc | | 8 | 13 | 7^{c} | 13^{c} |
| ACN | | >200^{a} | | | |
| chloroform | | >200^{a} | | | |
| DMSO | | >200^{a} | | | |
| MEK | | >200^{a} | | >50^{a} | |
| MeOAc | | >200^{a} | >500^{a} | 168^{c} | >250^{a, c} |
| IPA:water (1:1 vol.) | 167-250^{b} | | | | |
| IPA:water (2:8 vol.) | | >200^{a} | | | |
| IPA:water (9:1 vol.) | | | | >333^{a} | |
| acetone:water (2:8 vol.) | | >200^{a} | | | |
| ACN:water (2:8 vol.) | | >200^{a} | | | |
| MeOAc:IPAc (1:1 vol.) | | | 37-45 | | |
| acetone:heptane (1:1 vol.) | | | 31-37^{b} | | |
| IPA:heptane (1:1 vol.) | | | 16-19 | | |
| IPA:MtBE (1:1 vol.) | | | 36-43 | 10 | |
| IPA:MtBE (3:7 vol.) | | | | 7 | |
| IPA:MtBE (1:9 vol.) | | | | <3 | |
| EtOH:MtBE (3:7 vol.) | | | | 51 | |
| EtOH:MtBE (1:9 vol.) | | | | 4 | |
| MeOAc:MtBE (1:1 vol.) | | | | 17 | |
| MeOAc:MtBE (3:7 vol.) | | | | 7 | |
| MeOAc:MtBE (1:9 vol.) | | | | 3 | |

^{a}compounds were freely soluble at the tested concentration;
^{b}generated oil with solvent addition, solubility was determined to be the volume at which the mixture became homogenous;
^{c}XRPD of isolated solids indicated that compound remained Pattern D;
^{d}XRPD of isolated solids indicated that compound converted to Pattern C

### Results:

Experiments conducted with Compound I (amorphous free base) resulted in complete dissolution for all solvents tested except heptane and IPA:water (1:1 vol.), indicating that the compound is freely soluble. Oiling was observed after two volumes of IPA:water (1:1 vol.) was added, which cleared with the addition of more solvent. Due to low solubility in heptane, the solids were allowed to stir overnight in ca. 35-40 volumes of solvent, but the compound gummed to the walls of the vial in this time.

Experiments conducted with Pattern C of Compound I Maleate indicated that solids were freely soluble in MeOH, EtOH, acetone, THF, ACN, MEK, DMSO, and in water containing systems at 8:2 vol. water:organic compositions. Gels/gums were generated in water and 95:5 vol. water:organic systems.

Pattern D of Compound I Maleate was freely soluble and soluble in EtOAc and MIBK at RT and 50° C. Pattern D was soluble in IP at RT and a slurry could not be maintained at 50° C. Pattern D was insoluble in heptane, diethyl ether, and MtBE. Solids obtained from the experiments with Pattern D were also sampled for XRPD. All experiments remained Pattern D, except those in EtOAc were Pattern C was present at RT and became the dominant pattern at 50° C. Solubility of Pattern D was also conducted in mixtures of selected solvents (EtOH, IPA, and MeOAc with MtBE cosolvent).

### Example 14. Polymorph Screening 2: Short-Term Slurries

### Example 14a. Slurries of Pattern C in Various Solvents

Short-term slurries with Compound I Maleate Pattern C were carried out in nine solvents at two temperatures (RT and 50° C.). The slurries were stirred at the specified temperature for 2 days followed by XRPD analysis of the solids. A summary of the results to the short-term slurry experiments is presented in the following table.

TABLE 3
Results from Short-term Slurry Experiments
of Pattern C at RT and 50° C.

Slurry XRPD

| Solvent | RT | 50° C. |
|---|---|---|
| cyclohexane | C | C |
| n-heptane | C | C |
| toluene | C + B (wet) | D + C + trace B |
| | C (dry) | (wet and dry) |
| IPA | D | |
| MIBK | B + C (wet) | D |
| | C + trace B (dry) | |
| MtBE | C | C |
| EtOAc | C | C |
| IPAc | C | C |
| water | | |
| MeOAc | D | |

A set of slurries in organic:water (5:95 vol.) mixtures were prepared at RT. Organic solvents tested were EtOH, IPA, THF, acetone, and ACN. All five of these cosolvent samples generated brown gel and oil. After stirring for 5 h, the temperature was raised to 40° C. and stirring continued overnight. No change was observed in these mixtures, yellowish solution and brown gum persisted.

### Example 14b. Additional Slurries of Patterns C and D

Additional slurries were set up with Compound I Maleate Pattern C and Pattern D in selected solvents. Conditions (solvent, volumes, temperature) and results are shown in the following table.

TABLE 4
Results from Additional Slurry Experiments of Patterns C and D

| Input | | | |
|---|---|---|---|
| Pattern | Solvent System | Temperature | Slurry XRPD |
| C | IPA:MIBK:n-heptane | RT | B + C (2 days) |
| | (5:1:10 vol.) | | C + trace B (5 days) |
| | | 50° C. | B + C (2 days) |
| | | | D (5 days) |
| C | IPA | 10° C. | amorphous (16 h) |
| C | MeOAc | 10° C. | B + trace C (16 h) |
| C | MeOAc (water saturated) | RT | — |
| C | IPAc | 50° C. | C (2 days) |
| | | | C (5 days) |
| D | MIBK | RT | D (2 days) |
| | | | D (5 days) |
| C | MIBK:MtBE (1:1 vol.) | 50° C. | amorphous (4 days) |
| C | MIBK:heptane (1:1 vol.) | 50° C. | C (4 days) |

Results: In 5 vol. neat MeOAc, Pattern C fully dissolved, then Pattern D precipitated after stirring for 3 days. In 4 vol. of water saturated MeOAc, Pattern C dissolved and remained a solution. The solids recovered from MeOAc at 10+C were analyzed as a wet cake, after sitting at ambient conditions for 1 h, then after exposure to >95% RH for 16 h. The solids converted to Pattern C, no evidence of reversion to Pattern B was observed.

### Example 14c. Amorphous Slurries

Glass-like amorphous solid was made by evaporation of a solution of Crystalline Pattern C of Compound I Maleate in 5 vol. of MeOH. The solution was distributed equally among 10 vials, then evaporation done at 50° C. overnight without stirring. The gels were dried in a vacuum oven at 50° C., 10⁻²-10⁻¹ Torr for 3 h before the slurry solvent was added and the mixtures stirred at RT. A summary of results is shown in the following Table.

TABLE 5
Results from Short-term Slurry Experiments
of Amorphous Compound I Maleate

| Solvent System | Slurry XRPD* |
|---|---|
| tolene | L.C. + broad peaks (5 days) |
| diethyl ether | — |
| MtBE | — |
| EtOAc | C (1 day) |
| IPAc | A (2 days) |
| MIBK | — |
| MIBK:n-heptane (1:1 vol.) | A + B (wet) |
| | C + B + trace A (dry) (5 days) |
| IPA:n-heptane (1:1 vol.) | B + trace C (2 days) |
| IPA:MIBK:n-heptane (5:1:10 vol.) | B + trace C (2 days) |
| EtOAc (water saturated) | — |

*L.C. means low crystallinity.
Hyphens indicate insufficient solids for XRPD analysis after 5 days.

Results: Most of the samples in which the solids did not fully dissolve had started to precipitate a very fine quantity of solids suspended in solution at the 4-h mark, but there were insufficient quantities for analysis at that time. All mixtures were left stirring at RT overnight. After stirring for 24 h, the fine haze in EtOAc had turned into a medium-thin slurry and was collected for analysis, exhibiting Pattern C. The rest of the samples were still solution (water saturated EtOAc and MIBK) or mostly glassy solids with a very small quantity of suspended solids in slurry (rest), and continued to stir at RT. The mixtures were monitored over the next 5 days and the slurry solids collected and analyzed by XRPD when sufficient amounts appeared present for analysis.

The mixture of Pattern A and Pattern B collected from MIBK:heptane (1:1 vol.) was left on the bench at ambient conditions for three days then analyzed by XRPD. The diffractogram showed an increase in peaks associated with Pattern C, and an apparent loss of crystallinity. Upon further drying in a vacuum oven at 50° C., 10⁻²-10⁻¹ Torr for 20 h, the peaks corresponding to Pattern C became the dominant features of the XRPD diffractogram.

### Example 15. Polymorph Screening 3: Evaporative Crystallizations

After stirring for 2 days, the Compound I Maleate slurries generated for gravimetric solubility determination (Example 12) were centrifuged, the supernatant was collected into preweighed vials, and the solvent was evaporated at 50° C. (ambient pressure) without stirring. The vials containing the solid residues were dried in a vacuum oven at 50° C., 10⁻²-10⁻¹ Torr, for 3 h. Most of the solids recovered from evaporation were dry and powdery. Minimal quantities of gels/glass-like solids were obtained from evaporation of antisolvents. Results from the evaporative crystallization studies are summarized in the following table.

TABLE 6
Results from Evaporative Crystallization Experiments

| | |
|---|---|
| Solubility | XRPD* |

| | | |
|---|---|---|
| (mg/mL) | Undersaturated | Saturated |

| Solvent | RT | 50° C. | Solution | Solution |
|---|---|---|---|---|
| cyclohexane | <3 | 3 | — | — |
| n-heptane | <3 | 3 | — | — |
| MIBK | 49 | 71 | C | D |
| MtBE | 4 | 4 | — | — |
| THF | >200 | | B + C (L.C.) | — |
| EtOAc | 21 | 34 | C | C |
| IPAc | 8 | 13 | C | C |
| MeOH | >200 | | amorphous | — |
| EtOH | >200 | | — | — |
| acetone | >200 | | — | — |
| MEK | >200 | | — | — |

*L.C. means low crystallinity.
Hyphens indicate insufficient solids for XRPD analysis after 5 days.

Results: The solution generated in THF at RT slowly evaporated while stirring despite parafilm being used to seal the vial cap. This left an opaque brown residue that was analyzed by XRPD. The solutions generated in MeOH, EtOH, acetone, and MEK at RT were put in a freezer at −20° C. for 1.5 weeks. When no solids precipitated, the caps were removed, and the red-brown solutions were placed at 50° C. to evaporate the solvent without stirring. The resultant red-brown gels were dried in a vacuum oven at 50° C., 10⁻²-10⁻¹ Torr, for 3 h. These solids were probed with a needle and it was observed that the solids from evaporation of MeOH were the most brittle and glass-like. A sample of the solids from evaporation of MeOH showed an amorphous pattern by XRPD.

### Example 16. Polymorph Screening 4: Cooling Crystallizations

### Example 16a. Cooling in Neat Solvents

About 23 mg of Compound I Maleate Pattern C was weighed into 2 mL vials and dissolved in the selected solvent at 50° C. Cooling was done with two regimes. Fast-cooling was done by immersing the vial containing the 50° C. solution in an ice bath and leaving the vials undisturbed. Slow-cooling was done at a rate of 5° C./h from 50° C. to RT with stirring. Results from the cooling crystallization studies are summarized in the following table.

TABLE 7
Results from Cooling Crystallization Experiments in Neat Solvent

| Solvent | Amt [volumes] | Procedure | XRPD* |
|---|---|---|---|
| MIBK | 14 | Fast-cooling | L.C.; broad peaks |
| EtOAc | 40 | Fast-cooling | — |
| MeOAc | 2 | Fast-cooling | L.C.; broad peaks |
| IPA | 2 | Fast-cooling | amorphous |
| MeOH | 5 | Fast-cooling | — |
| EtOH | 5 | Fast-cooling | — |
| acetone | 5 | Fast-cooling | — |
| ACN | 5 | Fast-cooling | — |
| MIBK | 14 | Slow-cooling | B + D |
| EtOAc | 40 | Slow-cooling | — |
| MeOAc | 2 | Slow-cooling | D |
| IPA | 2 | Slow-cooling | D |

*L.C. means low crystallinity.
Hyphens indicate insufficient solids for XRPD analysis.

Results: The fast-cooling experiments generated gel-like slurries within a couple hours in the ice bath, except in EtOAc. The XRPD patterns exhibited by these jelly solids were very low crystalline with broad peaks. Some minimal fine brown solids were observed suspended in the EtOAc solution but were insufficient for collection and analysis. Hence, the mixture was moved to −20° C. in an attempt to increase the quantity of precipitated solids.

A homogeneous slurry was only generated in MeOAc during the slow-cooling experiment. Precipitation occurred around 40° C. and thickened on further cooling. Pattern D was collected from MeOAc. Significant crusting was observed in the EtOAc and MIBK samples, potentially affecting the results of these experiments due to the influence of evaporation and seeding with the crust. XRPD analysis was not feasible due to the minimal quantity of both crust and the fine hazy solids in EtOAc. After stirring at RT for some time, some gel-like solids began to appear stuck to the vial below the solvent level in MIBK, and all the solids were mixed and collected, exhibiting a mixture of Pattern D and Pattern B by XRPD. The IPA solution initially cooled to a viscous brown-red solution. Precipitation from the IPA solution after stirring for 2 days at RT yielded a small quantity of Pattern D.

### Example 16b. Cooling in Solvent/Antisolvent Mixtures

About 20 mg of Compound I Maleate Pattern C was loaded into vials. Solvent mixtures were prepared in 1:1 vol. composition. Solvent was added in aliquots, with stirring, at 50° C. until the solids dissolved. When the solids had dissolved, cooling was done at a rate of 5° C./h. Except for MIBK mixtures, these were moved to another hot plate to continue stirring at 50° C. (See, Example 14b). Results from the cooling crystallization experiments are summarized in the following table.

TABLE 8
Results from Cooling Crystallization Experiments
in Solvent/Antisolvent Mixtures

| Solvent Mixture (1:1 vol.) | Amt [volumes] | XRPD* |
|---|---|---|
| MeOAc:IPAc | 27 | C + B |
| IPA:MtBE | 28 | Amorphous |
| MIBK:MtBE | 67 | — |
| acetone:heptane | 32 | — |
| IPA:heptane | 62 | — |
| MIBK:heptane | 67 | — |

*Hyphens indicate insufficient solids for XRPD analysis.

Results: Some crusting was noted in the vial containing the MeOAc/IPAc solution at the end of cooling. Brown gel/gum was noted in the acetone/heptane mixture. None of the samples were slurries upon reaching RT, and were left to stir overnight. The MeOAc/IPAc mixture yielded some gellike solids in solution in addition to the gel. This was mixed and collected for analysis, showing Pattern C+B. The IPA/heptane mixture had generated some very fine brown solids suspended in solution and an attempt was made to collect these for XRPD analysis, but the solids were trapped in the filter paper and none of the material could be recovered for analysis. The acetone/heptane and IPA/MtBE mixtures were moved to 10° C. and stirred. The IPA/MtBE sample generated a gel-like slurry, but the combination of poor filtration and warming of the sample upon isolation yielded very little material to analyze. No peaks were observed.

### Example 17. Polymorph Screening 5: Antisolvent Crystallizations

About 23 mg of Compound I Maleate Pattern C was weighed into vials. The solids were dissolved in the chosen solvent at RT. Antisolvent addition was performed by one of two methods, either the reverse or direct addition method.

For reverse antisolvent addition, the solution was transferred all at once to four times the solvent volume of antisolvent with rapid stirring. For example, if solids dissolved in 0.5 mL solvent then the solution was added at once to 2.0 mL antisolvent with vigorous stirring.

For direct antisolvent addition, twice the volume of solvent was used for the antisolvent, and this was added in four portions, dropwise, over one hour. The mixtures were stirred during the antisolvent addition. For example, if solids dissolved in 0.5 mL solvent then 1.0 mL antisolvent was added over 60 min. The mixtures were warmed to 40° C. and stirred for 16 h, followed by cooling to RT and stirring an additional 24 h before samples were collected.

Results for the antisolvent crystallization experiments are shown in the following tables.

TABLE 9
Results of the Reverse Antisolvent Crystallizations

| | Amt | Anti- | Amt | |
|---|---|---|---|---|
| Solvent | [volumes] | solvent | [volumes] | XRPD* |
| IPA:water | 5 | water | 20 | — |
| (8:2 vol.) | | | | |
| IPA | 5 | MtBE | 20 | L.C., broad peaks |
| IPA | 5 | heptane | 20 | L.C., broad peaks |
| acetone:water | 5 | water | 20 | — |
| (8:2 vol.) | | | | |
| acetone | 5 | MtBE | 20 | L.C., broad peaks |
| acetone | 5 | heptane | 20 | C + B |
| MIBK | 25 | MtBE | 100 | L.C., broad peaks |
| MIBK | 25 | heptane | 100 | L.C., broad peaks |
| IPA:MIBK | 6 | heptane | 24 | L.C., broad peaks + |
| (5:1 vol.) | | | | C (L.C.) |
| THF | 5 | MtBE | 20 | L.C., broad peaks |

*L.C. means low crystallinity.
Hyphens indicate insufficient solids for XRPD analysis.

TABLE 10
Results of the Direct Antisolvent Crystallizations

| | Amt | Anti- | Amt | |
|---|---|---|---|---|
| Solvent | [volumes] | solvent | [volumes] | XRPD* |
| IPA:water | 5 | water | 10 | — |
| (8:2 vol.) | | | | |
| IPA | 5 | MtBE | 10 | D (L.C.) |
| IPA | 5 | heptane | 10 | C + trace D + |
| | | | | trace B |
| acetone:water | 5 | water | 10 | — |
| (8:2 vol.) | | | | |
| acetone | 5 | MtBE | 10 | L.C., broad peaks |
| acetone | 5 | heptane | 10 | D + C + B |
| MIBK | 25 | MtBE | 50 | L.C., broad peaks |
| MIBK | 25 | heptane | 50 | B |
| IPA:MIBK | 6 | heptane | 12 | B + C |
| (5:1 vol.) | | | | |
| THF | 5 | MtBE | 10 | L.C., broad peaks |

*L.C. means low crystallinity.
Hyphens indicate insufficient solids for XRPD analysis.

Results: All of the samples that generated a slurry upon mixing the solution with the antisolvent in the reverse antisolvent mode produced very low crystalline solids with broad peaks and remained unchanged upon drying

The results at completion of direct antisolvent addition were noted as being very similar to the reverse antisolvent addition experiments, which yielded gel-like, very low crystalline solids.

Some of the samples generated in the antisolvent crystallization experiments were mixtures of Pattern B, Pattern C, and Pattern D. These samples were left at ambient conditions for one week, then analyzed again by XRPD. Pattern B converted to Pattern C. However, no observable conversion between C and D was noted.

### Example 18. Polymorph Screening 6: Milling

About 30 mg of Compound I Maleate Pattern C was loaded into a milling capsule and one volume of solvent (if any) was added, along with a ¼″ steel ball as milling media. The solid was milled with a Wig-L-bug at 3500 rpm for 30 s, then collected and analyzed by XRPD. A summary of results is given in the following table.

TABLE 11
Results of the Milling Experiment

| Solvent | XRPD* |
|---|---|
| none (dry milling) | amorphous |
| water | amorphous |
| MtBE | C (low crystallinity) |
| n-heptane | C |
| EtOAc | C + B |

### Example 19. One-Week Stability of Crystalline Forms

Approximately 10 mg of the Crystalline Pattern D of Compound I Maleate, 20 mg of the Crystalline Pattern C of Compound I Maleate, and 10 mg of the Crystalline Pattern E of Compound I Mandelate were weighed into open 4 mL vials that were placed into a 20 mL vial containing a saturated NaCl solution. The vial was sealed and kept at 40° C. for 7 days, creating an atmosphere at 75% relative humidity in the system. After 7 days, the materials were sampled and plated for XRPD analysis. No visible change was observed by XRPD after one-week under those conditions for any of the three tested crystalline patterns. HPLC purity analysis confirmed no degradation of the compound was observed for any of the crystalline patterns.

The stability test was repeated on Patterns C and D of Compound I Maleate for 11 days. No visible change was observed by XRPD after 11 days for either Pattern, and HPLC purity analysis confirmed no degradation of the compound was observed.

### Example 20. Stability of Pattern B and Conversion to Pattern C

Pure Pattern B was generated in the antisolvent crystallization with MIBK and heptane. After the sample had been sitting at ambient conditions for one week, the sample was analyzed by XRPD again, showing the presence of some low intensity peaks associated with Pattern C. The solids were scraped from the XRPD plate and used for simultaneous TGA/DSC analysis. The DSC thermogram showed a melting endotherm with an onset of 120° C., and this appeared to coincide with a stepwise mass loss of 1.55% in the TGA thermogram. The TGA thermogram had a number of weight loss events (separated by analyzing the derivative of the TGA curve). After the solids required for the TGA/DSC analysis were transferred to the pan, the remainder of the solids were re-pressed to the XRPD sample plate and analyzed again. After scraping and replating, the solids had changed significantly, showing peaks associated with Pattern C at a greater intensity than those associated with Pattern B.

### Example 21. Competitive Slurries with Compound I Maleate

### Example 21a. Competitive Slurries in IPA:Heptane, MIBK, and EtOAc

Saturated solutions were made in IPA:heptane (1:1 vol.), MIBK, and EtOAc at RT and 50° C. using Compound I Maleate Pattern C as input. A saturated solution was also generated in IPA:MIBK:heptane (5:1:10 vol.) at 37° C. In separate vials, about 12 mg of Pattern C and 6 mg of Pattern D were combined. The saturated solutions (supernatant separated from the slurry solids) were added to the vials and these continued to stir at the same temperature that the saturated solution was formed. The slurries were checked to ensure homogeneity. After stirring for 30 min, the slurries were sampled, and the solids analyzed by XRPD. Although no Pattern B had been added, all of the slurries showed significant quantities of Pattern B, except in EtOAc.

After stirring for 3 days, the slurries were sampled again. Those stirring at 50° C. had converged to Pattern D (IPA:heptane (1:1 vol.) and MIBK) or Pattern C (EtOAc). Those at lower temperatures were still mixtures of all three Patterns (IPA:heptane (1:1 vol.) and IPA:MIBK:heptane (5:1:10 vol.)) or of Pattern C and Pattern D (MIBK and EtOAc).

The slurries were sampled again after stirring for 6 days. Slurries showing a single pattern were unchanged from the 3-day timepoint, but mixtures of D, C, and B continued to change. In IPA:MIBK:heptane (5:1:10 vol.) at 35° C., the trend appeared to favor Pattern B and C, as relative proportions B increased gradually over time. The slurries were allowed to stir at their respective temperatures.

The competitive slurries were sampled again after 13 days. The room temperature EtOAc sample was absorbed by filter paper, IPA:MIBK:heptane (5:1:10 vol.)) at 35° C. was crusted out of the solvent, and EtOAc at 50° C. had dried out completely. Other trials showed minor crusting. The results were unchanged from the 6-day timepoint, with the only significant change occurring with the slurry in IPA:heptane (1:1 vol.), in which Pattern C was no longer present. Data from competitive slurries is summarized in the following table.

TABLE 12
Results from the Competitive Slurry Experiments
with Compound I Maleate Pattern C and Pattern D

| | Temp. | XRPD* | | | |
|---|---|---|---|---|---|
| Solvent | [° C.] | 0.5 h | 3 days | 6 days | 13 days |
| IPA:heptane (1:1 vol.) | RT | D + B + C | D + B^{a} + C^{b} | D + B^{b} + C^{a} | D + B |
| MIBK | RT | D + B + C | D + C | Mostly | Mostly |
| | | | | D + C + D | D + C + D |
| EtOAc | RT | C + trace D | C + trace D | C + trace D | — |
| IPA:MIBK:heptane | 35 | D + C + B | D + C^{b} + B | D^{a} + C + B^{b} | — |
| (5:1:10 vol.) | | | | | |
| IPA:heptane (1:1 vol.) | 50 | D + B | D | D | D |
| MIBK | 50 | D + C + B | D | D | D |
| EtOAc | 50 | C | C | C | — |

*Hyphens indicate no data collected;
^{a}decreasing;
^{b}increasing.

Results: Competitive slurry results show that conversion to Pattern D occurs at elevated temperature when seeds of Pattern D are present. However, at lower temperature and in some solvent systems the conversion appears to be slow. In the room temperature competitive slurries, the relative quantity of D appears to remain stable over time, but B/C did not readily convert to D within the sampling timeframe.

### Example 21b. Competitive Slurries in IPA:MIBK, MIRK, and IPAc

Competitive slurries were set up with maleate Patterns B, C, and D. Vials (4 mL) were set up with 10 mm stir bars and 700 μL for three solvent systems at two temperatures: 22-25° C. and 50° C. Maleate Pattern D was added to all experiment vials by tip of spatula (˜3-5 mg) until a thin slurry had formed. The slurries were allowed to stir at their respective temperatures for 1 h. The slurries were then syringe filtered into new 4 mL vials with 0.45 μm filters. These new vials contained preweighed quantities of seed material with a mixture of maleate Patterns B/C and D. As Patterns B and C appeared to be in equal representation in dry solid, twice as much of this material was preweighed as Pattern D to achieve a relatively similar proportion of each pattern in the resulting slurry. These slurries were allowed to stir at their respective temperatures for 1 h, and were then sampled for XRPD analysis.

As some crusting was observed, the vials were vortexed and set up in a manner to prevent crust formation on the heated vial walls above the liquid level. The slurries were then sampled for XRPD analysis at the 4-day and 7-day mark. Data from competitive slurries is summarized in the following table.

TABLE 13
Results from the Competitive Slurry Experiments
with Compound I Maleate Patterns B, C, and D

| | Temperature | XRPD* | | |
|---|---|---|---|---|
| Solvent | [° C.] | 1 h | 4 days | 7 days |
| MIBK:IPA:heptane | 50 | D | D | D |
| (0.2:1.0:3.0 vol.) | | | | |
| MIBK | 50 | D | D | D |
| IPAc | 50 | C + D | C + B^{a} | C + B |
| MIBK:IPA:heptane | RT | C + D | C + D | C + D + B |
| (0.2:1.0:3.0 vol.) | | | | |
| MIBK | RT | D + C | D | D |
| IPAc | RT | C + D | C + D^{b} | C + D |

*Hyphens indicate no data collected;
^{a}trace amount;
^{b}decreasing.

Results: Pattern D was observed in MIBK:IPA:heptane (0.2:1.0:3.0 vol.) and neat MIBK at 50° C. at all timepoints. In MIBK at RT, only Pattern D was observed by the 4- and 7-day mark. In IPAc at RT, initial conversion from Pattern D to Pattern C was observed, but about the same relative amount of Pattern D was still observed at the 4- and 7-day mark. However, at 50° C. in IPAc, Pattern D was not observed at the 4-day mark and a mixture of B and C was observed at 4 and 7 days. MIBK:IPA:heptane at RT also appeared to be remained a mixture of B, C, and D but conversion was slower.

### Example 22. Solubility in Simulated Fluids and Water

Calibration samples for solubility in simulated fluids were prepared with freebase Compound I.

Solubility in simulated fluids were studied with the mandelate and maleate solids. The simulated fluids FaSSIF (fasted-state intestinal), FaSSGF (fasted-state gastric), and FeSSIF (fed-state intestinal) were removed from the refrigerator, warmed to room temperature, and the pH of each was measured. Between 1.9 and 2.0 mg of each salt was weighed into four vials and 1 mL of each fluid was added, including water. Stir bars (10 mm) were added to the vials and the vials were stirred at 37° C. After 30 mi (mandelate samples) or 1 h (maleate samples), the pH of each solution was measured, then 400 μL of each was removed and syringe filtered with 0.45 m syringe filters. Approximately 300 μL of the recovered filtrate was diluted 5× with diluent and injected for HPLC analysis. The remainder of the samples in the vials were allowed to stir overnight. After 24 h stirring at 37° C., samples were filtered and diluted as before, and injected for analysis. The resulting data is shown in the following table.

TABLE 14
Solubility in Simulated Fluids

| | | | Sample pH | | Solubility [mg/mL]* | |
|---|---|---|---|---|---|---|
| | | Initial | 30 min/ | | 30 min/ | |
| Salt Form | Fluid | pH | 1 h ^{a} | 24 h | 1 h ^{a} | 24 h |
| mandelate Pattern | FaSSIF | 6.35 | 6.41 | — | 1.19 | 1.38 |
| E | | | | | | |
| mandelate Pattern | FaSSGF | 1.49 | 1.53 | — | >1.69 | >1.74 |
| E | | | | | | |
| mandelate Pattern | FeSSIF | 4.83 | 4.89 | — | >2.20 | >2.13 |
| E | | | | | | |
| mandelate Pattern | water | — | — | — | 2.52 | >2.17 |
| E | | | | | | |
| maleate Pattern C | FaSSIF | 6.35 | 6.23 | 6.98 | 1.24 | 1.41 |
| maleate Pattern C | FaSSGF | 1.49 | 4.66 | 5.08 | <2.50 | >2.50 |
| maleate Pattern C | FeSSIF | 4.83 | 1.50 | 1.89 | >2.50 | >2.50 |
| maleate Pattern C | water | — | ~6.35 | 5.50 | >2.50 | >2.50 |
| maleate Pattern D | FaSSIF | 6.35 | 6.23 | 6.92 | 1.50 | 1.47 |
| maleate Pattern D | FaSSGF | 1.49 | 4.66 | 5.07 | >2.50 | >2.50 |
| maleate Pattern D | FeSSIF | 4.83 | 1.50 | 1.85 | >2.50 | >2.50 |
| maleate Pattern D | water | — | ~6.35 | 5.60 | >2.50 | >2.50 |

*solubility is greater than the listed value as material had dissolved completely.
Hyphens indicate no data collected.
^{a} mandelate samples taken at 30 min; maleate samples taken at 1 hr.

There are minor differences in the solubility of the maleate crystalline forms in FaSSIF. After 1 h, solubility of Pattern D was measured at 1.50 mg/mL and Pattern C at 1.24 mg/mL in FaSSIF. Solids recovered from these experiments after 24 h were oily and appeared as amorphous by XRPD.

### Example 23: X-Ray Powder Diffraction (XRPD)

Although the following diffractometer was used, other types of diffractometers could be used. Furthermore, other wavelengths could be used and converted to the Cu Kα.

“Characteristic peaks”, to the extent they exist, are a subset of observed peaks and are used to differentiate one crystalline polymorph from another crystalline polymorph (polymorphs being crystalline forms having the same chemical composition). Characteristic peaks are determined by evaluating which observed peaks, if any, are present in one crystalline polymorph of a compound against all other known crystalline polymorphs of that compound to within +0.2° 2-Theta.

XRPD was performed using a Bruker D8 Advance equipped with LYNXEYE detector in reflection mode (i.e. Bragg-Brentano geometry). Samples were prepared on Si zero-return wafers. The parameters for XRPD methods used are listed below:

| Parameter | Regular Scan | High Resolution Scan |
|---|---|---|
| X-ray wavelength | Cu Kα1, 1.540598 Å | Cu Kα1, 1.540598 Å |
| X-ray tube setting | 40 kV, 40 mA | 40 kV, 40 mA |
| slit condition | 0.6 mm div. + 2.5° soller | 0.6 mm div. + 2.5° soller |
| scan mode | step | step |
| scan range (°2θ) | 4-30 | 4-40 |
| step size (°2θ) | 0.03 | 0.02 |
| dwell time (s/step) | 0.23 | 0.9 |
| spin | yes (0.5 Hz) | yes (0.5 Hz) |

The 2-Theta peak values that are provided for the XRPD are within +0.2° 2-Theta.

### Characterization of Solid-State Forms of Compound I

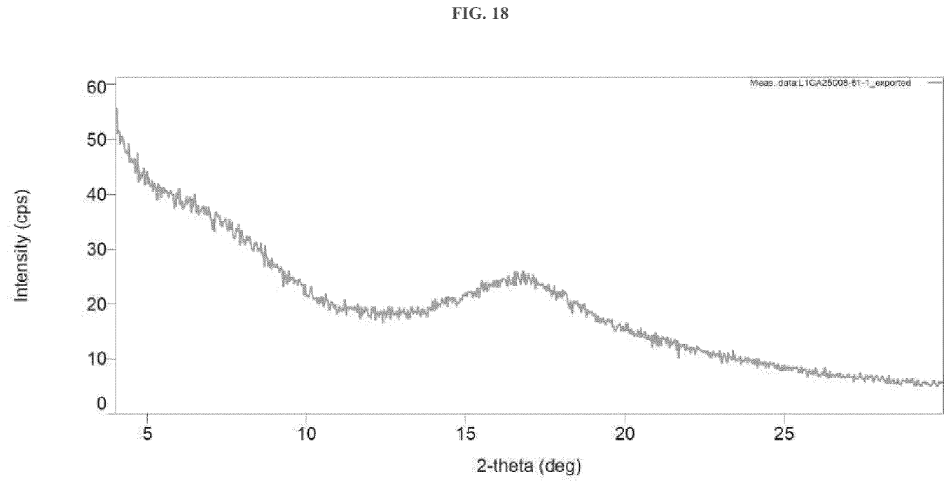

The X-Ray powder diffraction pattern for crystalline Pattern A of Compound I Maleate is displayed in FIG. 12. The X-Ray powder diffraction pattern for crystalline Pattern B of Compound I Maleate is displayed in FIG. 9. The X-Ray powder diffraction pattern for crystalline Pattern C of Compound I Maleate is displayed in FIG. 5. The X-Ray powder diffraction pattern for crystalline Pattern D of Compound I Maleate is displayed in FIG. 1. The X-Ray powder diffraction pattern for crystalline Pattern E of Compound I Mandelate is displayed in FIG. 13. The X-ray powder diffraction pattern for amorphous Compound I is displayed in FIG. 18.

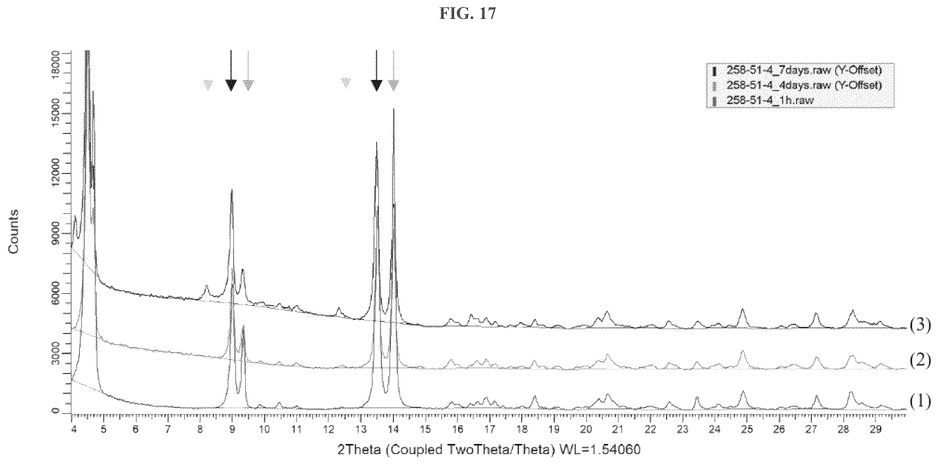

A comparison of the X-Ray powder diffraction patterns for crystalline Patterns B, C, and D of Compound I Maleate is displayed in FIG. 17, as measured during the competitive slurry experiment in Example 21. Lines (1) and (2) are mixtures of Patterns C+D; Line (3) is a mixture of Patterns B+C+D. Characteristic Pattern B peaks (orange arrow; 8.2±0.2° 2-Theta; 12.3±0.2° 2-Theta); Characteristic Pattern C peaks (black arrow; 9.0±0.2° 2-Theta; 13.5±0.2° 2-Theta); Characteristic Pattern D peaks (green arrow; 9.4±0.2° 2-Theta; 14.0±0.2° 2-Theta).

### Characterization of Crystalline Pattern A of Compound I Maleate

The X-Ray powder diffraction pattern for crystalline Pattern A of Compound I Maleate is displayed in FIG. 12. Characteristic XRPD peaks include: 4.2±0.2° 2-Theta, 8.3±0.2° 2-Theta, and 12.5±0.2° 2-Theta. A listing of XRPD peaks is shown in the following table:

| | Relative intensity* |
|---|---|
| Peak (°2θ) | (a.u.) |
| 4.2 | 100 |
| 4.4 | 6 |
| 8.3 | 23 |
| 12.5 | 15 |
| 13.5 | 3 |
| 16.7 | 7 |
| 20.9 | 3 |
| 29.4 | 3 |

### Characterization of Crystalline Pattern B of Compound I Maleate

The X-Ray powder diffraction pattern for crystalline Pattern B of Compound I Maleate is displayed in FIG. 9. Characteristic XRPD peaks include: 4.1±0.2° 2-Theta, 8.2±0.2° 2-Theta, 12.3±0.2° 2-Theta, and 16.4 0.2° 2-Theta. A listing of XRPD peaks is shown in the following table:

| | Relative intensity* |
|---|---|
| Peak (° 2θ) | (a.u.) |
| 4.1 | 100 |
| 8.2 | 25 |
| 12.3 | 14 |
| 16.4 | 13 |
| 20.6 | 5 |
| 28.9 | 4 |

### Characterization of Crystalline Pattern C of Compound I Maleate

The X-Ray powder diffraction pattern for crystalline Pattern C of Compound I Maleate is displayed in FIG. 5. Characteristic XRPD peaks include: 4.5±0.2° 2-Theta, 9.0±0.2° 2-Theta, 13.5±0.2° 2-Theta, and 18.4 0.2° 2-Theta. A listing of XRPD peaks is shown in the following table:

| | Relative intensity* |
|---|---|
| Peak (° 2θ) | (a.u.) |
| 4.5 | 100 |
| 9.0 | 23 |
| 9.8 | 6 |
| 10.5 | 7 |
| 13.5 | 34 |
| 18.4 | 14 |

### Characterization of Crystalline Pattern D of Compound I Maleate

The X-Ray powder diffraction pattern for crystalline Pattern D of Compound I Maleate is displayed in FIG. 1. Characteristic XRPD peaks include: 4.7±0.2° 2-Theta, 9.4±0.2° 2-Theta, 11.0±0.2° 2-Theta, and 14.0 0.2° 2-Theta. A listing of XRPD peaks is shown in the following table:

| | Relative intensity* |
|---|---|
| Peak (° 2θ) | (a.u.) |
| 4.7 | 77 |
| 9.4 | 24 |
| 9.9 | 20 |
| 11.0 | 21 |
| 11.1 | 13 |
| 12.4 | 15 |
| 14.0 | 100 |
| 15.8 | 22 |
| 16.6 | 22 |
| 16.9 | 11 |
| 17.2 | 15 |
| 18.7 | 15 |
| 19.2 | 16 |

### Characterization of Crystalline Pattern E of Compound I Mandelate

The X-Ray powder diffraction pattern for crystalline Pattern E of Compound I Mandelate is displayed in FIG. 13. Characteristic XRPD peaks include: 5.6±0.2° 2-Theta, 10.5±0.2° 2-Theta, 14.9±0.2° 2-Theta, and 16.5 0.2° 2-Theta. A listing of XRPD peaks is shown in the following table:

| | Relative intensity* |
|---|---|
| Peak (° 2θ) | (a.u.) |
| 5.6 | 33 |
| 10.5 | 85 |
| 10.8 | 62 |
| 11.4 | 64 |
| 11.7 | 23 |
| 11.9 | 39 |
| 13.9 | 59 |
| 14.9 | 76 |
| 16.5 | 93 |
| 17.8 | 43 |
| 18.3 | 56 |
| 19.1 | 86 |
| 19.2 | 68 |
| 20.4 | 100 |
| 20.6 | 64 |

In some embodiments, measurements on independently prepared samples on different instruments may lead to variability which is greater than +0.2° 2-Theta.

### Example 24: Differential Scanning Calorimetry (DSC)

Differential scanning calorimetry was performed using a Mettler Toledo DSC³⁺. The sample (1-5 mg) was weighed directly in a 40 μL hermetic aluminum pan with pin-hole and analyzed according to the parameters below:

| Method | Ramp |
|---|---|
| Sample size | 1-5 mg |
| Heating rate | 5.0 to 10.0° C./min |
| Temperature range | 30 to 200° C. |
| Method gas | N₂ at 60.00 mL/min |

### Characterization of Solid State Forms of Compound I

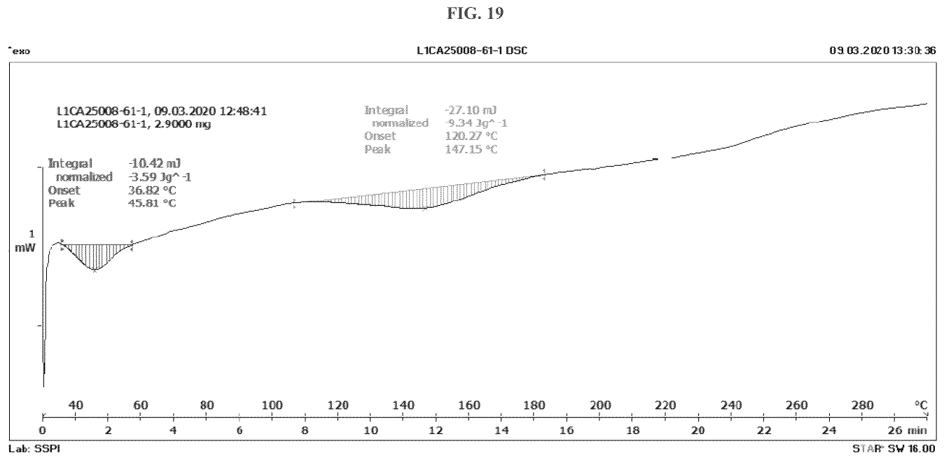

The DSC thermogram for crystalline Pattern B of Compound I Maleate is displayed in FIG. 10. The DSC thermogram for crystalline Pattern C of Compound I Maleate is displayed in FIG. 6. The DSC thermogram for crystalline Pattern D of Compound I Maleate is displayed in FIG. 2. The DSC thermogram for crystalline Pattern E of Compound I Mandelate is displayed in FIG. 14. The DSC thermogram for amorphous Compound I is displayed in FIG. 19.

Differential Scanning Calorimetry (DSC) thermogram thermal events for the solid state forms are as described in the following table:

| Salt Form | Pattern | DSC Thermal Events |
|---|---|---|
| Free base | amorphous | two broad endotherms having: an onset at |
| | | 36.8° C. and a peak at 45.8° C.; and an |
| | | onset at 120.3° C. and a peak at 147.2° C. |
| Maleate | Pattern B | endotherm having an onset at 141.4° C. and |
| | | peak at 150.5° C. |
| | Pattern C | endotherm having an onset at 144.1° C. and |
| | | peak at 150.7° C. |
| | Pattern D | endotherm having an onset at 161.6° C. and |
| | | peak at 168.4° C. |
| Mandelate | Pattern E | endotherm having an onset at 141.0° C. and |
| | | peak at 152.8° C. |

### Example 25: Simultaneous Thermogravimetric Analysis and Differential Scanning Calorimetry (TGA/DSC)

Thermogravimetric analysis and differential scanning calorimetry were performed on the same sample simultaneously using a Mettler Toledo TGA/DSC³⁺. Protective and purge gas was nitrogen at flowrates of 20-30 mL/min and 50-100 mL/min respectively. The desired amount of sample (5-10 mg) was weighed directly in a hermetic aluminum pan with pin-hole and analyzed according to the parameters below:

| Method | Ramp | |
|---|---|---|
| Sample size | 5-10 | mg |
| Heating rate | 10.0° | C./min |

| | |
|---|---|
| Temperature range | 30 to 200° C. |

### Characterization of Solid State Forms of Compound I

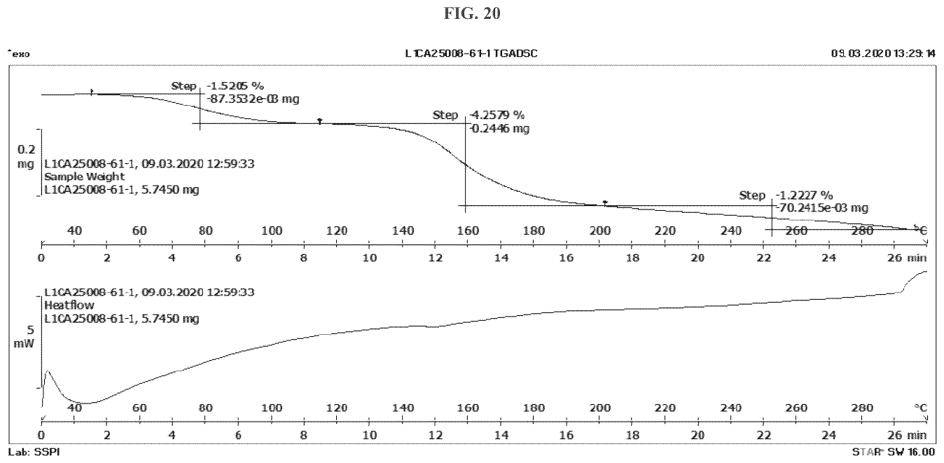

The simultaneous TGA/DSC thermogram for crystalline Pattern B of Compound I Maleate is displayed in FIG. 11. The simultaneous TGA/DSC thermogram for crystalline Pattern C of Compound I Maleate is displayed in FIG. 7. The simultaneous TGA/DSC thermogram for crystalline Pattern D of Compound I Maleate is displayed in FIG. 3. The simultaneous TGA/DSC thermogram for crystalline Pattern E of Compound I Mandelate is displayed in FIG. 15. The simultaneous TGA/DSC thermogram for amorphous Compound I is displayed in FIG. 20.

The Simultaneous TGA/DSC patterns for the solid state forms are as described in the following table:

| Salt Form | Pattern | TGA Pattern | DSC Thermal Events |
|---|---|---|---|
| Free base | amorphous | 1.52% weight | |
| | | loss from 45° C. | |
| | | to 115° C.; | |
| | | 5.48% weight | |
| | | loss from 115° C. | |
| | | to 300° C. | |
| Maleate | Pattern B | >4.7% weight | endotherm with onset |
| | | loss up to 200° C. | at 120.2° C. and peak |
| | | | at 131.4° C. |
| | Pattern C | 0.45% weight | endotherm with onset |
| | | loss up to 170° C. | at 141.7° C. and peak |
| | | | at 152.1° C. |
| | Pattern D | 0.48% weight | endotherm with onset |
| | | loss up to 180° C. | at 158.1° C. and peak |
| | | | at 167.6° C. |
| Mandelate | Pattern E | 1.92% weight | endotherm with onset |
| | | loss up to 170° C. | at 139.8° C. and peak |
| | | | at 154.2° C. |

Results: The TGA pattern of amorphous Compound I is consistent with an amorphous solid with ca. 1.52 wt. % surface water and 5.48 wt. % residual solvent. Crystalline Compound I Maleate Pattern C showed a mass loss of 0.45%, and Pattern D showed a mass loss of 0.48%, both consistent with unsolvated forms. Crystalline Compound I Mandelate Pattern E showed a mass loss of 1.92%, indicating that it retains more residual solvent under the same drying conditions. NMR analysis also showed 1.1 weight % of IPA in mandelate Pattern E.

### Example 26: Dynamic Vapor Sorption (DVS)

DVS was performed using a DVS Intrinsic 1. The sample (20-25 mg) was loaded into a sample pan, suspended from a microbalance and exposed to a humidified stream of nitrogen gas. The sample was held for a minimum of 5 min at each level and only progressed to the next humidity level if there was <0.002% change in weight between measurements (interval: 60 s) or 240 min had elapsed. The following program was used:
1. Equilibration at 50% RH
2. 50% to 2%. (50%, 40%, 30%, 20%, 10%, and 2%)
3. 2% to 95% (2%, 10%, 20%, 30%, 40%, 50%, 60%, 70%, 80%, 90%, and 95%)
4. 95% to 2% (95%, 80%, 70%, 60%, 50%, 40%, 30%, 20%, 10%, and 2%)
5. 2% to 50% (2%, 10%, 20%, 30%, 40%, and 50%)

### Characterization of Solid State Forms of Compound I

The DVS isotherm plot for crystalline Pattern C of Compound I Maleate is displayed in FIG. 8. The DVS isotherm plot for crystalline Pattern D of Compound I Maleate is displayed in FIG. 4. The DVS isotherm plot for crystalline Pattern E of Compound I Mandelate is displayed in FIG. 16.

### Results:

For crystalline Pattern C of Compound I Maleate, DVS analysis indicated a reversible mass gain of 9.18 wt. % from 2 to 95% relative humidity. Slight changes were observed by XRPD following the DVS analysis, indicating some conversion to amorphous material.

For crystalline Pattern D of Compound I Maleate, DVS analysis indicated a reversible mass gain of 1.14 wt. % from 2 to 95% relative humidity. No change was observed by XRPD following the DVS analysis.

For crystalline Pattern E of Compound I Mandelate, DVS analysis indicated a reversible mass gain of 5.68 wt. % from 2 to 95% relative humidity. No change was observed by XRPD following the DVS analysis.

Maleate Pattern D was slightly hygroscopic and stable at high humidity. Maleate Pattern C and mandelate Pattern E were moderately hygroscopic and began to convert to amorphous at high humidity. Both Patterns C and D, as well as mandelate pattern E, were stable for over a week at 40° C. and 75% RH.

### Example 27: Nuclear Magnetic Resonance (NMR) Spectroscopy

Proton NMR was performed on a Bruker Avance 300 MHz spectrometer. Solids are dissolved in 0.75 mL deuterated solvent in a 4 mL vial, transferred to an NMR tube (Wilmad 5 mm thin wall 8″ 200 MHz, 506-PP-8), and analyzed according to the following parameters:

| | |
|---|---|
| Instrument | Bruker Avance 300 MHz spectrometer |
| Temperature | 300 K |
| Probe | 5 mm PABBO BB-1H/DZ-GRD Z104275/0170 |
| Number of Scans | 16-64 |
| Relaxation delay | 1.000 s |
| Pulse width | 14.2500 μs |
| Acquisition time | 2.9999 s |
| Spectrometer frequency | 300.15 Hz |
| Nucleus | ¹H |

### Example 28: High Performance Liquid Chromatography (HPLC)

High performance liquid chromatography (HPLC) was conducted using an Agilent 1220 Infinity LC. Flow rate range was 0.2-5.0 mL/min, operating pressure range was 0-600 bar, temperature range was 5° C. above ambient to 60° C., and wavelength range was 190-600 nm.

The HPLC method used in this study is shown below

| Mobile Phase A | 50 mM Ammonium bicarbonate in distilled water | |
|---|---|---|
| Mobile Phase B | ACN | |
| Diluent | ACN:water (70:30 vol) | |
| Injection Volume | 10 μL | |
| Monitoring | 210 nm | |
| wavelength | | |
| Column | Waters C-18, 4.6 × 150 mm, 3.5 μm | |
| Column temperature | 40° C. | |
| Flow rate | 0.8 mL/min | |
| Gradient Method | Time (min) | % A |
| | 0 | 32 |
| | 2 | 32 |
| | 30 | 65 |
| | 40 | 85 |
| | 45 | 85 |
| | 46 | 32 |
| | 52 | 32 |

### Example 29: Single Crystal X-Ray Diffraction (SCXRD) of Crystalline Pattern D of Compound I Maleate

### Sample Preparation

Crystals of suitable quality for single crystal structure determination of Compound I maleate were obtained from a vapor diffusion experiment using IPA as solvent and MIBK as antisolvent at 60° C. using 32.2 mg of Compound I maleate dissolved in 0.25 mL of IPA. Approximately 3 mL of MIBK was used as antisolvent.

### Data Collection and Data Reduction

Diffraction data (φ- and ω-scans) were collected at 100K on a Bruker-AXS X8 Kappa diffractometer coupled to a Bruker Photon2 CPAD detector using Cu Kα radiation (λ=1.54178 Å) from an IμS microsource. Data reduction was carried out with the program SAINT and semi-empirical absorption correction based on equivalents was performed with the program SADABS.

### Structure Solution and Refinement

The structure was solved with dual-space methods using the program SHELXD and refined against F² on all data with SHELXL using established refinement techniques. All non-hydrogen atoms were refined anisotropically. All carbon-bound hydrogen atoms were placed in geometrically calculated positions and refined using a riding model while constraining their Uᵢₛₒ to 1.2 times the U_{eq} of the atoms to which they bind (1.5 times for CH₃ groups). Coordinates for the hydrogen atoms connected to nitrogen and oxygen were taken from the difference Fourier synthesis and those hydrogen atoms were subsequently refined semi-freely with the help of distance restraints on the O—H and N—H distances (target values 0.84(2) Å for OH, 0.88(2) Å for amid NH, and 0.91(2) Å for amine NH).

### Crystal Structure

Compound I maleate was found to crystallize in the monoclinic chiral space group C2 with two molecules of Compound I and two maleate ions in the asymmetric unit. The compound crystallized in the monoclinic chiral space group C2, with final R indices of R1=0.0661 (I>2σ(I)) and wR2=0.1794 (all reflections).

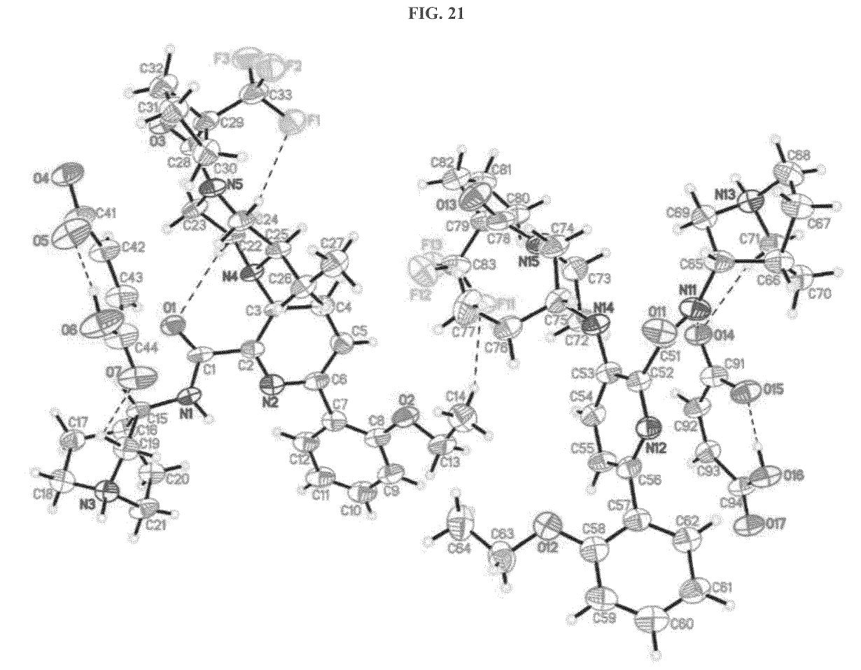

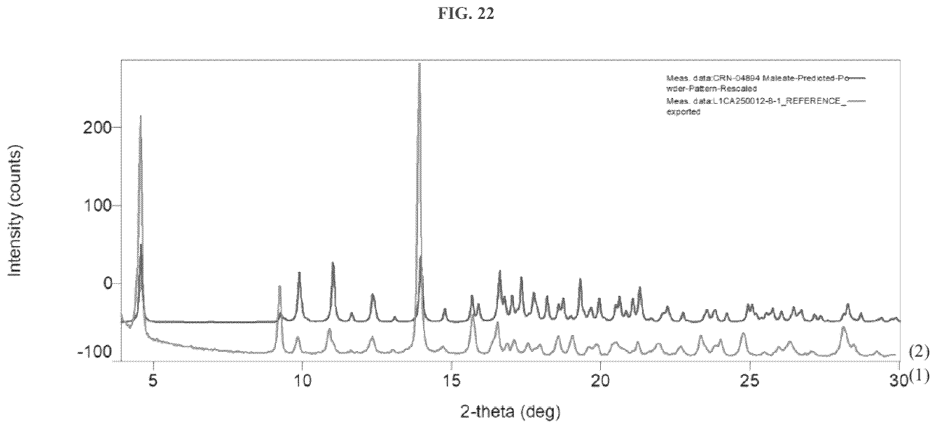

Thermal ellipsoid representation at the 50% probability level for all atoms in the asymmetric unit of the structure of Compound I maleate is shown in FIG. 21, and the corresponding crystal data can be seen in Table 15. The predicted XRPD pattern (2) and the actual maleate XRPD pattern (1) are shown in FIG. 22, and are in good agreement (one minor extra peak seen at 15.88° 2θ).

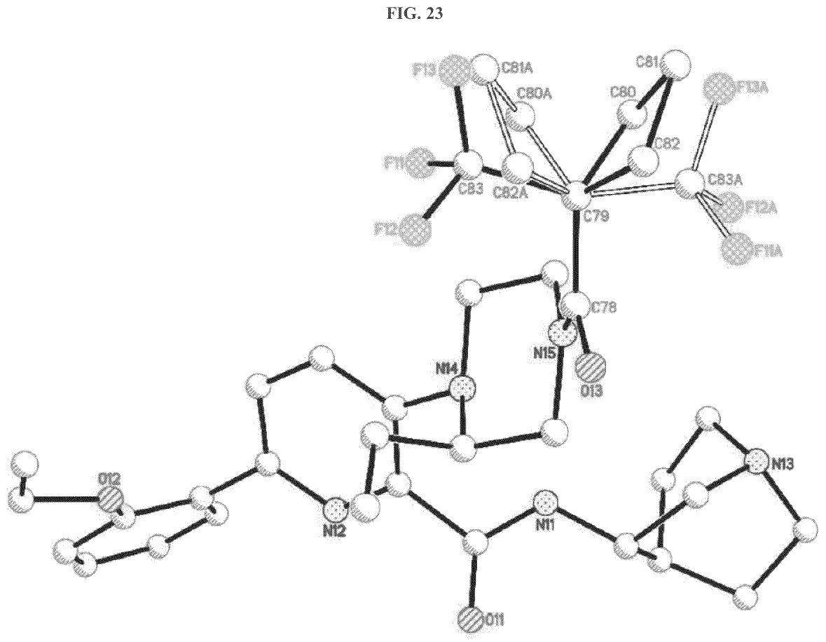

During the structure refinement, it was identified that, trifluoromethyl-cyclobutyl group in one of the crystallographically independent molecules was refined as disordered over two positions. The disorder corresponds to an approximate 180° rotation about the C78-C79 bond (FIG. 23). The disorder ratio was refined freely and converged at 0.860(7).

The molecule at hand is chiral and the absolute structure could be determined based on resonant scattering data: The Flack-x parameters as calculated by the Parsons method refined to −0.06(14). Analysis of the anomalous signal using the method introduced by Hooft & Spek calculates the probability of the absolute structure to be correct to 1, the probability of the structure to be a racemic twin to 0.2×10⁻³, and the probability of the absolute structure to be incorrect to 0. This method also gives rise to an absolute structure parameter, the Hooft-y, which is directly comparable to the Flack-x. The Hooft-y was calculated to 0.00(12). Therefore, it can be determined with reasonably high confidence that the chiral atoms of the Compound I molecule have the configuration C15: S and C25: R in one independent molecule and C65: S/C65: R in the other (both molecules have the same configuration).

The crystal structure of Crystalline Pattern D of Compound I Maleate was determined at 100 K and a summary of the structural data and refinement can be found in the following tables.

TABLE 15
Crystal Data and Structure Refinement -
Compound I Maleate, Pattern D

| | |
|---|---|
| Temperature | 100(2) K |
| Wavelength | 1.54178 Å |
| Crystal System | monoclinic |
| Space Group | C2 |
| a | 18.7203(7) Å |
| b | 10.2473(3) Å |
| c | 38.0119(14) Å |
| α | 90° |
| β | 97.109(3)° |
| γ | 90° |
| V | 7235.9(4) Å³ |
| Z | 8 |
| Calculated Density | 1.340 Mg/m3 |
| Absorption coefficient | 0.867 mm⁻¹ |
| F(000) | 3088 |
| Crystal size | 0.095 × 0.040 × 0.005 mm³ |
| Theta range for data collection | 2.343 to 54.237° |
| Index Ranges | −18 <= h <= 19 |
| | −10 <= k <= 10 |
| | −40 <= l <= 40 |
| Reflections collected | 66337 |
| Independent reflections | 8813 [Rᵢₙₜ = 0.1717] |
| Completeness to theta = 54.237° | 100.0% |
| Absorption correction | Semi-empirical from equivalents |
| Refinement method | Full-matrix least-squares on F² |
| Data/restraints/parameters | 8813/1987/1023 |
| Goodness-of-fit on F² | 1.054 |
| Final R indices [I > 2σ(I)] | R1 = 0.0661, wR2 = 0.1536 |
| R indices (all data) | R1 = 0.1054, wR2 = 0.1794 |
| Absolute structure parameter | −0.06(14) |
| Largest diff. peak and hole | 0.240 and −0.310 e · Å⁻³ |

TABLE 16
Atomic coordinates (×10⁴) and equivalent isotropic
displacement parameters (Å² × 10³) for
Compound I Maleate, Pattern D at 100 K

| Atom | x | Y | z | U(eq)* |
|---|---|---|---|---|
| O(1) | 7958(3) | 7054(6) | 681(2) | 42(2) |
| O(2) | 7969(4) | 11189(7) | 2243(2) | 53(2) |
| O(3) | 8995(4) | 1594(6) | 1041(2) | 45(2) |
| N(1) | 8517(4) | 9022(7) | 708(2) | 34(2) |
| N(2) | 7758(4) | 9392(8) | 1293(2) | 40(2) |
| N(3) | 8879(4) | 10963(8) | −19(2) | 37(2) |
| N(4) | 8573(4) | 6105(7) | 1389(2) | 34(2) |
| N(5) | 9130(4) | 3597(7) | 1286(2) | 39(2) |
| C(1) | 8169(5) | 8023(9) | 843(3) | 40(2) |
| C(2) | 8047(5) | 8233(9) | 1227(2) | 35(2) |
| C(3) | 8219(5) | 7262(8) | 1483(3) | 34(2) |
| C(4) | 8046(5) | 7512(10) | 1823(3) | 43(2) |
| C(5) | 7750(5) | 8717(10) | 1893(3) | 45(3) |
| C(6) | 7614(5) | 9626(9) | 1625(3) | 39(2) |
| C(7) | 7295(5) | 10930(10) | 1675(3) | 42(2) |
| C(8) | 7461(5) | 11684(10) | 1979(3) | 46(2) |
| C(9) | 7129(6) | 12903(10) | 2007(3) | 54(3) |
| C(10) | 6648(6) | 13353(12) | 1734(3) | 58(3) |
| C(11) | 6485(6) | 12642(10) | 1429(3) | 56(3) |
| C(12) | 6810(5) | 11442(10) | 1399(3) | 46(3) |
| C(13) | 8367(6) | 12136(11) | 2472(3) | 60(3) |
| C(14) | 8978(6) | 11386(13) | 2677(3) | 65(3) |
| C(15) | 8532(4) | 9065(9) | 322(3) | 33(2) |
| C(16) | 7830(5) | 9612(10) | 125(3) | 40(2) |
| C(17) | 7882(5) | 9534(11) | −269(3) | 46(3) |
| C(18) | 8482(6) | 10467(11) | −356(3) | 50(3) |
| C(19) | 9162(5) | 9897(10) | 221(3) | 42(2) |
| C(20) | 7726(5) | 11017(9) | 235(3) | 43(2) |
| C(21) | 8391(5) | 11797(9) | 171(3) | 49(3) |
| C(22) | 8071(5) | 5024(8) | 1287(3) | 35(2) |
| C(23) | 8438(5) | 4014(9) | 1084(3) | 38(2) |
| C(24) | 9583(5) | 4644(9) | 1453(3) | 40(2) |
| C(25) | 9180(5) | 5637(9) | 1645(3) | 38(2) |
| C(26) | 9682(5) | 6743(10) | 1778(3) | 43(3) |
| C(27) | 10300(5) | 6300(11) | 2053(3) | 53(3) |
| C(28) | 9359(5) | 2391(9) | 1231(3) | 38(2) |
| C(29) | 10097(5) | 1918(9) | 1406(3) | 41(2) |
| C(30) | 10789(5) | 2751(10) | 1391(3) | 45(3) |
| C(31) | 11184(5) | 1565(10) | 1265(3) | 49(3) |
| C(32) | 10449(5) | 828(10) | 1193(3) | 50(3) |
| C(33) | 10021(5) | 1471(10) | 1775(3) | 52(3) |
| F(1) | 9804(3) | 2419(6) | 1985(2) | 53(2) |
| F(2) | 10641(3) | 1012(6) | 1952(2) | 61(2) |
| F(3) | 9539(4) | 491(6) | 1779(2) | 66(2) |
| O(4) | 10196(4) | 2443(7) | 386(2) | 51(2) |
| O(5) | 11026(4) | 3976(7) | 385(3) | 65(2) |
| O(6) | 11095(4) | 6344(8) | 442(3) | 76(3) |
| O(7) | 10363(4) | 7973(8) | 524(2) | 67(2) |
| C(41) | 10395(6) | 3594(11) | 415(3) | 46(3) |
| C(42) | 9829(5) | 4574(11) | 475(3) | 47(3) |
| C(43) | 9864(5) | 5852(10) | 515(3) | 50(3) |
| C(44) | 10460(6) | 6810(12) | 492(3) | 56(3) |
| O(11) | 12536(3) | 9690(7) | 4111(2) | 49(2) |
| O(12) | 11734(4) | 13980(7) | 3086(2) | 52(2) |
| O(13) | 9886(4) | 5473(8) | 3146(2) | 66(2) |
| N(11) | 11606(5) | 9538(9) | 4437(2) | 47(2) |
| N(12) | 11831(4) | 12162(8) | 3921(2) | 38(2) |
| N(13) | 11137(4) | 7237(8) | 5025(2) | 43(2) |
| N(14) | 10504(4) | 9499(8) | 3772(2) | 42(2) |
| N(15) | 9821(4) | 7122(8) | 3533(2) | 48(2) |
| C(51) | 11931(5) | 9993(9) | 4165(3) | 38(2) |
| C(52) | 11488(5) | 11019(9) | 3943(3) | 36(2) |
| C(53) | 10783(5) | 10773(9) | 3776(3) | 38(2) |
| C(54) | 10431(5) | 11842(9) | 3606(3) | 39(2) |
| C(55) | 10771(5) | 13021(9) | 3585(3) | 42(3) |
| C(56) | 11482(5) | 13135(9) | 3738(3) | 39(2) |
| C(57) | 11905(5) | 14343(9) | 3696(3) | 42(2) |
| C(58) | 12033(5) | 14752(10) | 3359(3) | 45(2) |
| C(59) | 12455(6) | 15839(10) | 3321(3) | 54(3) |
| C(60) | 12736(6) | 16537(12) | 3611(3) | 56(3) |
| C(61) | 12615(6) | 16174(11) | 3947(3) | 53(3) |
| C(62) | 12197(5) | 15058(10) | 3990(3) | 46(3) |
| C(63) | 11706(7) | 14470(13) | 2728(3) | 61(3) |
| C(64) | 11264(6) | 13488(13) | 2501(3) | 67(4) |
| C(65) | 11913(5) | 8459(9) | 4654(3) | 45(2) |
| C(66) | 12176(5) | 8788(10) | 5040(3) | 44(2) |
| C(67) | 12414(5) | 7525(10) | 5230(3) | 51(3) |
| C(68) | 11742(5) | 6740(11) | 5280(3) | 50(3) |
| C(69) | 11356(6) | 7341(10) | 4659(3) | 50(3) |
| C(70) | 11568(5) | 9416(10) | 5214(3) | 45(3) |
| C(71) | 10912(5) | 8541(10) | 5138(3) | 46(3) |
| C(72) | 9707(5) | 9418(10) | 3699(3) | 44(3) |
| C(73) | 9461(6) | 8050(10) | 3749(3) | 54(3) |
| C(74) | 10609(5) | 7208(10) | 3607(3) | 52(3) |
| C(75) | 10857(5) | 8588(9) | 3538(3) | 44(2) |
| C(76) | 10722(6) | 8990(10) | 3146(3) | 47(3) |
| C(77) | 11126(6) | 8161(13) | 2904(3) | 69(4) |
| C(78) | 9524(5) | 6282(10) | 3291(3) | 50(3) |
| C(79) | 8714(5) | 6229(9) | 3179(3) | 45(2) |
| C(80) | 8125(6) | 6287(12) | 3447(3) | 49(3) |
| C(81) | 7732(7) | 5155(14) | 3245(4) | 55(4) |
| C(82) | 8388(6) | 4895(11) | 3052(4) | 53(3) |
| C(83) | 8540(6) | 7255(11) | 2896(3) | 49(3) |
| F(11) | 8599(3) | 8476(6) | 3015(2) | 53(2) |
| F(12) | 8988(4) | 7161(8) | 2643(2) | 62(2) |
| F(13) | 7875(4) | 7143(10) | 2718(2) | 56(3) |
| C(80A) | 8200(20) | 7380(40) | 3180(16) | 53(7) |
| C(81A) | 7850(50) | 6950(130) | 2810(20) | 54(9) |
| C(82A) | 8490(30) | 6030(60) | 2770(7) | 49(7) |
| C(83A) | 8562(16) | 5000(30) | 3380(8) | 52(6) |
| F(11A) | 8950(17) | 3940(30) | 3309(10) | 47(9) |
| F(12A) | 8761(19) | 5220(30) | 3728(8) | 53(9) |
| F(13A) | 7872(16) | 4640(40) | 3328(13) | 62(11) |
| O(14) | 10087(4) | 10514(7) | 4512(2) | 44(2) |
| O(15) | 10762(4) | 12177(7) | 4717(2) | 49(2) |
| O(16) | 10681(4) | 14547(7) | 4724(2) | 53(2) |
| O(17) | 9854(4) | 16042(7) | 4579(2) | 51(2) |
| C(91) | 10164(5) | 11706(11) | 4551(3) | 42(2) |
| C(92) | 9574(6) | 12581(10) | 4411(3) | 45(3) |
| C(93) | 9525(6) | 13868(10) | 4419(3) | 44(3) |
| C(94) | 10033(6) | 14877(10) | 4582(3) | 44(3) |

*U(eq) is defined as one third of the trace of the orthogonalized Uij tensor.

We claim:

1. A maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I).

2. The maleate salt of claim 1, wherein the maleate salt of Compound I is crystalline.

3. A crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern D and is characterized as having:
an X-ray powder diffraction (XRPD) pattern substantially the same as shown in FIG. 1, as measured using Cu (Kα) radiation; or
an XRPD pattern with reflections at about 4.7±0.2° 2-Theta, 9.4±0.2° 2-Theta, 11.0±0.2° 2-Theta, and 14.0±0.2° 2-Theta, as measured using Cu (Kα) radiation; or
a Differential Scanning Calorimetry (DSC) thermogram substantially the same as shown in FIG. 2; or
a simultaneous Thermogravimetric Analysis (TGA)/DSC thermogram substantially the same as shown in FIG. 3; or
a DSC thermogram with an endotherm having an onset at 161.6° C. and peak at 168.4° C.; or an endotherm with onset at 158.1° C. and peak at 167.6° C.; or
a TGA pattern with a 0.48% weight loss up to 180° C.; or
unit cell parameters substantially equal to the following at 100 K:

| Crystal System | monoclinic |
|---|---|
| Space Group | C2 |
| a | 18.7203(7) Å |
| b | 10.2473(3) Å |
| c | 38.0119(14) Å |
| α | 90° |
| β | 97.109(3)° |
| γ | 90° |
| V | 7235.9(4) Å³ |
| Z | 8 |
| Calculated Density | 1.340 Mg/m3 |
| Independent reflections | 8813; |

or
a Dynamic Vapour Sorption (DVS) isotherm plot substantially the same as shown in FIG. 4; or
a reversible mass gain of 1.14 wt. % from 2 to 95% relative humidity (RH), an unchanged XRPD after DVS analysis from 2 to 95% RH, an unchanged XRPD after storage for at least one week at 40° C. and 75% RH, or a combination thereof;
or a combination thereof.

4. The crystalline form of the maleate salt of Compound I of claim 3, wherein the crystalline form is characterized as having an XRPD pattern substantially the same as shown in FIG. 1, as measured using Cu (Kα) radiation.

5. The crystalline form of the maleate salt of Compound I of claim 3, wherein the crystalline form is characterized as having an XRPD pattern with reflections at about 4.7±0.2° 2-Theta, 9.4±0.2° 2-Theta, 11.0±0.2° 2-Theta, and 14.0±0.2° 2-Theta, as measured using Cu (Kα) radiation.

6. The crystalline form of the maleate salt of Compound I of any one of claims 3-5, wherein the crystalline form is characterized as having:
a DSC thermogram substantially the same as shown in FIG. 2; or
a DSC thermogram with an endotherm having an onset at 161.6° C. and peak at 168.4° C.

7. The crystalline form of the maleate salt of Compound I of any one of claims 3-5, wherein the crystalline form is characterized as having:
a simultaneous TGA/DSC thermogram substantially the same as shown in FIG. 3; or
a DSC thermogram with an endotherm with onset at 158.1° C. and peak at 167.6° C.; or
a TGA with a 0.48% weight loss up to 180° C.

8. The crystalline form of the maleate salt of Compound I of claim 3, wherein the crystalline form is characterized as having unit cell parameters substantially equal to the following at 100 K:

| Crystal System | monoclinic |
|---|---|
| Space Group | C2 |
| a | 18.7203(7) Å |
| b | 10.2473(3) Å |
| c | 38.0119(14) Å |
| α | 90° |
| β | 97.109(3)° |
| γ | 90° |
| V | 7235.9(4) Å³ |
| Z | 8 |
| Calculated Density | 1.340 Mg/m3 |
| Independent reflections | 8813. |

9. The crystalline form of the maleate salt of Compound I of claim 3, wherein the crystalline form is characterized as having:
an X-ray powder diffraction (XRPD) pattern substantially the same as shown in FIG. 1, as measured using Cu (Kα) radiation; and
a Differential Scanning Calorimetry (DSC) thermogram substantially the same as shown in FIG. 2; or
a simultaneous Thermogravimetric Analysis (TGA)/DSC thermogram substantially the same as shown in FIG. 3.

10. The crystalline form of the maleate salt of Compound I of claim 3, wherein the crystalline form is characterized as having:
an XRPD pattern with reflections at about 4.7±0.2° 2-Theta, 9.4±0.2° 2-Theta, 11.0±0.2° 2-Theta, and 14.0±0.2° 2-Theta, as measured using Cu (Kα) radiation; and
a DSC thermogram with an endotherm having an onset at 161.6° C. and peak at 168.4° C.; or an endotherm with onset at 158.1° C. and peak at 167.6° C.; or
a TGA pattern with a 0.48% weight loss up to 180° C.

11. The crystalline form of the maleate salt of Compound I of claim 3, wherein the crystalline form is anhydrous.

12. A crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern C and is characterized as having:
an X-ray powder diffraction (XRPD) pattern substantially the same as shown in FIG. 5, as measured using Cu (Kα) radiation; or
an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; or
a Differential Scanning Calorimetry (DSC) thermogram substantially the same as shown in FIG. 6; or
a simultaneous Thermogravimetric Analysis (TGA)/DSC thermogram substantially the same as shown in FIG. 7; or
a DSC thermogram with an endotherm having an onset at 144.1° C. and peak at 150.7° C.; or an endotherm with onset at 141.7° C. and peak at 152.1° C.; or
a TGA pattern with a 0.45% weight loss up to 170° C.; or
a Dynamic Vapour Sorption (DVS) isotherm plot substantially the same as shown in FIG. 8; or
a reversible mass gain of 9.18 wt. % from 2 to 95% relative humidity (RH), an XRPD with slight changes showing some conversion to the amorphous maleate salt of Compound I after DVS analysis from 2 to 95% RH, an unchanged XRPD after storage for at least one week at 40° C. and 75% RH, or a combination thereof;
or a combination thereof.

13. The crystalline form of the maleate salt of Compound I of claim 12, wherein the crystalline form is characterized as having:
an X-ray powder diffraction (XRPD) pattern substantially the same as shown in FIG. 5, as measured using Cu (Kα) radiation; and
a Differential Scanning Calorimetry (DSC) thermogram substantially the same as shown in FIG. 6; or
a simultaneous Thermogravimetric Analysis (TGA)/DSC thermogram substantially the same as shown in FIG. 7; or
a Dynamic Vapour Sorption (DVS) isotherm plot substantially the same as shown in FIG. 8.

14. The crystalline form of the maleate salt of Compound I of claim 12, wherein the crystalline form is characterized as having:
an XRPD pattern with reflections at about 4.5±0.2° 2-Theta, 9.0±0.2° 2-Theta, 13.5±0.2° 2-Theta, and 18.4±0.2° 2-Theta, as measured using Cu (Kα) radiation; or
a Differential Scanning Calorimetry (DSC) thermogram substantially the same as shown in FIG. 6; and
a DSC thermogram with an endotherm having an onset at 144.1° C. and peak at 150.7° C.; or an endotherm with onset at 141.7° C. and peak at 152.1° C.; or
a TGA pattern with a 0.45% weight loss up to 170° C.

15. A crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern B and is characterized as having:
an X-ray powder diffraction (XRPD) pattern substantially the same as shown in FIG. 9, as measured using Cu (Kα) radiation; or
an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; or
a Differential Scanning Calorimetry (DSC) thermogram substantially the same as shown in FIG. 10; or
a simultaneous Thermogravimetric Analysis (TGA)/DSC thermogram substantially the same as shown in FIG. 11; or
a DSC thermogram with an endotherm having an onset at 141.4° C. and peak at 150.5° C.; or an endotherm with onset at 120.2° C. and peak at 131.4° C.; or
a TGA pattern with a >4.7% weight loss up to 200° C.; or
an XRPD that converts to Pattern C after sitting at ambient conditions for one week;
or a combination thereof.

16. The crystalline form of the maleate salt of Compound I of claim 15, wherein the crystalline maleate salt of Compound I is characterized as having:
an X-ray powder diffraction (XRPD) pattern substantially the same as shown in FIG. 9, as measured using Cu (Kα) radiation; and
a Differential Scanning Calorimetry (DSC) thermogram substantially the same as shown in FIG. 10; or
a simultaneous Thermogravimetric Analysis (TGA)/DSC thermogram substantially the same as shown in FIG. 11.

17. The crystalline form of the maleate salt of Compound I of claim 15, wherein the crystalline maleate salt of Compound I is characterized as having:
an XRPD pattern with reflections at about 4.1±0.2° 2-Theta, 8.2±0.2° 2-Theta, 12.3±0.2° 2-Theta, and 16.4±0.2° 2-Theta, as measured using Cu (Kα) radiation; and
a DSC thermogram with an endotherm having an onset at 141.4° C. and peak at 150.5° C.; or an endotherm with onset at 120.2° C. and peak at 131.4° C.; or
a TGA pattern with a >4.7% weight loss up to 200° C.

18. A crystalline form of the maleate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline maleate salt of Compound I is crystalline Pattern A and is characterized as having:
an X-ray powder diffraction pattern (XRPD) substantially the same as shown in FIG. 12, as measured using Cu (Kα) radiation; or
an XRPD pattern with reflections at about 4.2±0.2° 2-Theta, 8.3±0.2° 2-Theta, and 12.5±0.2° 2-Theta, as measured using Cu (Kα) radiation; or
an XRPD that converts to Pattern C after sitting at ambient conditions for about three days; or
an XRPD that converts to Pattern C after drying in a vacuum oven at 50° C., 10⁻²-10⁻¹ Torr for 20 h;
or a combination thereof.

19. The crystalline form of the maleate salt of Compound I of claim 18, wherein the crystalline maleate salt of Compound I is characterized as having:
an X-ray powder diffraction pattern (XRPD) substantially the same as shown in FIG. 12, as measured using Cu (Kα) radiation.

20. The crystalline form of the maleate salt of Compound I of claim 18, wherein the crystalline maleate salt of Compound I is characterized as having:
an XRPD pattern with reflections at about 4.2±0.2° 2-Theta, 8.3±0.2° 2-Theta, and 12.5±0.2° 2-Theta, as measured using Cu (Kα) radiation.

21. The crystalline form of the maleate salt of Compound I of any one of claims 3-11, wherein the crystalline maleate salt of Compound I is crystalline Pattern D, and optionally further comprises: the crystalline Pattern C of any one of claims 12-14, the crystalline Pattern B of any one of claims 15-17, the crystalline Pattern A of any one of claims 18-20, or amorphous maleate salt of Compound I, or a combination thereof.

22. A mandelate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the mandelate salt of Compound I is crystalline.

23. A crystalline form of the mandelate salt of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I), wherein the crystalline mandelate salt of Compound I is crystalline Pattern E and is characterized as having:
an X-ray powder diffraction pattern (XRPD) substantially the same as shown in FIG. 13, as measured using Cu (Kα) radiation; or
an XRPD with X-ray diffraction pattern reflections at about 5.6±0.2° 2-Theta, 10.5±0.2° 2-Theta, 14.9±0.2° 2-Theta, and 16.5±0.2° 2-Theta, as measured using Cu (Kα) radiation; or
a Differential Scanning Calorimetry (DSC) thermogram substantially the same as shown in FIG. 14; or
a simultaneous Thermogravimetric Analysis (TGA)/DSC thermogram substantially the same as shown in FIG. 15; or
a DSC thermogram with an endotherm having an onset at 141.0° C. and peak at 152.8° C.; or an endotherm with onset at 139.8° C. and peak at 154.2° C.; or
a TGA pattern with a 1.92% weight loss up to 170° C.; or
a Dynamic Vapour Sorption (DVS) isotherm plot substantially the same as shown in FIG. 16; or
a reversible mass gain of 5.68 wt. % from 2 to 95% relative humidity (RH), an unchanged XRPD after DVS analysis from 2 to 95% RH, an unchanged XRPD after storage for at least one week at 40° C. and 75% RH, or a combination thereof;
or a combination thereof.

24. The crystalline form of the mandelate salt of Compound I of claim 23, wherein the crystalline mandelate salt of Compound I is characterized as having:
an X-ray powder diffraction pattern (XRPD) substantially the same as shown in FIG. 13, as measured using Cu (Kα) radiation; and
a Differential Scanning Calorimetry (DSC) thermogram substantially the same as shown in FIG. 14; or
a simultaneous Thermogravimetric Analysis (TGA)/DSC thermogram substantially the same as shown in FIG. 15; or
a Dynamic Vapour Sorption (DVS) isotherm plot substantially the same as shown in FIG. 16.

25. The crystalline form of the mandelate salt of Compound I of claim 23, wherein the crystalline mandelate salt of Compound I is characterized as having:
an XRPD with X-ray diffraction pattern reflections at about 5.6±0.2° 2-Theta, 10.5±0.2° 2-Theta, 14.9±0.2° 2-Theta, and 16.5±0.2° 2-Theta, as measured using Cu (Kα) radiation; and
a DSC thermogram with an endotherm having an onset at 141.0° C. and peak at 152.8° C.; or an endotherm with onset at 139.8° C. and peak at 154.2° C.; or
a TGA pattern with a 1.92% weight loss up to 170° C.

26. A pharmaceutical composition comprising the maleate salt of any one of claims 1 to 21; and at least one pharmaceutically acceptable excipient.

27. A pharmaceutical composition comprising the mandelate salt of any one of claims 22 to 25; and at least one pharmaceutically acceptable excipient.

28. The pharmaceutical composition of claim 26 or 27, wherein the pharmaceutical composition is in the form of a solid form pharmaceutical composition.

29. The pharmaceutical composition of claim 28, wherein the pharmaceutical composition is in the form of a tablet, a pill, or a capsule.

30. A process for the preparation of Crystalline Pattern D of N-[(3S)-1-azabicyclo[2.2.2]octan-3-yl]-6-(2-ethoxyphenyl)-3-[(2R)-2-ethyl-4-[1-(trifluoromethyl)-cyclobutanecarbonyl]piperazin-1-yl]pyridine-2-carboxamide (Compound I) maleate: 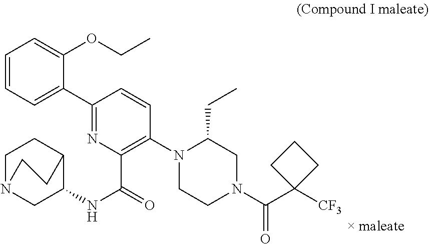 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]
comprising:
(6) contacting Compound I with maleic acid in a suitable solvent to form a mixture;
(7) adding a suitable antisolvent to the mixture and seeding the mixture with crystals of Pattern D of Compound I maleate;
(8) heating the mixture at a suitable temperature for a sufficient amount of time to obtain a slurry;
(9) cooling the slurry at a suitable cooling rate; and
(10) filtering the slurry to obtain Crystalline Pattern D of Compound I maleate.

31. The process of claim 30, wherein the suitable solvent in step (1) is ethanol, isopropanol, acetone, acetonitrile, methyl acetate, ethyl acetate, methyl isobutyl ketone (MIBK), water, or a combination thereof.

32. The process of claim 30, wherein the suitable solvent of step (1) is a mixture of isopropanol and MIBK.

33. The process of any one of claims 30 to 32, wherein the mixture of step (1) is heated to about 50° C.

34. The process of any one of claims 30 to 33, wherein the mixture of step (1) comprises about 1.1 equivalents of maleic acid and about 6 volumes of a 5:1 mixture of isopropanol and MIBK, relative to the amount of Compound I in the mixture.

35. The process of any one of claims 30 to 33, wherein the antisolvent in step (2) is methyl tert-butyl ether (MtBE), MIBK, water, heptane, or a combination thereof.

36. The process of any one of claims 30 to 33, wherein the antisolvent in step (2) is heptane.

37. The process of claim 35, wherein from about 4 volumes to about 10 volumes of heptane relative to the amount of Compound I is added to the mixture in step (2).

38. The process of any one of claims 30 to 37, wherein the amount of seed crystals of Pattern D added to the mixture in step (2) is about 0.05%, about 0.10%, about 0.15%, about 0.20%, about 0.25%, about 0.30%, about 0.35%, about 0.40%, about 0.45%, about 0.5%, about 0.60%, about 0.70%, about 0.80%, about 0.90%, or about 0.10%, relative to the amount of Compound I in the mixture.

39. The process of any one of claims 30 to 38, wherein the mixture is heated to a temperature of from about 40° C. to about 50° C. in step (3).

40. The process of any one of claims 30 to 39, wherein the mixture is heated to a temperature of about 50° C. for at least 8 h, at least 12 h, at least 18 h, or more in step (3).

41. The process of any one of claims 30 to 40, wherein the mixture is heated to a temperature of about 50° C. for about 18 h in step (3).

42. The process of any one of claims 30 to 41, wherein the slurry is cooled to a temperature of about 20° C. at a rate of at most 2.5° C./min in step (4).

43. The process of any one of claims 30 to 41, wherein the slurry is cooled to a temperature of about 20° C. over about 15 min, about 30 min, about 45 min, about 60 min or more in step (4).

44. The process of any one of claims 30 to 41, wherein the slurry is cooled to a temperature of about 20° C. over about 45 min in step (4).

45. The process of any one of claims 30 to 44, wherein the Crystalline Pattern D of Compound I maleate obtained in step (5) after filtration is dried under vacuum.

46. The process of any one of claims 30 to 45, further comprising recrystallizing the Crystalline Pattern D of Compound I maleate obtained in step (5).

47. The process of claim 46, wherein recrystallizing the Crystalline Pattern D of Compound I maleate comprises:
viii. contacting Compound I Maleate Pattern D with a suitable solvent to obtain a mixture;
ix. heating the mixture of step (i) to obtain a solution;
x. seeding the solution with crystals of Pattern D of Compound I maleate to obtain a mixture;
xi. adding a suitable antisolvent to the mixture over a suitable amount of time;
xii. heating the mixture for a suitable amount of time to obtain a slurry;
xiii. cooling the slurry at a suitable cooling rate; and
xiv. filtering the slurry to obtain Crystalline Pattern D of Compound I maleate.

48. The process of claim 47, wherein the suitable solvent in step (i) is ethanol, isopropanol, acetone, methyl acetate, or a combination thereof; and wherein from about 4 volumes to about 10 volumes of solvent is used in step (i), relative to the amount of Compound I in the mixture.

49. The process of claim 47, wherein about 4 volumes of isopropanol is used in step (i), relative to the amount of Compound I in the mixture.

50. The process of any one of claims 47 to 49, wherein the mixture is heated to a temperature of from about 30° C. to about 50° C. in step (ii).

51. The process of any one of claims 47 to 50, further comprising cooling the solution obtained in step (ii) to a temperature of about 30° C. over about 2 hours prior to the seeding of step (iii).

52. The process of any one of claims 47 to 51, wherein the amount of seed crystals of Pattern D added to the mixture in step (iii) is about 0.25%, about 0.50%, about 0.75%, about 1.0%, about 1.25%, about 1.50%, about 1.75%, about 2.0%, about 2.5%, about 3.0%, about 3.5%, about 4.0%, about 4.5%, or about 5.0%, relative to the amount of Compound I in the mixture.

53. The process of any one of claims 47 to 52, wherein the suitable antisolvent of step (iv) is MtBE, heptane, or a combination thereof; and wherein from about 3 volumes to about 10 volumes of solvent is used in step (iv), relative to the amount of Compound I in the mixture.

54. The process of any one of claims 47 to 53, wherein the suitable antisolvent of step (iv) is MtBE; and wherein the MtBE is added over at least 1 h, at least 2 h, at least 4 h, at least 6 h, or more.

55. The process of any one of claims 47 to 54, wherein in step (v) the mixture is heated to about 40° C. for about 1 h, about 2 h, or about 3 h.

56. The process of any one of claims 47 to 55, wherein the slurry obtained in step (v) is cooled to a temperature of about 20° C. at a rate of at most 2.5° C./min in step (vi).

57. The process of any one of claims 47 to 55, wherein the slurry obtained in step (v) is cooled to a temperature of about 20° C. over about 30 min, about 60 min, about 90 min, about 120 min, or more in step (vi).

58. The process of any one of claims 47 to 57, the slurry obtained in step (v) is cooled to a temperature of about 20° C. over about 120 min in step (vi).

59. The process of any one of claims 56 to 58, wherein the cooled slurry of step (vi) is maintained at about 20° C. for at least 2 h, at least, 3 h, at least 4 h, or more prior to step (vii).

60. The process of any one of claims 56 to 59, wherein the cooled slurry of step (vi) is maintained at about 20° C. for about 4 h prior to step (vii).

61. The process of any one of claims 47 to 60, wherein the Crystalline Pattern D of Compound I maleate obtained in step (vii) after filtration is dried under vacuum at a temperature of about 50° C.
